(19) United States
(12) Patent Application Publication
(10) Pub. No.: US 2026/0125662 A1
(43) Pub. Date: May 7, 2026

(54) METHOD FOR IMPROVING EFFICIENCY AND ACCURACY OF GENE KNOCK-IN USING NON-RESIDENCE END OF CPF1

(71) Applicant: Yimuhe Hangzhou Biotechnology Co., Ltd., Hangzhou (CN)

(72) Inventors: Anyong XIE, Hangzhou (CN); Ruodan CHEN, Hangzhou (CN); Yi YANG, Hangzhou (CN)

(73) Assignee: Yimuhe Hangzhou Biotechnology Co., Ltd., Hangzhou (CN)

(21) Appl. No.: 19/428,360

(22) Filed: Dec. 22, 2025

Related U.S. Application Data
(63) Continuation of application No. PCT/CN2024/098243, filed on Jun. 7, 2024.

(30) Foreign Application Priority Data
Jun. 21, 2023 (CN) 202310739608.5

(51) Int. Cl. C12N 9/22; C12N 15/11; C12N 15/90

(52) U.S. Cl. CPC C12N 9/226; C12N 15/11; C12N 15/907; C12N 2310/20

(57) ABSTRACT

A method for improving the efficiency and accuracy of gene knock-in using the non-residence end of Cpf1, where the free end of a target DNA and the free end of a donor DNA are generated using single or paired Cpf1, and combined with a complementary 5′-sticky end generated by Cpf1, so as to realize a more efficient and more accurate gene knock-in based on c-NHEJ. The method can perform NHEJ repair more efficiently and more accurately, and the accuracy can be increased from 0% to 100%. The free end of Cpf1 binds to and is protected by NHEJ core factor KU70/KU80, thereby making the ligation via NHEJ more efficient and accurate, which provides a foundation for the use of the free end of Cpf1 to improve the efficiency and accuracy of gene knock-in based on NHEJ, realizes gene knock-in or gene correction, and is suitable for non-dividing cells.

### CROSS-REFERENCE OF THE RELATED APPLICATIONS

This application is a continuation application of International Application No. PCT/CN2024/098243, filed on Jun. 7, 2024, which is based upon and claims priority to Chinese Patent Application No. 202310739608.5, filed on Jun. 21, 2023, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBZD060_SequenceListing.xml, created on Dec. 22, 2025, and is 275,638 bytes in size.

### TECHNICAL FIELD

The present invention relates to a method for improving the efficiency and accuracy of gene knock-in using the Cpf1 non-retained end in the field of biotechnology. Specifically, the content relates to the characteristic that Cpf1 asymmetrically retains at the two ends of a cleavage target after cleaving DNA at a specific site. By using single or paired Cpf1, free endogenous genomic target DNA ends and free donor DNA ends are generated. Combined with the complementary 5′-cohesive ends induced by Cpf1, more efficient and more accurate gene knock-in is achieved through the directional non-homologous end joining (NHEJ) between the free ends of the endogenous genomic target DNA and the free ends of the donor DNA. In non-dividing cells, due to the low activity of homologous recombination (HR), it is difficult to mediate gene knock-in via the HR method. Thus, the present invention provides a novel strategy for accurate and efficient gene knock-in in non-dividing cells.

### BACKGROUND

CRISPR (Clustered Regularly Interspaced Short Palindromic Repeats) gene-editing technology originates from an immune defense mechanism in bacteria and archaea. It consists of two components: Cas nuclease and single-guide RNA (sgRNA). After Cas nuclease assembles with sgRNA to form a stable complex, it begins searching for the target sequence in the genome. Once the protospacer-adjacent motif (PAM) is identified, Cas nuclease interacts with the PAM and denatures the double strand of DNA, allowing the spacer sequence on the sgRNA to complementary pair with single-stranded target DNA and form RNA-DNA hybrids. Subsequently, the activated Cas nuclease cleaves the DNA strand in the RNA-DNA hybrid and the single-stranded non-target DNA strand respectively, generating a DNA double strand break (DSB) with two DNA ends. This DSB is mainly repaired through two evolutionarily conserved endogenous cellular repair pathways: homologous recombination (HR) and non-homologous end joining (NHEJ). HR is primarily active during the S and G2 phases of the cell cycle, and mainly uses sister chromatids as homologous templates to repair DNA replication-coupled DSB. NHEJ operates throughout the entire cell cycle and repairs DNA by ligating the two ends of the DNA break. NHEJ is mainly executed by several core factors, including KU70/KU80, DNA-PKcs, and XRCC4/DNA ligase 4, etc. The NHEJ pathway is commonly referred to as classical NHEJ (c-NHEJ). However, when certain core NHEJ factors fail to participate promptly, NHEJ efficiency decreases, the frequency and length of deletions or insertions increase, and the ligation process further requires the assistance of microhomology (MH) sequences. By manipulating these two endogenous DSB repair pathways, the DSB repair will yield a certain proportion of desired gene-editing products, thereby achieving the goal of gene editing.

Targeted gene knock-in based on DSB repair is a gene-editing strategy that relies on high-accuracy repair pathways. It can be used for accurate insertion of target DNA fragments or genes to achieve: 1) correction of point mutations or other mutation types in target genes; 2) insertion of target genes or reporter systems requiring specific expression at target genomic loci, including SafeHarbor loci; 3) N-terminal/C-terminal tagging or N-terminal/C-terminal gene fusion of target genes. A major strategy for gene knock-in uses the accurate repair mechanism of HR, which requires mediation by homologous sequences or homology arms. This strategy has been widely used in correcting defective genes, accurate insertion of target genes (including fluorescent protein genes or drug selection marker genes), and has shown application potential in generating model animals, producing therapeutic cells (e.g., CAR-T cells), and other related fields of in vivo gene therapy. However, since DSB repair in cells prefers the NHEJ pathway and the HR only occurs in the S and G2 phases of the cell cycle, HR-based gene-editing strategies not only have low efficiency that is difficult to meet requirements for gene editing, but also are not suitable for non-dividing cells lacking S and G2 phases, such as neurons and muscle cells. Therefore, NHEJ has also been developed for gene knock-in, enabling accurate CRISPR/Cas9-induced site-specific integration in various dividing and non-dividing mammalian cells (e.g., dividing cells U2OS, HEK293, HAP1, and non-dividing neurons) as well as in zebrafish. Nevertheless, despite the high efficiency of NHEJ-mediated gene knock-in, the NHEJ junction between the DNA fragment and the genome usually contains various mutations, resulting in low efficiency of accurate gene knock-in. Additionally, since Cas9 mainly generates blunt ends when cleaving DNA, the use of blunt ends for NHEJ-mediated gene knock-in has a 50% probability of target gene fragment inversion. Therefore, improving the efficiency of NHEJ-mediated accurate gene knock-in is an urgent problem to be solved.

Unlike the blunt ends generated by Cas9, the DSB generated by the CRISPR nuclease Cpf1 (also known as Cas12a) upon cleaving the target DNA possesses a 5′ cohesive end with a 5-nt overhang. Obviously, the 5′-cohesive ends complementary to the 5-nt overhangs can not only fix the direction of NHEJ-mediated gene knock-in to avoid the inverted knock-in of the transgene fragment but also potentially improve the efficiency and accuracy of NHEJ-based gene knock-in. The PAM recognized by Cpf1 is mainly 5′-TTTN-3′ (where N is any of the four nucleotides), and may also include PAM variants such as 5′-TTN-3′. The recognized target sequence (i.e., the spacer sequence paired with target sequence) is 21-23 nt. Compared to Cas9, Cpf1 is smaller (containing 1273 amino acids) and its associated sgRNA is shorter (43 nt in full length), making it more convenient to use. Furthermore, Cpf1 has a low off-target effect. Therefore, Cpf1 has unique value in specific gene-editing applications, including NHEJ-mediated gene knock-in. However, even with the complementary 5′ overhangs generated by Cpf1, the efficiency and accuracy of end joining for knock-in gene integration are unstable, fluctuating between high and low levels and varying with different targets. Except for a certain positive correlation with the cleavage efficiency of Cpf1, no other regular patterns have been identified. In fact, despite relevant attempts, there is currently no technical method to stably achieve efficient and accurate NHEJ-based targeted gene knock-in by using the 5′ cohesive end of a 5-nt overhang induced by Cpf1.

After recognizing and binding to the target PAM, the Cpf1-sgRNA unwinds the target DNA from the PAM-proximal end, generating a non-target single strand DNA and a hybrid strand formed by the target single strand and the sgRNA spacer sequence. Subsequent cleavage occurs between 18 nt-19 nt away from the PAM on the non-target single strand DNA and between 23 nt-24 nt away from the PAM on the target strand on the hybrid strand, producing a DNA DSB composed of two 5′-cohesive ends with 5-nt overhangs: one PAM-proximal end and the other PAM-distal end. Given that Cpf1 remains bound to the PAM-proximal end (thus termed the retained end) while releasing the PAM-distal end (thus termed the free end) after DNA cleavage, the conventional view holds that the retained end is protected by Cpf1 from nuclease attack. Consequently, compared to the free end, it exhibits fewer base insertions and deletions and undergoes more accurate repair. However, in investigating how cells repair DSBs with 5′-cohesive ends induced by Cpf1 cleavage, we for the first time discovered that the end retention asymmetry of Cpf1 influences the efficiency and accuracy of NHEJ repair. In fact, we found that the Cpf1-free end is more susceptible to binding and protection by KU70/KU80, thereby utilizing c-NHEJ for repair with higher efficiency and accuracy. In contrast, the Cpf1-retained end hinders the binding of KU70/KU80 and thus lacks such protection, which leads to more likely to have base insertions and deletions. This finding lays the foundation for establishing a method for improving the efficiency and accuracy of gene knock-in using the Cpf1 non-retained end.

### SUMMARY

The purpose of the present invention is to provide a method for improving the efficiency and accuracy of gene knock-in using the Cpf1 non-retained end. This invention uses single or paired Cpf1 to generate free target DNA ends and free donor DNA ends, and combines with the complementary 5′-cohesive ends produced by Cpf1 to achieve more efficient and more accurate gene knock-in based on c-NHEJ, solving the problem of stably achieving efficient and accurate NHEJ repair-based gene knock-in using the 5′-cohesive ends with 5-nt overhangs induced by Cpf1; this NHEJ-mediated gene knock-in strategy will also provide a new technical option for accurate and efficient gene knock-in in non-dividing cells, overcoming the barrier that non-dividing cells cannot use HR for gene knock-in, and resolving the feasibility issue of gene editing technology for gene therapy involving gene knock-in in neurons and muscle cells.

The technical solution adopted by the present invention is as follows:

The present invention provides a method for improving the efficiency and accuracy of gene knock-in using the Cpf1 non-retained end, which is as follows: using single or paired Cpf1 to generate free target DNA ends and free donor DNA ends, and combining with the complementary 5′-cohesive ends produced by Cpf1 to achieve more efficient and more accurate gene knock-in based on c-NHEJ; if it is needed to improve the efficiency and accuracy of knocking the target gene into one end of the recipient genome (i.e., one end requires higher accuracy than the other in the ligation of the two ends of the recipient genome for target gene knock-in), the end of the recipient genome target that requires high ligation accuracy after cleavage shall be a free end, and at least ensure that the end of the donor target that needs to be correspondingly ligated is a free end after cleavage, with the free end of the donor complementarily ligated to the free end of the recipient genome requiring high accuracy; if it is necessary to improve the efficiency and accuracy of knocking the target gene into both ends of the recipient genome (i.e., both ends require high accuracy and efficiency in the ligation of the two ends of the recipient genome for target gene knock-in), both ends of the donor target after cleavage and the corresponding two ends of the recipient genome after cleavage shall be complementary free ends, and in this case, paired Cpf1 are required for both donor and recipient genome cleavage.

Preferably, the method for improving the efficiency and accuracy of N-terminal target gene knock-in is as follows: the PAM of recipient target gene for Cpf1 is located on W strand; the upstream PAM of the donor DNA precursor for Cpf1 is located on either W strand or C strand, while the downstream PAM of the donor DNA precursor for Cpf1 is located on C strand, and the corresponding 5′-cohesive ends of the recipient gene and the donor after Cpf1 cleavage are completely complementary.

Preferably, the method for improving the efficiency and accuracy of C-terminal target gene knock-in is as follows: the PAM of recipient target gene for Cpf1 is located on C strand; the upstream PAM of the donor DNA precursor for Cpf1 is located on W strand, while the downstream PAM of the donor DNA precursor for Cpf1 is located on either C strand or W strand, and the corresponding 5′-cohesive ends of the recipient gene and the donor after Cpf1 cleavage are completely complementary.

The method of the present invention for improving the efficiency and accuracy of N-terminal or C-terminal knock-in of a DNA fragment or gene tag using Cpf1 non-retained end in cells is carried out as follows:

1. Based on the requirement for N-terminal or C-terminal tag knock-in, select a testable Cpf1 target in the targeted gene according to the strand where the Cpf1 PAM is located. Since N-terminal tag knock-in requires precise ligation between the inserted tag and the junction of the downstream target gene, the downstream end (the second end) of the two ends of the DSB generated by Cpf1 target gene cleavage should be a free PAM-distal end, i.e., the PAM of the Cpf1 target in the target gene is located on Watson strand; if it is C-terminal tag knock-in, precise ligation between the inserted tag and the junction of the upstream target gene is required, therefore the upstream end (the first end) of the two ends of the DSB generated by Cpf1 target cleavage should be a free PAM-distal end, i.e., the PAM of the Cpf1 target in the target gene is located on Crick strand.

2. After selecting the Cpf1 target in the target gene, use T7E1 assay to test the cleavage efficiency of the selected target gene Cpf1 target in the target cells, and select the Cpf1 target that can be cleaved with high efficiency.

3. Design the donor DNA for N-terminal knock-in. The donor DNA precursor can be linear or loaded onto a plasmid. Both sides of the donor DNA precursor should contain a Cpf1 target site designed based on the target sequence of the target gene. The upstream PAM can be located on either Watson strand or Crick strand, but preferably on Watson strand, while the downstream PAM of must be located on Crick strand. This allows paired Cpf1-sgRNA to cleave the donor DNA precursor to generate donor DNA with two 5′-cohesive ends, each of which is completely in complementary to the corresponding end of the recipient gene, and the downstream end of the cleaved donor DNA is a free PAM-distal end; in addition, according to the design, its upstream 5′-cohesive end can be a Cpf1-retained PAM-proximal end, but is preferably a free PAM-distal end, which needs to be complementary to the upstream end of the Cpf1 target in the target gene, while the downstream 5′-cohesive end is a free PAM-distal end, which must be complementary to the downstream end of the Cpf1 target in the target gene.

4. Design the donor DNA for C-terminal knock-in. The donor DNA precursor can be linear or loaded onto a plasmid. Both sides of the donor DNA precursor should contain a Cpf1 target site designed based on the target sequence of the target gene. The upstream PAM target must be located on Watson strand, while the downstream PAM can be located on either Watson strand or Crick strand, but preferably on Crick strand. This allows paired Cpf1-sgRNA to cleave the donor DNA precursor to generate donor DNA with two 5′-cohesive ends, each of which is completely complementary to the corresponding end of the recipient gene, and the upstream end of the cleaved donor DNA is a free PAM-distal end. According to the design, after paired Cpf1-sgRNA cleaving the donor DNA precursor to generate donor DNA with two 5′-cohesive ends, the upstream 5′-cohesive end should be a free PAM-distal end, which needs to be completely complementary to the upstream end of the Cpf1 target in the target gene, while the downstream 5′-cohesive end can be a Cpf1-retained PAM-proximal end, but is preferably a free PAM-distal end, which should be completely complementary to the downstream end of the Cpf1 target in the target gene.

5. Specific Operation Methods is as follows:

(1) According to the tag knock-in position of the target gene, select a testable Cpf1 target of the target gene based on the strand where the Cpf1 PAM is located. For N-terminal tag knock-in, select a Cpf1 target around the start codon of the target gene according to the PAM sequence on the Watson strand, and design and construct Cpf1 sgRNAs and their expression plasmids. For C-terminal tag knock-in, select a Cpf1 target near the stop codon of the target gene according to the PAM sequence on the Crick strand (with the cleavage site located before the stop codon), and design and construct Cpf1 sgRNAs and their expression plasmids.

(2) To select the optimal Cpf1 targets for N-terminal knock-in and C-terminal knock-in of the target gene, transfect the constructed Cpf1 gRNA expression plasmids into the target cells together with the Cpf1 expression plasmid. Extract the cellular genome 72 hours after transfection, PCR-amplify the DNA fragment containing the Cpf1 cleavage target junction sequence, perform restriction enzyme digestion detection on the PCR product using the T7E1 kit, and select the Cpf1 targets with high-efficiency cleavage for subsequent tag knock-in of the target gene.

(3) Design and construct the donor DNA precursor, which can be linear or constructed into a plasmid. The design of the donor DNA precursor for N-terminal knock-in shall meet the following requirements: the two sides of the donor DNA precursor should contain Cpf1 targets designed according to the target gene's target sequence; the PAM of the upstream target can be located on either the Watson strand or the Crick strand, but it is preferable to be on the Watson strand, while the PAM of the downstream target must be on the Crick strand. This is to ensure that after the donor DNA precursor is cleaved by the paired Cpf1-sgRNA, the generated donor DNA carries two 5′-cohesive ends. Furthermore, the upstream 5′-cohesive end can be the Cpf1-retained PAM-proximal end, but it is preferable to be the free PAM-distal end, and it must be completely complementary to the upstream end of the target gene's Cpf1 target; the downstream 5′-cohesive end is the free PAM-distal end and must be completely complementary to the downstream end of the target gene's Cpf1 target. The design of the donor DNA precursor for C-terminal knock-in shall meet the following requirements: the two sides of the donor DNA precursor should contain Cpf1 targets designed according to the target gene's target sequence; the PAM of the upstream target must be on the Watson strand, the PAM of the downstream target can be on either the Watson strand or the Crick strand, but it is preferable to be on the Crick strand. In addition, after the donor DNA precursor is cleaved by the paired Cpf1-sgRNA, the generated donor DNA carries two 5′-cohesive ends; the upstream 5′-cohesive end should be the free PAM-distal end and must be completely complementary to the upstream end of the target gene's Cpf1 target, while the downstream 5′-cohesive end can be the Cpf1-retained PAM-proximal end, but it is preferable to be the free PAM-distal end and should be completely complementary to the downstream end of the target gene's Cpf1 target.

(4) Analysis and verification of N-terminal or C-terminal tag knock-in of the target gene. Transfect sgRNA plasmids targeting the target gene's target sites and donor precursor's target sites, Cpf1, and the donor precursor (either linear or plasmid-loaded) into cells. After 72 hours of transfection, perform dilution passaging and plating. When single colonies grow, pick single-cell clones, and perform PCR amplification of genomic DNA from single-cell clones targeting the knock-in gene. Based on the PCR products and their Sanger sequencing results, analyze the junction sequence of the knock-in gene to obtain single-cell clones with precise gene knock-in, and verify them via methods such as Western blotting. If the knock-in tag can be directly used for screening (e.g., the knock-in gene is a fluorescent protein gene), sort fluorescent protein gene-positive cells by flow cytometry 72 hours after transfection, then culture the fluorescent protein gene-positive cells to obtain single-cell clones.

The method for improving the efficiency and accuracy of knocking in a DNA fragment or gene using Cpf1 non-retained end of the present invention is carried out as follows:

1. Based on the requirement for the genomic target of DNA fragment or gene knock-in, identify two adjacent testable Cpf1 target sites of the genomic target. Since the knock-in of a DNA fragment or gene in this application requires precise ligation at both ends, the PAM of the upstream target shall be located on Crick strand, and the PAM of the downstream target shall be located on Watson strand. When Cpf1 cleaves these two paired Cpf1 targets simultaneously, it will delete the intervening sequence between the paired Cpf1 targets and generate two free PAM-distal ends in the genome, thereby enabling precise and efficient knock-in of a DNA fragment or gene through the NHEJ pathway.

2. Use target-specific PCR amplification to test the efficiency of Cpf1 in simultaneously cleaving the paired Cpf1 targets in the target cells, and select the paired target sites that can be cleaved simultaneously with high efficiency.

3. Design the donor DNA precursor for DNA fragment or gene knock-in. The donor DNA precursor can be linear or loaded onto a plasmid. Both sides of the donor DNA precursor contain two Cpf1 targets, the PAM of the upstream Cpf1 target is located on Watson strand, while the PAM of the downstream is located on Crick strand. Paired Cpf1-sgRNA cleaves the donor DNA precursor to generate donor DNA with two 5′-cohesive ends, both of which are free PAM-distal ends; the upstream 5′-cohesive end shall be completely complementary to the upstream free end of the genomic Cpf1 target, and the downstream 5′-cohesive end shall be completely complementary to the downstream free end of the genomic Cpf1 target.

### 4. Specific Operation Methods:

(1) Based on the genomic target sequence for DNA fragment or gene knock-in, select a testable paired genomic Cpf1 target, and design and construct Cpf1 sgRNAs and their expression plasmids. To achieve precise ligation of the DNA fragment or gene to be accurately knocked in using the free ends at the genomic target, the PAM of the upstream target in the paired genomic Cpf1 targets must be located on Crick strand, and the PAM of the downstream target must be located on Watson strand.

(2) To screen the optimal paired genomic Cpf1 targets for DNA fragment or gene knock-in, transfect the constructed paired Cpf1 gRNA expression plasmids into the target cells together with the Cpf1 expression plasmid. 72 hours after transfection, extract the cellular genomic DNA, and perform PCR amplification of the paired Cpf1 cleavage target sequences. Analyze the PCR amplification products by DNA gel electrophoresis, evaluate the efficiency of simultaneous cleavage of the targets by the paired Cpf1 based on the deletion efficiency of the intervening sequence, and select targets that can be cleaved simultaneously with high efficiency by the paired Cpf1 for subsequent DNA fragment or gene knock-in.

(3) Design and construct the donor DNA precursor, which can be linear or loaded onto a plasmid. Design the Cpf1 targets on both sides of the donor DNA precursor based on the sequence of the selected optimal paired genomic Cpf1 targets. The PAM of the upstream target in the donor DNA precursor is located on Watson strand, and the PAM of the downstream target is located on Crick strand, so that paired Cpf1-sgRNA cleaves the donor DNA precursor to generate donor DNA with two 5′-cohesive ends that are free PAM-distal ends. The upstream end must be completely complementary to the upstream free end of the upstream genomic Cpf1 target, and the downstream end must be completely complementary to the downstream free end of the downstream genomic Cpf1 target. Moreover, when designing the Cpf1 targets on both sides of the donor DNA precursor, except for the overhanging bases of the 5′-cohesive ends of the donor DNA, which are fixed due to complementarity requirements, other sequences of the targets can be selected from the optimal universal target sequences for Cpf1 cleavage.

(4) Analysis and verification of DNA fragment or gene knock-in. Transfect the target cells with the paired Cpf1 sgRNA plasmids (targeting the genomic target of interest and the donor precursor targets), Cpf1 expression plasmid, and donor precursor (linear or plasmid-loaded). 72 hours after transfection, perform dilution passage and plating. When single clones grow, pick single-cell clones. If the knocked-in gene can be directly used for screening (e.g., the knocked-in gene is a resistance gene or a fluorescent protein gene), obtain single-cell clones through resistance screening or sort fluorescent protein-positive cells by flow cytometry 72 hours after transfection, then culture the fluorescent protein-positive cells to obtain single-cell clones. Subsequently, perform PCR amplification of the genomic DNA of the single-cell clones targeting the knocked-in DNA fragment or gene and its junction. Based on the PCR products and their Sanger sequencing results, analyze the sequence of the knocked-in DNA fragment or gene and its junction to obtain single-cell clones with accurate knock-in, and verify them by methods such as Western blot, immunofluorescence microscopy, or flow cytometry.

The design scheme of the present invention for directionally generating complementary free 5′-cohesive ends between intracellular genomic targets and exogenous donor DNA targets using Cpf1 consists of four parts:

1. NHEJ-competent target cells, to enable precise knock-in of donor DNA into the genomic targets of the target cells via NHEJ.

2. Cpf1 nuclease and its associated sgRNAs, used for site-specific cleavage of genomic target DNA and donor DNA precursors in target cells, generating free 5′-cohesive ends with directionally complementary 5-nt overhangs between the two. After cleaving the DNA target, Cpf1 remains bound to the PAM-proximal end and releases the PAM-distal end, producing a free PAM-distal end. We found that the Cpf1 free end is more susceptible to binding and protection by the NHEJ core factor KU70/KU80, thereby achieving more efficient and more accurate repair via c-NHEJ. In contrast, the Cpf1-retained end hinders the binding of KU70/KU80 and thus lacks such protection, leading to increased susceptibility of this end to processing and higher likelihood of base insertions and deletions. This part uses this characteristic of Cpf1 nuclease and its associated sgRNAs, together with the complementarity of the 5′-cohesive ends generated by Cpf1, to facilitate efficient and accurate gene knock-in based on c-NHEJ. Cpf1 nuclease and its associated sgRNAs are provided in the form of plasmids, but other usable forms (such as RNA or protein) are not excluded. Cpf1 nucleases include but are not limited to Lachnospiraceae bacterium ND2006 Cpf1 (LbCpf1), Acidaminococcus sp. BV3L6 Cpf1 (AsCpf1), and Francisella novicida Cpf1 (FnCpf1).

3. Selection of Cpf1 genomic targets. Based on the knock-in position of the genomic target and the PAM sequence of Cpf1, single or paired Cpf1 genomic cleavage targets are selected, with the following requirements: (1) If a single Cpf1 genomic target is selected and one end requires higher accuracy than the other in the ligation of the two ends of the gene knock-in, at least ensure that the free end of the genomic target after cleavage is at the corresponding ligation position; (2) If adjacent paired Cpf1 genomic targets are selected, both ends of the excised intervening sequence shall be PAM-proximal ends, while both ends of the remaining genomic knock-in target shall be free PAM-distal ends. That is, the upstream PAM in the paired Cpf1 genomic targets must be located on Crick strand, and the downstream PAM must be located on Watson strand.

4. Design of Cpf1 cleavage targets for donor DNA. The donor DNA refers to the DNA fragment or gene to be knocked in. After confirming the donor DNA, a linear donor DNA precursor or donor DNA precursor plasmid containing paired Cpf1 targets at both ends of the donor DNA is designed and constructed based on the localization of the free ends of the Cpf1-cleaved genomic target and the base composition of the 5′-cohesive ends. According to this design, cleavage of the donor DNA precursor targets by paired Cpf1 will generate linear donor DNA with two 5′-cohesive ends. These two 5′-cohesive ends are directionally complementary to the two ends of the single Cpf1 or paired Cpf1 genomic knock-in target, respectively, and are free PAM-distal ends. That is, in the Cpf1 targets at both ends of the donor DNA before cleavage, the PAM of the upstream Cpf1 target must be located on Watson strand and the PAM of the downstream Cpf1 target must be located on Crick strand, or at least ensure that the end requiring high ligation accuracy and efficiency is a free PAM-distal end.

Compared with the prior art, the beneficial effects of the present invention are mainly reflected in the following aspects:

1. The method of the present invention enables more efficient and more accurate NHEJ repair, with efficiency improved by nearly two times and accuracy improved from 0% to 100%. The present invention uses the fact that the Cpf1-free end is bound and protected by the NHEJ core factor KU70/KU80, thereby achieving more efficient and more accurate ligation via NHEJ, laying a foundation for improving the efficiency and accuracy of NHEJ-based gene knock-in using the Cpf1-free end.

2. The NHEJ-based N-terminal and C-terminal DNA fragment or gene knock-in of the present invention is more efficient and more accurate, including N-terminal and C-terminal gene tag knock-in. HR-mediated N-terminal and C-terminal DNA fragment or gene knock-in has low efficiency, while CRISPR/Cas9-based NHEJ-mediated N-terminal and C-terminal DNA fragment or gene knock-in lacks sufficient accuracy and may result in inversion of the inserted DNA fragment or gene. In contrast, the present invention improves both the efficiency and accuracy of N-terminal and C-terminal DNA fragment or gene knock-in without inversion of the inserted DNA fragment or gene.

3. The NHEJ-based DNA fragment or gene knock-in of the present invention is more efficient and more accurate, enabling gene knock-in or gene correction. HR-mediated DNA fragment or gene knock-in has low efficiency, while CRISPR/Cas9-based NHEJ-mediated DNA fragment or gene knock-in lacks sufficient accuracy and may result in inversion of the knocked-in DNA fragment or gene. The present invention improves both the efficiency and accuracy of DNA fragment or gene knock-in without inversion of the inserted DNA fragment or gene, and thus has greater potential in practical applications (including gene therapy and plant variety improvement).

4. The present invention is applicable to non-dividing cells. HR-mediated gene knock-in is not suitable for non-dividing cells (such as neurons and muscle cells), while the gene knock-in technology and method of the present invention use the NHEJ pathway that occurs at all stages of the cell cycle. It is not only applicable to dividing cells but also can be used in non-dividing cells that only undergo the NHEJ pathway, providing a new strategy for gene therapy of neurodegenerative diseases and genetic muscular dystrophy.

### BRIEF DESCRIPTION OF THE DRAWINGS

FIGS. 1A-1B are the effect of end retention asymmetry on KU80 end binding after Cpf1 cleaving DNA targets in the present invention. FIG. 1A represents a schematic diagram of end retention asymmetry after Cpf1-sgRNA cleaves DNA targets and its effect on NHEJ repair efficiency and accuracy. The displayed DNA target is a representative sequence, and the underlined 5′-CTTG-3′ is the Cpf1 target PAM. Cpf1 has an N-terminal HA tag, and gC is its associated sgRNA. After Cpf1 cleaves the DNA target, the Cpf1-free end (i.e., the PAM-distal end) is susceptible to binding and protection by KU70/KU80, thereby utilizing c-NHEJ for repair with higher efficiency and accuracy; the Cpf1-retained end (i.e., the PAM-proximal end) hinders the binding of KU70/KU80 and thus lacks such protection, which leads to a higher likelihood of base insertions and deletions. FIG. 1B represents the result of a chromatin immunoprecipitation (ChIP) assay verifying that the Cpf1-free end is more likely to bind KU70/KU80.

FIGS. 2A-2C are the effect of end retention asymmetry on NHEJ repair end processing after Cpf1 cleaving DNA targets in the present invention. FIG. 2A represents a schematic diagram of the effect of end retention characteristics on NHEJ repair end processing after SpCas9 and LbCpf1 cleave DNA targets. The effect of dual-end retention of SpCas9 on end processing is symmetrical, while the effect of end retention asymmetry of LbCpf1 on end processing is asymmetrical. FIG. 2B represents the effect of end retention characteristics of SpCas9 and LbCpf1 on base deletion length during NHEJ repair after cleaving DNA targets. The dual-end retention of SpCas9 has similar effects on the base deletion length at both ends, whereas LbCpf1 retention at the PAM-proximal end and release at the PAM-distal end results in longer base deletion length at the PAM-proximal end. The black lines and values in the dot plot represent the average deletion length. FIG. 2C represents the effect of end retention characteristics of SpCas9 and LbCpf1 on the ratio and difference between the average base deletion lengths at the PAM-distal end and PAM-proximal end after cleaving DNA targets. * indicates a statistical P-value<0.005. n represents the number of tested gene targets.

FIGS. 3A-3B are the effect of Cpf1 end retention asymmetry on NHEJ repair depending on c-NHEJ activity in the present invention. LbCpf1-g5c cleaves the DNA target at the Rosa26 locus in mouse embryonic stem cells, inducing NHEJ. FIG. 3A represents the effect of KU80 knockout on base deletion length by neutralizing LbCpf1 end retention asymmetry. In normal KU80 cells, LbCpf1 end retention asymmetry inhibits base deletion at the LbCpf1-free end (i.e., the PAM-distal end); however, after KU80 knockout (in KU80″ cells), the base deletion length at the LbCpf1-free end is comparable to that at the LbCpf1-retained end (i.e., the PAM-proximal end). The black lines and values in the dot plot represent the average deletion length. FIG. 3B represents that the knockout of XRCC4 neutralizes the effect of LbCpf1 end retention asymmetry on the length of base deletions. In normal XRCC4 cells, LbCpf1 end retention asymmetry inhibits base deletion at the LbCpf1-free end (i.e., the PAM-distal end); however, in XRCC4^{-/-} cells, the base deletion length at the LbCpf1-free end is comparable to that at the LbCpf1-retained end (i.e., the PAM-proximal end). The black lines and values in the dot plot represent the average deletion length.

FIGS. 4A-4C are the effect of end retention asymmetry on c-NHEJ repair accuracy after paired Cpf1 cleaving DNA targets in the present invention. FIG. 4A represents a schematic diagram of end retention asymmetry of paired Cpf1 after cleaving DNA targets. Cpf1 PAM on Watson strand is denoted as W. Cpf1 PAM on Crick strand is denoted as C. Among them, the C/W PAM combination deletes the intervening sequence containing the PAM-proximal end (i.e., the Cpf1-retained end) at both ends, generating two Cpf1-free ends to be ligated with each other; the W/C combination generates two Cpf1-retained ends to be ligated with each other; the W/W and C/C combinations each generate one Cpf1-retained end and one Cpf1-free end to be ligated with each other. FIG. 4B represents the ligation accuracy at both ends induced by simultaneous cleavage of DNA targets by LbCpf1 in each of the four combinations. Each symbol in the plot represents the accuracy of one genomic target. The C/W combination induces the highest accuracy (close to 50%), which is much higher than the other three combinations (all approximately 5%). n represents the number of tested genomic target for each combination. FIG. 4C represents the average length of additional deletions at the two ligation ends after LbCpf1 simultaneously cleaves the DNA targets and deletes the intervening sequence in each of the four combinations. Each symbol in the plot represents the average additional deletion length of one genomic target for a paired PAM combination. The average value of the average additional deletion lengths of 9 paired targets induced by the C/W combination is less than 5 bp, which is lower than that of the other three combinations (all greater than 6 bp). n represents the number of tested genomic target for each combination.

FIGS. 5A-5C are the use of Cpf1 end retention asymmetry to affect the efficiency of translocation ligation in the present invention. FIG. 5A represents a schematic diagram of translocation induced by the combination of LbCpf1 and SpCas9. SpCas9-g10 and LbCpf1-g2 cleave targets on chromosome 3 and chromosome 8, respectively, and the broken ends can undergo two types of direct ligation (i.e., 1 and 2) and four different combinations of translocation (3, 4, 5, and 6). These products can be detected by PCR: paired primers F3/R3 detect direct ligation product 1, F2/R2 detect direct ligation product 2, paired primers F3/R2 detect translocation product 3, paired primers F2/R3 detect translocation product 4, paired primers F3/F2 detect translocation product 5, and paired primers R3/R2 detect translocation product 6. FIG. 5B represents the PCR products of the two direct ligation products and four translocation products. The PCR product of GAPDH serves as the internal reference. The blank control refers to cells without LbCpf1 and SpCas9 expression, i.e., no target cleavage. The PCR products of translocations 3, 4, 5, and 6 were further confirmed by Sanger sequencing. FIG. 5C represents the quantitative analysis of the PCR products of the two direct ligation products and four translocation products. The ordinate shows the ratio of the gray value of the PCR products of the two direct ligation products and four translocation products to the gray value of the PCR product of the internal reference GAPDH.

FIGS. 6A-6D are the use of Cpf1-free end to improve the efficiency of N-terminal accurate gene knock-in in the present invention. FIG. 6A represents a schematic diagram of the simulated site and knock-in design for precise N-terminal gene knock-in. The intracellular genomic target is intron 10 of human HNRNPA1; after cleavage by LbCpf1-gW, an upstream LbCpf1-retained end and a downstream LbCpf1-free end are generated. The donor is the pPGK-RFP-pA expression cassette. In the donor precursor (donor plasmid), the donor contains LbCpf1 targets at both ends, designed into four combinations: W/W, W/C, C/C, and C/W. Cleavage of the W/W and W/C combinations generates a donor with an upstream free end, while cleavage of the C/C and C/W combinations generates a donor with an upstream retained end. The 5-nt 5′-cohesive ends at both ends of the donor are directionally complementary to the two 5-nt 5′-cohesive ligation ends of the genomic target for gene knock-in, respectively. FIG. 6B represents the target gene knock-in efficiency of the four donor combinations. The W/C combination exhibits the highest target gene knock-in efficiency. FIG. 6C represents the PCR amplification of the C-terminal (5′-junction) and N-terminal (3′-junction) target gene knock-in junction sequences for the four donor combinations. No clear and specific PCR bands were observed in the non-cleaved genomic target (i.e., the U6 control group), while clear and specific PCR bands were observed in the experimental group. For the 5′-junction, the W/W and W/C combinations produce the most prominent PCR bands; for the 3′-junction, the W/C combination produces the most prominent PCR band. FIG. 6D represents the accuracy detection results of C-terminal (5′-junction) and N-terminal (3′-junction) target gene knock-in for the four donor combinations. The W/C and C/C combinations exhibit significantly higher accuracy in N-terminal (3′-junction) target gene knock-in than the W/W and C/W combinations. The efficiency and accuracy of the W/C combination demonstrate that the Cpf1-free end can be used to achieve more efficient and more accurate N-terminal target gene knock-in.

FIGS. 7A-7D are the use of Cpf1-free end to improve the efficiency of C-terminal accurate gene knock-in in the present invention. FIG. 7A represents a schematic diagram of the simulated site and knock-in design for precise C-terminal gene knock-in. The intracellular genomic target is the intronic site between exon 1 and exon 2 of human AAVS1; after cleavage by LbCpf1-gC, an upstream LbCpf1-free end and a downstream LbCpf1-retained end are generated. The donor is the pPGK-RFP-pA expression cassette. In the donor precursor (donor plasmid), the donor contains LbCpf1 targets at both ends, designed into four combinations: W/W, W/C, C/C, and C/W. Cleavage of the W/W and W/C combinations generates a donor with an upstream free end, while cleavage of the C/C and C/W combinations generates a donor with an upstream retained end. The 5-nt 5′-cohesive ends at both ends of the donor are directionally complementary to the two 5-nt 5′-cohesive ligation ends of the genomic target for gene knock-in, respectively. FIG. 7B represents the target gene knock-in efficiency of the four donor combinations. The W/C and C/C combinations exhibit the highest target gene knock-in efficiency. FIG. 7C represents the PCR amplification of the C-terminal (5′-junction) and N-terminal (3′-junction) target gene knock-in junction sequences for the four donor combinations. No clear and specific PCR bands were observed in the non-cleaved genomic target (i.e., the U6 control group), while clear and specific PCR bands were observed in the experimental group. For the 5′-junction, the W/W and W/C combinations produce the most prominent PCR bands; for the 3′-junction, the W/W combination produces the most prominent PCR band. FIG. 7D represents the accuracy detection results of C-terminal (5′-junction) and N-terminal (3′-junction) target gene knock-in for the four donor combinations. The W/C and C/C combinations exhibit significantly higher accuracy in C-terminal (5′-junction) target gene knock-in than the W/W and C/W combinations. The efficiency and accuracy of the W/C combination demonstrate that the Cpf1-free end can be used to achieve more efficient and more accurate C-terminal target gene knock-in.

FIGS. 8A-8F are the use of Cpf1-free end to improve the efficiency of CANX-RFP fusion via dual-end accurate gene knock-in in the present invention. FIG. 8A represents a schematic diagram of the simulated human CANX target and knock-in design for dual-end accurate gene knock-in. LbCpf1-gC and LbCpf1-gW cleave the last exon of CANX (i.e., exon 21) in pairs, delete the exon 21 intervening sequence with LbCpf1-retained ends at both ends, and generate gene knock-in ligation ends with LbCpf1-free ends at both the upstream and downstream. The donor is the exon 21-linker-RFP gene cassette. In the donor precursor (donor plasmid), the donor contains LbCpf1 targets at both ends, designed as the W/C combination (i.e., gWd/gCd), and only this combination can be cleaved to generate a donor with both upstream and downstream free ends. The 5-nt 5′-cohesive ends at both ends of the donor are directionally complementary to the two 5-nt 5′-cohesive ligation ends of the genomic target for gene knock-in, respectively. FIG. 8B represents the cleavage efficiency of selected C/W combinations in paired cleavage of the last exon of CANX (i.e., exon 21). PCR amplification of CANX targets after paired cleavage shows that the C1/W1 combination exhibits significantly higher efficiency in simultaneous target cleavage than C3/W1; therefore, the C1/W1 combination was selected for the dual-end accurate gene knock-in experiment to generate CANX-RFP fusion. FIG. 8C represents the frequency of RFP cells generated by dual-end gene knock-in. The sgRNAs of the paired C1/W1 combination targeting CANX targets, the sgRNAs of the gWd/gCd combination targeting the donor plasmid, the donor plasmid, and the LbCpf1 expression plasmid were co-transfected into HEK293T cells. 10 days after transfection, the frequency of RFP cells generated by dual-end gene knock-in was measured. The frequency of RFP cells generated by gene knock-in is significantly higher than the background without LbCpf1 target cleavage (i.e., the control group EV and the control group U6).

FIG. 8D represents the accuracy detection results of the dual-end gene knock-in junctions. After gene knock-in, PCR was performed to amplify the dual-end gene knock-in junctions. The results show that target gene knock-in only occurred in the experimental group; moreover, Sanger sequencing indicates that the accuracy of the 5′-junction and 3′-junction is as high as 95% (i.e., 20 out of 21 cases are accurate) and 100% (i.e., 32 out of 32 cases are accurate), respectively. FIG. 8E represents the localization result of the RFP fusion protein. After sorting the RFP cells generated by gene knock-in and staining with DAPI, observation by fluorescence microscopy shows that the RFP fusion protein is localized in the cytoplasm, which is consistent with the cytoplasmic localization of CANX. FIG. 8F represents the expression of the CANX-RFP fusion protein. Western blot shows that the CANX-RFP fusion protein is expressed in RFP cells, with β-actin as the internal reference.

FIGS. 9A-9F are the use of Cpf1-free end to improve the efficiency of PCNA-RFP fusion via dual-end accurate gene knock-in in the present invention. FIG. 9A represents a schematic diagram of the simulated human PCNA target and knock-in design for dual-end accurate gene knock-in. LbCpf1-gC and LbCpf1-gW cleave the last exon of PCNA (i.e., exon 7) in pairs, delete the exon 7 intervening sequence with LbCpf1-retained ends at both ends, and generate gene knock-in ligation ends with LbCpf1-free ends at both the upstream and downstream. The donor is the exon 7-linker-RFP gene cassette. In the donor precursor (donor plasmid), the donor contains LbCpf1 targets at both ends, designed as the W/C combination (i.e., gWd/gCd), and only this combination can be cleaved to generate a donor with both upstream and downstream free ends. The 5-nt 5′-cohesive ends at both ends of the donor are directionally complementary to the two 5-nt 5′-cohesive ligation ends of the genomic target for gene knock-in, respectively. FIG. 9B represents the cleavage efficiency of selected C/W combinations in paired cleavage of the last exon of PCNA (i.e., exon 7). PCR amplification of PCNA targets after paired cleavage by four C/W combinations shows that the C1/W2 combination exhibits the optimal efficiency in simultaneous target cleavage; therefore, the C1/W2 combination was selected for the dual-end accurate gene knock-in experiment to generate PCNA-RFP fusion. FIG. 9C represents the frequency of RFP cells generated by dual-end gene knock-in. The sgRNAs of the paired C1/W2 combination targeting PCNA targets, the sgRNAs of the gWd/gCd combination targeting the donor plasmid, the donor plasmid, and the LbCpf1 expression plasmid were co-transfected into HEK293T cells. Ten days after transfection, the frequency of RFP cells generated by dual-end gene knock-in was measured. The frequency of RFP cells generated by gene knock-in is significantly higher than the background without LbCpf1 target cleavage (i.e., the control group EV and the control group U6). FIG. 9D represents the accuracy detection results of the dual-end gene knock-in junctions. After gene knock-in, PCR was performed to amplify the dual-end gene knock-in junctions. The results show that target gene knock-in only occurred in the experimental group; moreover, Sanger sequencing indicates that the accuracy of the 5′-junction and 3′-junction is 42% (i.e., 11 out of 26 cases are accurate) and 64% (i.e., 14 out of 22 cases are accurate), respectively. FIG. 9E represents the localization result of the RFP fusion protein. After sorting the RFP cells generated by gene knock-in and staining with DAPI, observation by fluorescence microscopy shows that the RFP fusion protein is localized in the cytoplasm, which is consistent with the cytoplasmic localization of PCNA. FIG. 9F represents the expression of the PCNA-RFP fusion protein. Western blot shows that the PCNA-RFP fusion protein is expressed in RFP cells, with CANX as the internal reference.

### DETAILED DESCRIPTION OF THE EMBODIMENTS

The present invention will be further described below with reference to the accompanying drawings and embodiments, but the present invention is not limited to the following Examples.

### Example 1: Cpf1 End Retention Asymmetry Affects NHEJ Efficiency and Accuracy

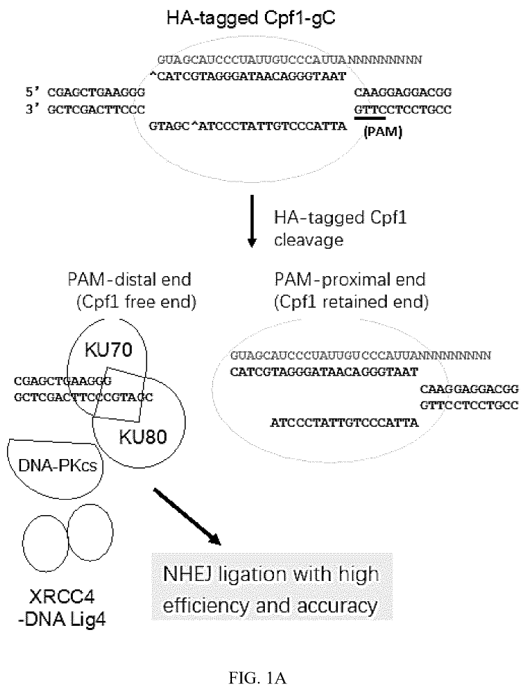

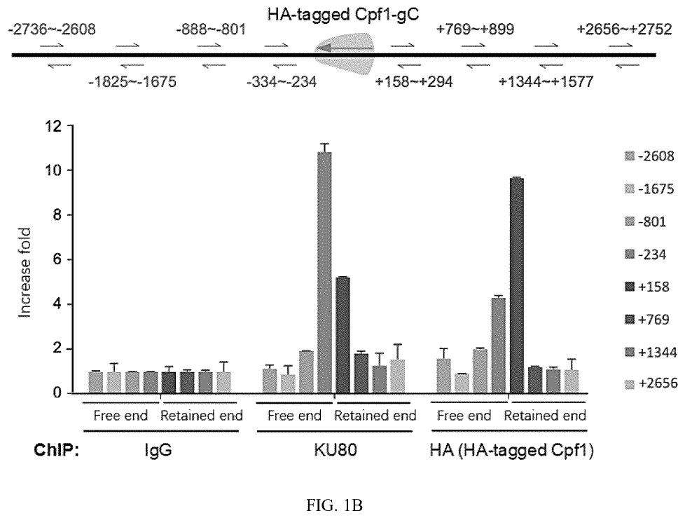

As illustrated in FIGS. 1A-5C of this Example, after Cpf1 cleaves DNA targets, the free PAM-distal end is more likely to bind to KU70/KU80 and undergo more efficient and more accurate c-NHEJ repair. In contrast, the Cpf1-retained PAM-proximal end hinders KU70/KU80 binding, making it prone to processing, which further leads to increased insertions, deletions, and longer deletion lengths during NHEJ repair. The specific embodiment is as follows:

1. Effect of End Retention Asymmetry on KU80 End Binding after Cpf1 Cleaves DNA Targets

KU70/KU80 typically binds to DNA ends as a heterodimer. To clarify the effect of end retention asymmetry on KU70/KU80 end binding after Cpf1 cleaves DNA targets, we used chromatin immunoprecipitation (ChIP) assays to detect the binding ability of Cpf1-retained end and the free end to KU70/KU80 after DNA targets are cleaved by Cpf1. The details are as follows:

(1) In mouse embryonic stem cells carrying an HR reporter system, a gRNA for the LbCpf1 target (i.e., gC) was designed and constructed in the I-SceI-GFP sequence of the HR reporter system (SEQ ID NO: 1; 389-406 bp is the I-SceI sequence, the rest represents the gene GFP encoding green fluorescent protein; 384-406 bp: CATCGTAGGGATAACAGGGTAAT (SEQ ID NO: 263) is gC targeting sequence). The spacer sequence of gC is 5′-ATTACCCTGTTATCCCTACGATG-3′ (SEQ ID NO: 264), with its PAM (5′-CTTG-3′) located on Crick strand (FIG. 1A). The aforementioned 5′-ATTACCCTGTTATCCCTACGATG-3′ (SEQ ID NO: 264) sequence was synthesized and ligated into the pU6-LbCpf1-crRNA vector (purchased from Addgene, Cat. No. 78957) to generate the gC expression plasmid. When Cpf1 (with an N-terminal HA tag) combines with gC to cleave the target, a double stranded break (DSB) with two ends is generated, where Cpf1 remains bound to the PAM-proximal end while releasing the PAM-distal end (FIG. 1A). It was hypothesized that the Cpf1-retained end would hinder the binding of KU70/KU80 (a core factor of NHEJ), whereas the Cpf1-free end would not be affected by such hindrance. Consequently, compared with the Cpf1-retained end, the Cpf1-free end is more likely to undergo efficient and accurate c-NHEJ repair dependent on KU70/KU80 (FIG. 1A).

(2) To verify the above hypothesis, Lipofectamine 2000 liposome transfection method was used to transfect the constructed LbCpf1 gC plasmid and the LbCpf1 expression plasmid into mouse embryonic stem cells carrying the HR reporter system. The transfection system (taking a 24-well plate as an example) is: each well contained 2×10⁵ cells; the total amount of transfected DNA was 0.5 μg including 0.25 μg LbCpf1 expression plasmid and 0.25 μg gC plasmid.

(3) ChIP assay: According to the design in Step 2, transfection was performed in 3 24-well plates (72 wells in total). At 24 hours post-transfection, the ChIP assay was conducted using the SimpleChIP® Plus Enzymatic Chromatin IP Kit (CST, Cat. No. #9003) following the manufacturer's instructions. First, chromatin supernatant was obtained using the kit, and then incubated with anti-Ku80 (CST, Cat. No. #2753), HA (Santa Cruz, Cat. No. #sc-7392), and non-specific IgG antibody for 12 hours. Subsequently, the ChIP products were purified according to the instructions of the SimpleChIP® Plus Enzymatic Chromatin IP Kit, yielding DNA products enriched by the IgG antibody, Ku80 antibody, and HA antibody.

(4) Quantitative real-time PCR (qPCR) amplification of ChIP products was performed using the primers corresponding to different positions of the genome as shown in Table 1. 20 μL qPCR system: 10 μL SYBR Green mix (Vazyme, Cat. No. Q111-02), 1 μL primer pair (10 μM), 2 μL purified DNA product, and double-distilled water to make up the volume to 20 μL. The PCR amplification conditions were: initial denaturation at 95° C. for 10 minutes, followed by 40 cycles of 95° C. for 15 seconds, 55° C. for 15 seconds, and 72° C. for 15 seconds. The increase fold of Ku80 and HA-LbCpf1 at each genomic position relative to the negative IgG background was calculated using the standard 2^{-ΔΔCt} method (FIG. 1B). ChIP results with the HA antibody showed that Cpf1 was indeed retained at the PAM-proximal end while releasing the PAM-distal end (FIG. 1B). Meanwhile, ChIP results with the KU80 antibody showed that the increase fold of genomic DNA targets by the KU80 antibody at the Cpf1-free end (i.e., PAM-distal end) was significantly higher than that at the Cpf1-retained PAM-proximal end (FIG. 1B). These results indicate that the free PAM-distal end generated after DNA targets are cleaved by Cpf1 is more likely to bind to KU70/KU80, whereas the Cpf1-retained PAM-proximal end hinders KU70/KU80 binding.

TABLE 1
[]Sequences of qRT-PCR primers

| | Primer Sequence | SEQ ID |
|---|---|---|
| PCR Primer Name | (5′-3′) | NO: |
| CHIP-HR-m2736-F | TGGCTGGCGTGGAAATATTC | 5 |
| CHIP-HR-m2608-R | CCGGTTTGGACTCAGAGTAT | 6 |
| CHIP-HR-m1825-F | CAGGCAGAAGTATGCAAAGC | 7 |
| CHIP-HR-m1675-R | CCTCGGCCTCTGCATAAATA | 8 |
| CHIP-HR-m888-F | TCTGGAGCATGCGCTTTAG | 9 |
| CHIP-HR-m801-R | TACCGGTGGATGTGGAATGT | 10 |
| CHIP-HR-m334-F | CTGGACGGCGACGTAAAC | 11 |
| CHIP-HR-m234-R | CGGTGGTGCAGATGAACTT | 12 |
| CHIP-HR-p158-F | AGCTCGCCGACCACTAC | 13 |
| CHIP-HR-p294-R | TCCAGCAGGACCATGTGAT | 14 |
| CHIP-HR-p769-F | GGACAAGACTTCCCACAGATT | 15 |
| CHIP-HR-p899-R | GGCGGATCACAAGCAATAATA | 16 |
| CHIP-HR-p1344-F | TCCACATTTGGGCCTATTCTC | 17 |
| CHIP-HR-p1577-R | AGACATCCACCTGAAACCATT | 18 |
| CHIP-HR-p2656-F | GGGGCTCTGAGATTTCATAAA | 19 |
| CHIP-HR-p2752-R | CCCAAATCTCATCAGAAAGG | 20 |

2. Effect of End Retention Asymmetry on NHEJ Repair End Processing after Cpf1 Cleaves DNA Targets

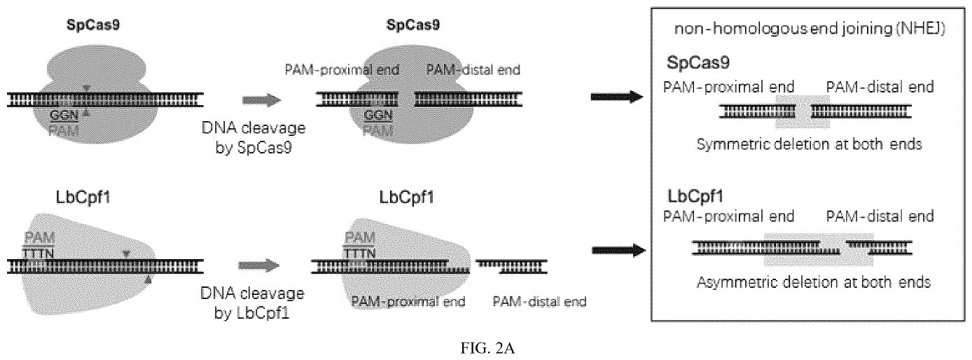

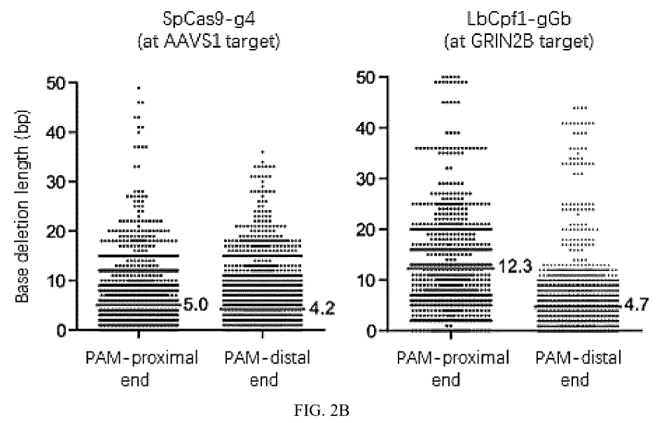

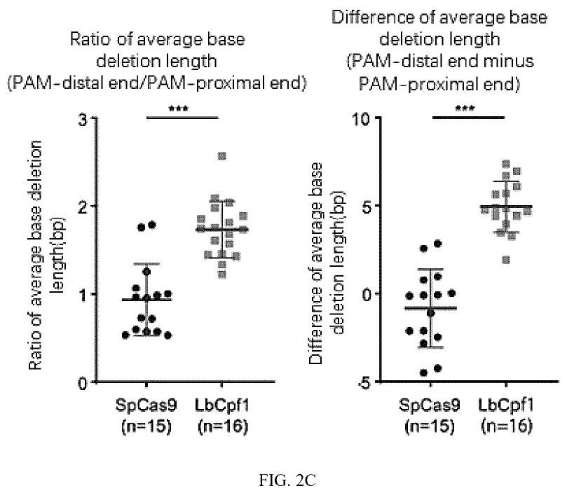

The end retention asymmetry after Cpf1 cleaves DNA targets affects KU70/KU80 binding; the Cpf1-free PAM-distal end is more likely to bind to KU70/KU80, while the Cpf1-retained PAM-proximal end hinders such binding. We hypothesized that KU70/KU80 binding would protect the free PAM-distal end from processing. Additionally, it would facilitate the recruitment of downstream NHEJ core factors DNA-PKcs and XRCC4/DNA ligase 4, accelerating NHEJ repair. Consequently, the deletion length at this end in NHEJ products should be relatively short. In contrast, the Cpf1-retained end, unprotected by KU70/KU80, is prone to processing, leading to a relatively longer deletion length in NHEJ products. In other words, this asymmetric binding of KU70/KU80 affects end processing during NHEJ repair, resulting in asymmetric deletions at the two ends in NHEJ products (FIG. 2A). In contrast, SpCas9 remains bound to both ends after cleaving DNA targets, exerting similar effects on KU70/KU80 binding at both ends; thus, the deletion lengths at the two ends in NHEJ products should show little difference (FIG. 2A). To verify this hypothesis, we analyzed the deletion lengths at the two ends of NHEJ repair products after SpCas9 and Cpf1 cleave DNA targets using deep sequencing of target PCR amplicons. The details are as follows:

(1) Based on the target sequence at the AAVS1 locus in HEK293 cells, an SpCas9 target sgRNA g4 was designed. The targeting sequence of g4 is 5′-AGACCCAATATCAGGAGACT-3′ (SEQ ID NO: 30), with its PAM (5′-AGG-3′) located on Crick strand. Based on the target sequence of the GRIN2B gene in HEK293 cells, an LbCpf1 target sgRNA gGb was designed. Its targeting sequence is 5′-TGATAAAGTGAAAAGAGCACTAA-3′ (SEQ ID NO: 40), with its PAM (5′-TTTG-3′) located on Crick strand. The targeting sequence of SpCas9 g4 (5′-AGACCCAATATCAGGAGACT-3′, SEQ ID NO: 30) was synthesized and ligated into the general px330-U6-Chimeric vector to generate the g4 expression plasmid. The targeting sequence of LbCpf1 gGb (5′-TGATAAAGTGAAAAGAGCACTAA-3′, SEQ ID NO: 40) was synthesized and ligated into the general pU6-LbCpf1-crRNA vector to generate the gGb expression plasmid. Both the px330-U6-Chimeric and pU6-LbCpf1-crRNA vectors were purchased from Addgene, with Cat. Nos. 42230 and 78957, respectively.

(2) Lipofectamine 2000 liposome transfection method was used to co-transfect HEK293 cells with: g4 expression plasmid and SpCas9 expression plasmid; gGb expression plasmid and LbCpf1 expression plasmid. The transfection system (taking a 24-well plate as an example) is: each well contained 1.4×10⁵ cells; the total amount of transfected DNA was 0.5 μg, including 0.25 μg of SpCas9 or LbCpf1 expression plasmid and 0.25 μg of sgRNA g4 or gGb expression plasmid.

(3) 72 hours after transfection, cellular genomic DNA was extracted, and sequences containing the target ligation junction were amplified by PCR. Through appropriate primer design, DNA fragments with PCR products less than 250 bp were obtained. The forward primer F and reverse primer R for amplifying the SpCas9-g4 target were 5′-TGGGACCACCTTATATTCCC-3′ (SEQ ID NO: 62) and 5′-CATCGTAAGCAAACCTTAGAG-3′ (SEQ ID NO: 265), respectively. The forward primer F and reverse primer R for amplifying the LbCpf1-gGb target were 5′-CTGGCGTTTGGACTCAGTTC-3′ (SEQ ID NO: 72) and 5′-CGCTGGTCTCAAGTCTCTTG-3′ (SEQ ID NO: 73), respectively.

(4) After PCR target amplification, the PCR products were subjected to deep sequencing to analyze the base deletion lengths at the PAM-proximal end and PAM-distal end. For the SpCas9-g4 cleavage target, the base deletion lengths at the two ends were similar, with average values of 5.0 bp and 4.2 bp, respectively. For the LbCpf1-gGb cleavage target, the base deletion lengths at the two ends differed significantly: the deletion length at the PAM-proximal end was 12.3 bp, while that at the PAM-distal end was 4.7 bp (FIG. 2B). These results initially indicate that, compared with SpCas9, the retention of LbCpf1 at the PAM-proximal end and its release at the PAM-distal end lead to a longer base deletion length at the PAM-proximal end.

(5) To further indicate that the retention of LbCpf1 at the PAM-proximal end and its release at the PAM-distal end cause asymmetric deletion lengths at the two ends, we designed 15 sgRNAs targeting SpCas9 sites (sequences shown in Table 2) and 16 sgRNAs targeting LbCpf1 sites (sequences shown in Table 3) based on genomic targets in mouse embryonic stem cells and human HEK293 cells, following the method described in Step (1). The targeting sequences of these 15 SpCas9-targeting sgRNAs and 16 LbCpf1-targeting sgRNAs were synthesized and ligated into the px330-U6-Chimeric vector and pU6-LbCpf1-crRNA vector, respectively, to generate sgRNA expression plasmids. According to the method in step (2) for transfection, the method in step (3) for PCR target amplification (the primer sequences for amplifying different SpCas9 targets are shown in Table 4, and the primer sequences for amplifying different Cpf1 targets are shown in Table 5), and the deep sequencing analysis in step (4). Based on the base deletion lengths at the PAM-proximal end and PAM-distal end, we calculated the ratio and difference between the average deletion length at the PAM-distal end and that at the PAM-proximal end (FIG. 2C). For SpCas9 cleavage targets, SpCas9 retention at two ends exerted similar effects on the base deletion lengths at the two ends, with the ratio and difference of average deletion lengths being approximately 1 and 0, respectively. For LbCpf1 cleavage targets, the retention of Cpf1 at the PAM-proximal end and its release at the PAM-distal end led to a longer base deletion length at the PAM-proximal end, with the ratio of average deletion lengths approaching 2 and the difference of average deletion lengths being approximately 5. The P-values for these differences were all less than 0.005. These results indicate that, unlike SpCas9, the free PAM-distal end generated after Cpf1 cleaves DNA targets is more likely to undergo efficient and accurate c-NHEJ repair, resulting in shorter deletions; in contrast, the Cpf1-retained PAM-proximal end is prone to processing, leading to longer deletions.

TABLE 2
[]Sequences of 15 sgRNAs for SpCas9 targets

| | | | SEQ | Strand |
|---|---|---|---|---|
| gRNA | | | ID | with |
| name | PAM | sgRNA Sequence | NO: | PAM |
| b1 | AGG | TGGATTACCCTGTTATCCCT | 21 | W |
| db2 | TGG | CTTGCTGATCATGTGAAGGA | 22 | C |
| dd3 | CGG | TTGAGCTCGAGATCTGAGTC | 23 | C |
| back-c1 | TGG | CTTCCGGCTCGTATGTTGTG | 24 | C |
| back-c2 | AGG | TGTGAGTTAGCTCACTCATT | 25 | C |
| back-w2 | CGG | ATTCCACACAACATACGAGC | 26 | W |
| 1-1 | AGG | GGATAACAGGGTAATCAAGG | 27 | W |
| 1-3 | GGG | AGGGCATCGTAGGGATAACA | 28 | W |
| AAVS1-g1 | GGG | TGTCCCCTCCACCCCACAGT | 29 | W |
| AAVS1-g4 | AGG | AGACCCAATATCAGGAGACT | 30 | C |
| HBB-g1 | AGG | TGGTATCAAGGTTACAAGAC | 31 | C |
| HBB-g3 | GGG | CACGTTCACCTTGCCCCACA | 32 | W |
| HBB-g4 | AGG | CATGGTGCATCTGACTCCTG | 33 | C |
| Rosa-g3 | AGG | CCTAAGAATGAGAAAGGCAA | 34 | W |
| Rosa-g5 | TGG | GATGCTGCAGCCCGCCCTGC | 35 | C |

TABLE 3
[]Sequences of 16 sgRNAs for LbCpf1 targets

| | | | SEQ | Strand |
|---|---|---|---|---|
| gRNA | | | ID | with |
| Name | PAM | sgRNA Sequence | NO: | PAM |
| gAAVS1- | TTTG | ACCCACTCCCCATTTCAACCAAA | 36 | C |
| Cpf1-1a | | | | |
| gAAVS1- | TTTC | TGTAAATCTGAAGCTTTCCCAAA | 37 | C |
| Cpf1-1c | | | | |
| gEMX1- | TTTG | CTGATTAGAATTCTACCCCTCTT | 38 | W |
| Cpf1-4b | | | | |
| gGRIN2B- | TTTA | CAGATTGTGTTGCCATTATTAGT | 39 | W |
| Cpf1-3a | | | | |
| gGRIN2B- | TTTG | TGATAAAGTGAAAAGAGCACTAA | 40 | C |
| Cpf1-3b | | | | |
| gGRIN2B- | TTTA | TAAGAAAAGCATTTTAAATATAA | 41 | W |
| Cpf1-7b | | | | |
| gGRIN2B- | TTTC | TAGCATATAGTATAATAAAAAAG | 42 | W |
| Cpf1-13a | | | | |
| gGRIN2B- | TTTC | ATCAGGCAAGTGTGGGTCCATTT | 43 | W |
| Cpf1-16a | | | | |
| gGRIN2B- | TTTG | CAAGAATCTATGTCTCTAAAATG | 44 | C |
| Cpf1-16b | | | | |
| gRUNX1- | TTTC | CTCAGTTCCTACCAGGACAGCTG | 45 | C |
| Cpf1-3a | | | | |
| gRUNX1- | TTTG | GAGAAGAGGGAGAGAAAACAGCT | 46 | W |
| Cpf1-3b | | | | |
| gRUNX1- | TTTC | ACCCTTGCCTTGCAGCAAAGAAA | 47 | C |
| Cpf1-6a | | | | |
| gRUNX1- | TTTG | ATGGGGGATTACACTGAATCTTT | 48 | W |
| Cpf1-10a | | | | |
| gR26- | TTTC | ACAAATCACAAAACCAACATTTG | 49 | C |
| Cpf1-5c | | | | |
| gR26- | TTTA | GAACCAAGGGTCTTAGAGTTTTA | 50 | W |
| Cpf1-6d | | | | |
| gR26- | TTTC | ACATTAAGTACAAATGTTTAACA | 51 | C |
| Cpf1-8c | | | | |

TABLE 4
Sequences of 15 primers for SpCas9 targets

| PCR Primer | | |
|---|---|---|
| Name | Primer Sequence (5′-3′) | SEQ ID NO: |
| b1-F | CCGCGCTGTTCTCCTCTTCC | 52 |
| b1-R | ATCGCCCTCGCCCTCGCCGG | 53 |
| db2-F | CCGCGCTGTTCTCCTCTTCC | 52 |
| db2-R | ATCGCCCTCGCCCTCGCCGG | 53 |
| dd3-F | TCCCGAGGCCCGGCATTCTG | 54 |
| dd3-R | GCCGTCCAGCTCGACCAGGA | 55 |
| back-c1-F | GGGCTCGATCCTCTAGTTGG | 56 |
| back-c1-R | CACGACAGGTTTCCCGACTG | 57 |
| back-c2-F | GGGCTCGATCCTCTAGTTGG | 56 |
| back-c2-R | CACGACAGGTTTCCCGACTG | 57 |
| back-w2-F | GGGCTCGATCCTCTAGTTGG | 56 |
| back-w2-R | CACGACAGGTTTCCCGACTG | 57 |
| 1-1-F | TCCGCCATGCCCGAAGGCTA | 58 |
| 1-1-R | TCCCTCGATGTTGTGGCGGA | 59 |
| 1-3-F | CGAAGGCTACGTCCAGGAGC | 60 |
| 1-3-R | TGTGGCGGATCTTGAAGTTC | 61 |
| AAVS1-g1-F | TGGGACCACCTTATATTCCC | 62 |
| AAVS1-g1-R | CATCGTAAGCAAACCTTAGA | 63 |
| AAVS1-g4-F | TGGGACCACCTTATATTCCC | 62 |
| AAVS1-g4-R | CATCGTAAGCAAACCTTAGA | 63 |
| HBB-g1-F | GGGTGGGAAAATAGACCAAT | 64 |
| HBB-g1-R | CAGAGCCATCTATTGCTTAC | 65 |
| HBB-g3-F | GGGTGGGAAAATAGACCAAT | 64 |
| HBB-g3-R | CAGAGCCATCTATTGCTTAC | 65 |
| HBB-g4-F | GGGTGGGAAAATAGACCAAT | 64 |
| HBB-g4-R | CAGAGCCATCTATTGCTTAC | 65 |
| Rosa-g3-F | GCTGGTGAAGACGTTACACA | 66 |
| Rosa-g3-R | AAGCCAAGATCCTAGGTAAA | 67 |
| Rosa-g5-F | GCTGGTGAAGACGTTACACA | 66 |
| Rosa-g5-R | AAGCCAAGATCCTAGGTAAA | 67 |

TABLE 5
[]Sequences of 16 primers for LbCpf1 targets

| | | SEQ |
|---|---|---|
| | Primer Sequence | ID |
| PCR Primer Name | (5′-3′) | NO: |
| gAAVS1-Cpf1-1a-F | GCCAGGCTGAAAGGATAGG | 68 |
| gAAVS1-Cpf1-1a-R | AGAAAAAGCACTGGCGAATC | 69 |
| gAAVS1-Cpf1-1c-F | GCCAGGCTGAAAGGATAGG | 68 |
| gAAVS1-Cpf1-1c-R | AGAAAAAGCACTGGCGAATC | 69 |
| gEMX1-Cpf1-4b-F | CCCTCTTGCAAAAGTGTCCA | 70 |
| gEMX1-Cpf1-4b-R | TTTACGTCTGCCTCCTCACC | 71 |
| gGRIN2B-Cpf1-3a-F | CTGGCGTTTGGACTCAGTTC | 72 |
| gGRIN2B-Cpf1-3a-R | CGCTGGTCTCAAGTCTCTTG | 73 |
| gGRIN2B-Cpf1-3b-F | CTGGCGTTTGGACTCAGTTC | 72 |
| gGRIN2B-Cpf1-3b-R | CGCTGGTCTCAAGTCTCTTG | 73 |
| gGRIN2B-Cpf1-7b-F | TGGATCATGTAGTTGCAGGGT | 74 |
| gGRIN2B-Cpf1-7b-R | CCAAAGAAGGCGGGAAAGAA | 75 |
| gGRIN2B-Cpf1-13a-F | CAGCTGACCACTGTGATGC | 76 |
| gGRIN2B-Cpf1-13a-R | ATCACTGATAGGTATAAAGTCA | 77 |
| | AAACC | |
| gGRIN2B-Cpf1-16a-F | TTAGCTATGGACAGGAGGGG | 78 |
| gGRIN2B-Cpf1-16a-R | AATATGTTGTGTGCGCCCTC | 79 |
| gGRIN2B-Cpf1-16b-F | TTAGCTATGGACAGGAGGGG | 78 |
| gGRIN2B-Cpf1-16b-R | AATATGTTGTGTGCGCCCTC | 79 |
| gRUNX1-Cpf1-3a-F | GGCAGAGCTGGTGAATGAAC | 80 |
| gRUNX1-Cpf1-3a-R | TGCGACTTCTCTGTTCGTCT | 81 |
| gRUNX1-Cpf1-3b-F | GGCAGAGCTGGTGAATGAAC | 80 |
| gRUNX1-Cpf1-3b-R | TGCGACTTCTCTGTTCGTCT | 81 |
| gRUNX1-Cpf1-6a-F | GCAATAACCTTCACCTCTCGTA | 82 |
| | A | |
| gRUNX1-Cpf1-6a-R | GGGGTCTATAGATTTAGTTGAA | 83 |
| | TTTTC | |
| gRUNX1-Cpf1-10a-F | AGCATTTCAGGATGGGTCTT | 84 |
| gRUNX1-Cpf1-10a-R | TCATTCAAAATAACATTGAAAA | 85 |
| | GAACA | |
| gR26-Cpf1-5c-F | CAAGGCAGACAACCAAGAAA | 86 |
| gR26-Cpf1-5c-R | GGGAAACCCAAAGAAGTGCT | 87 |
| gR26-Cpf1-6d-F | TAGTTATGAGGAGTGAGGTGGA | 88 |
| | CT | |
| gR26-Cpf1-6d-R | AAGCAAATTAACATTAAAAGTC | 89 |
| | AGAAA | |
| gR26-Cpf1-8c-F | GGTTTCCCTGCACTATCCTG | 90 |
| gR26-Cpf1-8c-R | TTGCCTGCAAACCACAATTA | 91 |

3. The Effect of Cpf1 End Retention Asymmetry on NHEJ Repair Depends on c-NHEJ Activity

Since the free PAM-distal end generated after Cpf1 cleaves DNA targets is more likely to bind to KU70/KU80 and undergo efficient and accurate c-NHEJ repair, while the Cpf1-retained PAM-proximal end hinders KU70/KU80 binding and is prone to processing. Then, if the cells lack KU70/KU80 or XRCC4 (a core factor recruited downstream of KU70/KU80), the asymmetry in binding to KU70/KU80 will be disrupted, and the difference in the length of base deletions between the Cpf1-retained end and the free end after Cpf1 cleaves the DNA target will disappear. To verify this possibility, we knocked out KU80 or XRCC4 in mouse embryonic stem cells and analyzed the changes in the difference in the length of base deletions between the Cpf1-retained end and the free end. The details are as follows:

(1) Based on the target sequence at the Rosa26 locus in mouse embryonic stem cells, an sgRNA g5c was designed. The targeting sequence of g5c is 5′-ACAAATCACAAAACCAACATTTG-3′ (SEQ ID NO: 49), with its PAM (5′-TTTC-3′) located on Crick strand. The aforementioned g5c targeting sequence (5′-ACAAATCACAAAACCAACATTTG-3′, SEQ ID NO: 49) was synthesized and ligated into the pU6-LbCpf1-crRNA vector to generate the g5c expression plasmid.

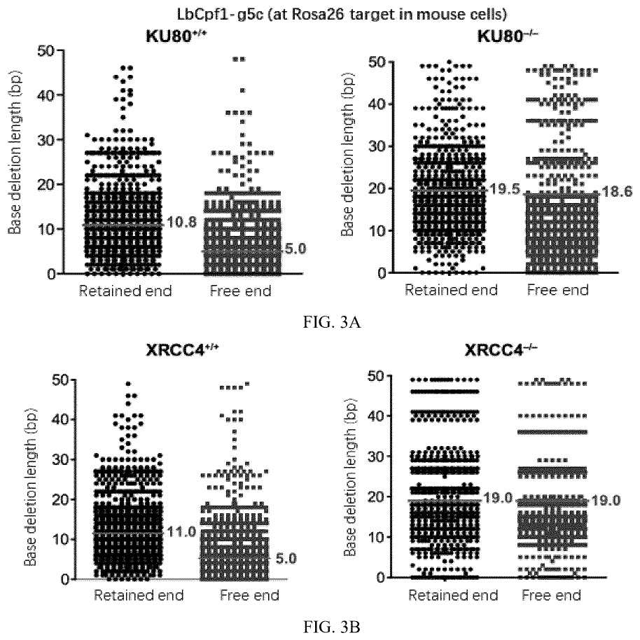

(2) Lipofectamine 2000 liposome transfection method was used to co-transfect the g5c expression plasmid (constructed in Step 1) and Cpf1 expression plasmid (from Addgene) into isogenic KU80^{+/+} and KU80^{-/-} mouse embryonic stem cells (FIG. 3A), as well as isogenic XRCC4^{+/+} and XRCC4^{-/-} mouse embryonic stem cells (FIG. 3B). The transfection system (taking a 24-well plate as an example) is: each well contained 2×10⁵ cells; the total amount of transfected DNA was 0.5 μg, including 0.25 μg of Cpf1 expression plasmid and 0.25 μg of g5c expression plasmid.

(3) 72 hours after transfection, cellular genomic DNA was extracted, and sequences containing the target ligation junction were amplified by PCR. Through appropriate primer design, DNA fragments with PCR products less than 250 bp were obtained. The forward primer F and reverse primer for were R amplifying the LbCpf1-g5c target 5′-CAAGGCAGACAACCAAGAAA-3′ (SEQ ID NO: 86) and 5′-GGGAAACCCAAAGAAGTGCT-3′ (SEQ ID NO: 87), respectively.

(4) After PCR target amplification, the PCR products were subjected to deep sequencing to analyze the base deletion lengths at the PAM-proximal and PAM-distal ends. In KU80^{+/+} cells, the average deletion length at the LbCpf1-free end (i.e., PAM-distal end) was 5.0 bp, while that at the LbCpf1-retained end (i.e., PAM-proximal end) was 10.8 bp. However, after KU80 knockout (i.e., in KU80^{-/-} cells), the average deletion length at the LbCpf1 free end (18.6 bp) was close to that at the LbCpf1-retained end (i.e., PAM-proximal end, 19.5 bp) (FIG. 3A). In XRCC4^{+/+} cells, the average base deletion length at the LbCpf1-free end (i.e., PAM-distal end) was 5.0 bp, while that at the LbCpf1-retained end (i.e., PAM-proximal end) was 11 bp. In XRCC4^{-/-} cells, the average base deletion length at the LbCpf1-free end was close to that at the LbCpf1-retained end (i.e., PAM-proximal end), with both being 19 bp (FIG. 3B). These results indicate that the effect of Cpf1 end retention asymmetry on the base deletion lengths at the two ends of NHEJ repair depends on c-NHEJ activity.

4. Effect of End Retention Asymmetry on c-NHEJ Repair Accuracy after Paired Cpf1 Cleaves DNA Targets

Since Cpf1 end retention asymmetry leads to differences in KU70/KU80 binding and deletion lengths at the two ends, it should also affect the efficiency and accuracy of NHEJ. However, when considering a single Cpf1 target, the DSB generated by cleavage has one retained end and one free end, making it impossible to clearly distinguish the respective effects of the retained end and free end on efficiency and accuracy during ligation. Therefore, we adopted a strategy of paired Cpf1 DNA cleavage target to analyze the respective effects of the Cpf1-retained end and free end on NHEJ repair accuracy. The details are as follows:

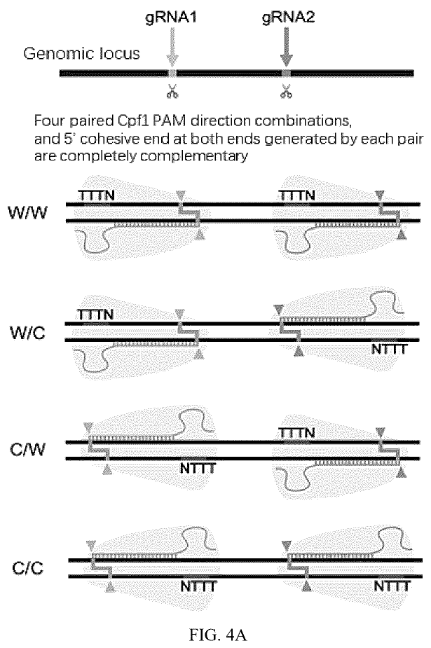

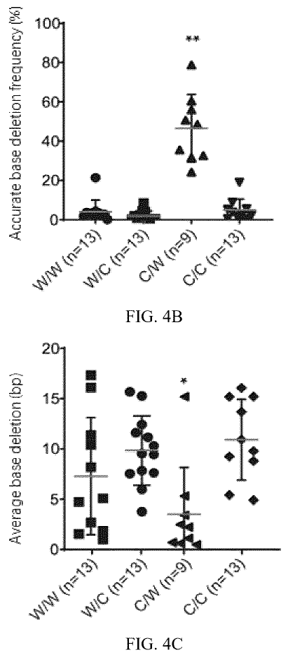

(1) Four Cpf1-sgRNA pairing combinations targeting two adjacent Cpf1 targets were designed at a genomic locus, namely the W/W, W/C, C/C, and C/W combinations (FIG. 4A), where W and C represent the Cpf1 PAM located on Watson strand and Crick strand, respectively. When Cpf1 of any PAM pairing combination cleaves DNA simultaneously, it may delete the intervening sequence between the paired targets. Among them, the C/W PAM combination deletes the intervening sequence containing PAM-proximal ends (i.e., Cpf1-retained ends) at both ends, generating two completely complementary 5′-cohesive Cpf1-free ends to be ligated with each other; the W/C combination deletes the intervening sequence containing PAM-distal ends (i.e., Cpf1-free ends) at both ends, generating two completely complementary 5′-cohesive Cpf1-retained ends to be ligated with each other; the W/W and C/C combinations each delete the intervening sequence containing one PAM-distal end (i.e., Cpf1-free end) and one PAM-proximal end (i.e., Cpf1-retained end) at either end, generating a completely complementary 5′-cohesive Cpf1-retained end and a Cpf1-free end to be ligated with each other (FIG. 4A).

(2) Based on the above design principles, 48 genomic targets targeted by paired LbCpf1-sgRNAs were selected from the genomic sequences of mouse embryonic stem cells or human HEK293 cells, including 13 W/W combinations, 13 W/C combinations, 9 C/W combinations, and 13 C/C combinations. 48 pairs of LbCpf1 gRNAs were designed and synthesized respectively (gRNA sequences are shown in Table 6) and ligated into the pU6-LbCpf1-crRNA vector to form 48 pairs of gRNA expression plasmids.

(3) Lipofectamine 2000 liposome transfection method was used to co-transfect the LbCpf1 expression plasmid and its paired gRNA expression plasmids into mouse embryonic stem cells or HEK293 cells. The transfection system for mouse embryonic stem cells (taking 24-well plate as an example) was as follows: 2×10⁵ cells per well in a 24-well plate, with a total transfected DNA amount of 0.5 μg, including 0.25 μg of LbCpf1 expression plasmid and 0.25 μg of paired sgRNA expression plasmids (0.125 μg for each sgRNA expression plasmid). The transfection system for HEK293 cells (taking 24-well plate as an example) was as follows: 1.4×10⁵ cells per well in a 24-well plate, with a total transfected DNA amount of 0.5 μg, including 0.25 μg of LbCpf1 expression plasmid and 0.25 μg of paired sgRNA expression plasmids (each 0.125 μg).

(4) 27 hours after transfection, cellular genomic DNA was extracted, and the sequences containing the target ligation junctions were amplified by PCR to obtain PCR products. Through appropriate primer design, DNA fragments with PCR products less than 250 bp were obtained. The sequences of PCR primers used for amplifying each paired LbCpf1 target are shown in Table 7.

(5) After PCR amplification of the targets, the PCR products were subjected to deep sequencing. Sequences with deletion of the intervening sequence between the paired LbCpf1 targets were selected, and the NHEJ junction sequences were analyzed to determine the ligation accuracy. We found that the paired C/W PAM combination induced the highest NHEJ product accuracy (close to 50%), which was much higher than that of the other three combinations (all approximately 5%) (FIG. 4B). This is because the C/W combination generates two ligatable completely complementary 5′-cohesive Cpf1-free ends, indicating that the ligation accuracy of the two complementary Cpf1-free ends after paired Cpf1 cleaves DNA targets is significantly higher than that of the complementary ligation involving Cpf1-retained ends. In addition, based on the average length of additional deletions at the ligation ends after deletion of the intervening sequence, we found that the average value of the additional deletion length induced by the C/W combination was less than 5 bp, which was lower than that of the other three combinations (all greater than 6 bp) (FIG. 4C).

TABLE 6
gRNA sequences of 48 pairs of paired LbCpf1 targets

| | Strand | | | gRNA sequence | SEQ ID |
|---|---|---|---|---|---|
| Paired gRNA | with PAM | gRNA name | PAM | (5′-3′) | NO: |
| mRosa26 #2 | C/C | gR26-Cpf1-2a | TTTT | CTGTCCCTGAGCC | 92 |
| | | | | CCCCACCTCC | |
| | | gR26-Cpf1-2c | TTTA | TACCCTTTTCAGG | 93 |
| | | | | AGAGGCCTCC | |
| mRosa26 #4 | C/W | gR26-Cpf1-4a | TTTT | AGTAAGCAGTAAT | 94 |
| | | | | CAATACCATG | |
| | | gR26-Cpf1-4b | TTTC | TCTTGGACTGGCT | 95 |
| | | | | TGACTCATGG | |
| mRosa26 #5 | W/C | gR26-Cpf1-5b | TTTC | ATTCAAGTTTTCC | 96 |
| | | | | CCCATCAAAT | |
| | | gR26-Cpf1-5c | TTTC | ACAAATCACAAAA | 49 |
| | | | | CCAACATTTG | |
| mRosa26 #6 | W/W | gR26-Cpf1-6b | TTTG | TCTTATACTTAAC | 97 |
| | | | | TTTTTTTTTA | |
| | | gR26-Cpf1-6d | TTTA | GAACCAAGGGTCT | 50 |
| | | | | TAGAGTTTTA | |
| mRosa26 #7 | C/C | gR26-Cpf1-7a | TTTC | TAATCTTACCTAT | 98 |
| | | | | TCCTAAAATT | |
| | | gR26-Cpf1-7c | TTTC | TAGAGAAGCCATT | 99 |
| | | | | TCTCAAAATT | |
| mRosa26 #8 | W/C | gR26-Cpf1-8b | TTTC | ATATATCAAGGCA | 100 |
| | | | | AAACATGTTA | |
| | | gR26-Cpf1-8c | TTTC | ACATTAAGTACAA | 51 |
| | | | | ATGTTTAACA | |
| mRosa26 #9 | W/W | gR26-Cpf1-9b | TTTG | GATCTCCTTTTGA | 101 |
| | | | | CAACAATAGC | |
| | | gR26-Cpf1-9d | TTTC | GAGACAGGGTTTC | 102 |
| | | | | TCTGTATAGC | |
| mRosa26 #10 | W/C | gR26-Cpf1-10b | TTTC | AATGAGTGTCAGA | 103 |
| | | | | TTGTTTTGAA | |
| | | gR26-Cpf1-10c | TTTC | TAAGCTATTACAC | 104 |
| | | | | TTTCCTTCAA | |
| mRosa26 #11 | W/W | gR26-Cpf1-11b | TTTC | AAAATTTTAGGAA | 105 |
| | | | | TAGGTAAGAT | |
| | | gR26-Cpf1-11d | TTTG | AGAAATGGCTTCT | 106 |
| | | | | CTAGAAAGAT | |
| mRosa26 #12 | C/C | gR26-Cpf1-12a | TTTC | ACATAACTAAAACC | 107 |
| | | | | AAGCACAGTG | |
| | | gR26-Cpf1-12c | TTTG | GTTCACACCACAA | 108 |
| | | | | ATGAACAGTG | |
| mRosa26 #13 | C/C | gR26-Cpf1-13a | TTTG | TAAGCTTCATCCA | 109 |
| | | | | TTTGTAACAT | |
| | | gR26-Cpf1-13c | TTTC | ACCATTAGGGCAA | 110 |
| | | | | ATGGCAACAT | |
| mCola1 1# | W/C | gCola1-Cpf1-1a | TTTA | ATCCCAGCACTAA | 111 |
| | | | | GGAAGCAGAG | |
| | | gCola1-Cpf1-1b | TTTG | GTTTTCAAGACAG | 112 |
| | | | | GGTTTCTCTG | |
| mCola1 2# | C/C | gCola1-Cpf1-2a | TTTC | TCACCAGCAGGAC | 113 |
| | | | | CGGGGGGACC | |
| | | gCola1-Cpf1-2b | TTTG | CCCCCTTCTTTGCC | 114 |
| | | | | AACGGGACC | |
| HBB #2 | C/W | gHBB-Cpf1-2a | TTTA | CTATTATACTTAA | 115 |
| | | | | TGCCTTAACA | |
| | | gHBB-Cpf1-2b | TTTC | CCATTCTAAACTG | 116 |
| | | | | TACCCTGTTA | |
| HBB #3 | C/C | gHBB-Cpf1-3a | TTTC | ATATTGCTAATAG | 117 |
| | | | | CAGCTACAAT | |
| | | gHBB-Cpf1-3c | TTTC | TTTCAGGGCAATA | 118 |
| | | | | ATGATACAAT | |
| HBD #1 | C/C | gHBD-Cpf1-1a | TTTC | TCTCCCAACCCCC | 119 |
| | | | | TCCCTTCATT | |
| | | gHBD-Cpf1-1c | TTTG | TCATTTTACTATAT | 120 |
| | | | | TTTATCATT | |
| HBD #2 | C/C | gHBD-Cpf1-2a | TTTC | CTTCCTCACAATC | 121 |
| | | | | TTGCTATTTT | |
| | | gHBD-Cpf1-2c | TTTA | TCATTTAATGCTT | 122 |
| | | | | CTAAAATTTT | |
| AAVS1 #1 | C/C | gAAVS1-Cpf1- | TTTG | ACCCACTCCCCAT | 36 |
| | | 1a | | TTCAACCAAA | |
| | | gAAVS1-Cpf1- | TTTC | TGTAAATCTGAAG | 37 |
| | | 1c | | CTTTCCCAAA | |
| AAVS1 #2 | C/W | gAAVS1-Cpf1- | TTTG | ACTATTCTGGGTA | 123 |
| | | 2a | | CCTCACAGAC | |
| | | gAAVS1-Cpf1- | TTTC | ACCACGTTGCCCA | 124 |
| | | 2b | | AGCTAGTCTG | |
| AAVS1 #3 | C/C | gAAVS1-Cpf1- | TTTG | TTTTTTGTTTTGAG | 125 |
| | | 3a | | ACACAGTTT | |
| | | gAAVS1-Cpf1- | TTTG | TATTTTTAGTAGA | 126 |
| | | 3c | | GACAGAGTTT | |
| AAVS1 #4 | C/W | gAAVS1-Cpf1- | TTTA | AATGCACCTCTCA | 127 |
| | | 4a | | TAGTAACTGA | |
| | | gAAVS1-Cpf1- | TTTG | TTTCTTCTTATAGT | 128 |
| | | 4b | | TCTGTCAGT | |
| EMX1 #1 | C/C | gEMX1-Cpf1- | TTTC | TCTCAGTCTTTTG | 129 |
| | | 1a | | ACTTGTCTTT | |
| | | gEMX1-Cpf1- | TTTC | CTATTCAGCCTGA | 130 |
| | | 1c | | CTTCATCTTT | |
| EMX1 #2 | W/W | gEMX1-Cpf1- | TTTC | ACCTTCCGCTCCC | 131 |
| | | 2b | | CTTTCCTCCT | |
| | | gEMX1-Cpf1- | TTTC | TTCGGCGGACCTT | 132 |
| | | 2d | | ACCCTCTCCT | |
| EMX1 #3 | W/C | gEMX1-Cpf1- | TTTC | CCTCCTGGCAGTG | 133 |
| | | 3b | | TTTTAAAATT | |
| | | gEMX1-Cpf1- | TTTC | TTACAGTTCTGCA | 134 |
| | | 3c | | ATTAAAATTT | |
| EMX1 #4 | W/W | gEMX1-Cpf1- | TTTG | CTGATTAGAATTC | 38 |
| | | 4b | | TACCCCTCTT | |
| | | gEMX1-Cpf1- | TTTC | CAGGTCTGGCCCA | 135 |
| | | 4d | | GGTACCTCTT | |
| FANCF #1 | W/W | gFANCF-Cpf1- | TTTC | TGCAAATTCTTAC | 136 |
| | | 1b | | TTTGAAAATG | |
| | | gFANCF-Cpf1- | TTTA | AAGCATTGACGCA | 137 |
| | | 1d | | CAGACAAATG | |
| FANCF #2 | C/C | gFANCF-Cpf1- | TTTG | TAAACTATATATT | 138 |
| | | 2a | | CTATATTCAA | |
| | | gFANCF-Cpf1- | TTTA | TAACTGTTTAATA | 139 |
| | | 2c | | AACTATTCAA | |
| hVEGFA 1# | W/W | gVEGFA-Cpf1- | TTTA | AAAAGTCTTTTGG | 140 |
| | | 1a | | TGTTACCTGG | |
| | | gVEGFA-Cpf1- | TTTG | TTTGGGAAGCTGG | 141 |
| | | 1b | | ATGAGCCTGG | |
| hRUNX1 1# | C/W | gRUNX1-Cpf1- | TTTA | AGTTTATTTCACA | 142 |
| | | 1a | | GGACAAGAGT | |
| | | gRUNX1-Cpf1- | TTTG | GTAATCAAAGAGC | 143 |
| | | 1b | | CCTTAACTCT | |
| hRUNX1 2# | W/C | gRUNX1-Cpf1- | TTTA | TGTATTATCGATG | 144 |
| | | 2a | | GCTGCTTTCT | |
| | | gRUNX1-Cpf1- | TTTA | GATACTCTAAAGT | 145 |
| | | 2b | | AGAGTAGAAA | |
| hRUNX1 3# | C/W | gRUNX1-Cpf1- | TTTC | CTCAGTTCCTACC | 45 |
| | | 3a | | AGGACAGCTG | |
| | | gRUNX1-Cpf1- | TTTG | GAGAAGAGGGAG | 46 |
| | | 3b | | AGAAAACAGCT | |
| hRUNX1 4# | W/W | gRUNX1-Cpf1- | TTTC | TTCCTGTCTGCAG | 146 |
| | | 4a | | AGCTGTGAAA | |
| | | gRUNX1-Cpf1- | TTTC | TTTGCTGCAAGGC | 147 |
| | | 4b | | AAGGGTGAAA | |
| hRUNX1 5# | C/W | gRUNX1-Cpf1- | TTTC | CTTTCTCTTTACCA | 148 |
| | | 5a | | TGCTGTGAC | |
| | | gRUNX1-Cpf1- | TTTA | TCAAAACTACTCA | 149 |
| | | 5b | | ACTCTGTCAC | |
| hRUNX1 6# | C/C | gRUNX1-Cpf1- | TTTC | ACCCTTGCCTTGC | 47 |
| | | 6a | | AGCAAAGAAA | |
| | | gRUNX1-Cpf1- | TTTC | ACAGCTCTGCAGA | 150 |
| | | 6b | | CAGGAAGAAA | |
| hRUNX1 7# | W/W | gRUNX1-Cpf1- | TTTA | CTTGTACATATTTT | 151 |
| | | 7a | | GTTCTTTTC | |
| | | gRUNX1-Cpf1- | TTTA | TTTCAGGATTCTTT | 152 |
| | | 7b | | AGAGTTTTC | |
| hRUNX1 8# | W/W | gRUNX1-Cpf1- | TTTA | TTTCAGGATTCTTT | 152 |
| | | 8a | | AGAGTTTTC | |
| | | gRUNX1-Cpf1- | TTTG | GGTGTCTTTTATA | 153 |
| | | 8b | | TGTTGTTTTC | |
| hRUNX1 9# | W/C | gRUNX1-Cpf1- | TTTG | ATAAATATTTATC | 154 |
| | | 9a | | TTGAATGCCT | |
| | | gRUNX1-Cpf1- | TTTC | CAGCATCTAAAAC | 155 |
| | | 9b | | ATGTCAGGCA | |
| hRUNX1 10# | W/W | gRUNX1-Cpf1- | TTTG | ATGGGGGATTACA | 48 |
| | | 10a | | CTGAATCTTT | |
| | | gRUNX1-Cpf1- | TTTG | TGGTTTTCAGTAT | 156 |
| | | 10b | | ATGAGTCTTT | |
| hGRIN2B 1# | C/C | gGRIN2B-Cpf1- | TTTA | ATTGTCTTAATCT | 157 |
| | | 1a | | AGGAAGCTCC | |
| | | gGRIN2B-Cpf1- | TTTA | CGCTCCCCATCAA | 158 |
| | | 1b | | GCTGGGCTCC | |
| hGRIN2B 2# | W/C | gGRIN2B-Cpf1- | TTTC | AAATATCTCTATC | 159 |
| | | 2a | | AGCTATTAAC | |
| | | gGRIN2B-Cpf1- | TTTA | AAATATGCATGTG | 160 |
| | | 2b | | GTAAAGTTAA | |
| hGRIN2B 3# | W/C | gGRIN2B-Cpf1- | TTTA | CAGATTGTGTTGC | 39 |
| | | 3a | | CATTATTAGT | |
| | | gGRIN2B-Cpf1- | TTTG | TGATAAAGTGAAA | 40 |
| | | 3b | | AGAGCACTAA | |
| hGRIN2B 4# | C/W | gGRIN2B-Cpf1- | TTTC | CAAAGGTAACTAC | 161 |
| | | 4a | | AACAATCTCT | |
| | | gGRIN2B-Cpf1- | TTTA | CCAATGGAGATAC | 162 |
| | | 4b | | TGCCAAGAGA | |
| hGRIN2B 5# | W/C | gGRIN2B-Cpf1- | TTTC | CATGTATGTTCTC | 163 |
| | | 5a | | ACTTAATCCT | |
| | | gGRIN2B-Cpf1- | TTTA | TTCCTTATTTGGA | 164 |
| | | 5b | | AAATAAGGAT | |
| hGRIN2B 6# | C/W | gGRIN2B-Cpf1- | TTTC | TGCCTTTGATCTC | 165 |
| | | 6a | | AGAGGGTCTG | |
| | | gGRIN2B-Cpf1- | TTTG | CAAGTTATTAATA | 166 |
| | | 6b | | TACGCGTCTG | |
| hGRIN2B 7# | C/W | gGRIN2B-Cpf1- | TTTA | AGTGGAGAGTTGG | 167 |
| | | 7a | | TATATTTATA | |
| | | gGRIN2B-Cpf1- | TTTA | TAAGAAAAGCATT | 41 |
| | | 7b | | TTAAATATAA | |
| hGRIN2B 8# | W/W | gGRIN2B-Cpf1- | TTTA | TCCTTGTTTCCCTG | 168 |
| | | 8a | | TACTTAATT | |
| | | gGRIN2B-Cpf1- | TTTC | TCTCATTATTGGA | 169 |
| | | 8b | | GAATATAATT | |
| hGRIN2B 9# | C/W | gGRIN2B-Cpf1- | TTTG | TTCCTTTTTATTGA | 170 |
| | | 9a | | GTATTGAAT | |
| | | gGRIN2B-Cpf1- | TTTG | CTAAGCAAAAAA | 171 |
| | | 9b | | AGCTAGATTCA | |
| hGRIN2B 10# | W/W | gGRIN2B-Cpf1- | TTTA | AATCTTGAACACA | 172 |
| | | 10a | | CATGCTATTT | |
| | | gGRIN2B-Cpf1- | TTTG | ATTTGGCTTGCCA | 173 |
| | | 10b | | AGGTCTATTT | |
| hGRIN2B 11# | W/C | gGRIN2B-Cpf1- | TTTA | TTCTCTGCCTACTC | 174 |
| | | 11a | | TCTCTCTTT | |
| | | gGRIN2B-Cpf1- | TTTA | CCCTGGTCACTGC | 175 |
| | | 11b | | TGTCCTCTTT | |
| hGRIN2B 12# | C/W | gGRIN2B-Cpf1- | TTTC | CCAGAAAGGGGT | 176 |
| | | 12a | | GTGAATCTCTA | |
| | | gGRIN2B-Cpf1- | TTTA | ACCTCCAGTCTTC | 177 |
| | | 12b | | TGAGTTAGAG | |
| hGRIN2B 13# | W/C | gGRIN2B-Cpf1- | TTTC | TAGCATATAGTAT | 42 |
| | | 13a | | AATAAAAAAG | |
| | | gGRIN2B-Cpf1- | TTTC | AAAATATGCAACA | 178 |
| | | 13b | | GTGTTCTTTT | |
| hGRIN2B 14# | W/C | gGRIN2B-Cpf1- | TTTA | TTGCTTCTTGGAA | 179 |
| | | 14a | | TCTGATCTTG | |
| | | gGRIN2B-Cpf1- | TTTC | ACTCAGTCAAAAA | 180 |
| | | 14b | | TTCCACAAGA | |
| hGRIN2B 15# | W/W | gGRIN2B-Cpf1- | TTTA | TTGCTGCTAGATA | 181 |
| | | 15a | | TTCCTTCACA | |
| | | gGRIN2B-Cpf1- | TTTC | TCTCTATTTACTTC | 182 |
| | | 15b | | CTCGTCACA | |
| hGRIN2B 16# | W/C | gGRIN2B-Cpf1- | TTTC | ATCAGGCAAGTGT | 43 |
| | | 16a | | GGGTCCATTT | |
| | | gGRIN2B-Cpf1- | TTTG | CAAGAATCTATGT | 44 |
| | | 16b | | CTCTAAAATG | |
| hGRIN2B 17# | C/W | gGRIN2B-Cpf1- | TTTG | TCTAGGTAAATAT | 183 |
| | | 17a | | TTTAGTTGGC | |
| | | gGRIN2B-Cpf1- | TTTC | TTATAAAATTTAA | 184 |
| | | 17b | | TCTTAGCCAA | |

TABLE 7
[]Primer sequences of 48 pairs of paired LbCpfl targets

| | Primer | | SEQ ID |
|---|---|---|---|
| Paired gRNA | direction | primer sequence (5′-3′) | NO: |
| mRosa26 #2 | F | GGAGAGGCGTTCAGGAAGAT | 185 |
| | R | TCTAGTCGACCCCACTACCT | 186 |
| mRosa26 #4 | F | TCTTACATATTGCCAGGCTGAT | 187 |
| | R | GTCCTGAAGAAGCTTGGCAA | 188 |
| mRosa26 #5 | F | CAAGGCAGACAACCAAGAAA | 86 |
| | R | GGGAAACCCAAAGAAGTGCT | 87 |
| mRosa26 #6 | F | TAGTTATGAGGAGTGAGGTGGACT | 88 |
| | R | AAGCAAATTAACATTAAAAGTCAGAAA | 89 |
| mRosa26 #7 | F | TGTTCTCACTGAGCTACATCCTG | 189 |
| | R | CTGTGACCCACGTAAAGCAA | 190 |
| mRosa26 #8 | F | GGTTTCCCTGCACTATCCTG | 90 |
| | R | TTGCCTGCAAACCACAATTA | 91 |
| mRosa26 #9 | F | AGCCCTTGTTCTTTATCACCCT | 191 |
| | R | AATATCCAACTTAGCCAGGCGT | 192 |
| mRosa26 #10 | F | TGTGTTGGTGCGAGCAAT | 193 |
| | R | TCTGCCAGATATTCAGCAATG | 194 |
| mRosa26 #11 | F | TGTTCTCACTGAGCTACATCCTG | 189 |
| | R | CTGTGACCCACGTAAAGCAA | 190 |
| mRosa26 #12 | F | GGCTTGACTTGTCACTGTGCT | 195 |
| | R | GCCCAATTCCAACTGTGAAG | 196 |
| mRosa26 #13 | F | ACTCAGTGGTTCTTTTGAGCA | 197 |
| | R | CCCCACTTTTTCTTTCACCA | 198 |
| mCola1 1# | F | AGACAAGGAGAGCAAATGTGA | 199 |
| | R | ACCTTGTTTGCCAGGTTCAC | 200 |
| mCola1 2# | F | GGCTTGCCACTATGATGCTT | 201 |
| | R | ACACATACACAACTCTGGAACTC | 202 |
| HBB #2 | F | AAGTTACTTAATGTATCTCAGAGATA | 203 |
| | R | ATGGGACGCTTGATGTTTTC | 204 |
| HBB #3 | F | GGCCTAGCTTGGACTCAGAA | 205 |
| | R | TTTTTGTTTATCTTATTTCTAATACTTTCC | 206 |
| HBD #1 | F | TGCAGAATTAGCAGGTGAGAG | 207 |
| | R | CCAGGAGATGCTTCACTTTTCT | 208 |
| HBD #2 | F | TGCAGAATTAGCAGGTGAGAG | 207 |
| | R | CCAGGAGATGCTTCACTTTTCT | 208 |
| AAVS1 #1 | F | GCCAGGCTGAAAGGATAGG | 68 |
| | R | AGAAAAAGCACTGGCGAATC | 69 |
| AAVS1 #2 | F | TGAAAAGTGAAAATAAGCCAGTCA | 209 |
| | R | CTGCAATCCCAGCACTTTAG | 210 |
| AAVS1 #3 | F | TGAGGTCGGGAGTTTGAGG | 211 |
| | R | AGCATAATGTCCTCAAGATACATCTAC | 212 |
| AAVS1 #4 | F | GGTCAACCTTGTAATCATGCTGT | 213 |
| | R | GTGGAGGTTGCAGTGAGCTA | 214 |
| EMX1 #1 | F | TTTGCAAAAGCCATTTTCCT | 215 |
| | R | AGAATTTGGACCAGCCACAC | 216 |
| EMX1 #2 | F | GCGGTCTCCCAGCTACCTC | 217 |
| | R | AGATTAAGTGGGGCAGCAGA | 218 |
| EMX1 #3 | F | TGCAGGGAAAAAGCTTACAAA | 219 |
| | R | TGTTAATCTGTGGGTGGTAGGA | 220 |
| EMX1 #4 | F | CCCTCTTGCAAAAGTGTCCA | 70 |
| | R | TTTACGTCTGCCTCCTCACC | 71 |
| FANCF #1 | F | CCAGTTAACCAGCCTTAGTATGC | 221 |
| | R | GAGGCAGAGGTTGCACTGA | 222 |
| FANCF #2 | F | AGCTCTTCGTAGTGGTGCATTTA | 223 |
| | R | TCCAATCACTTCCTCTATCCAGA | 224 |
| hVEGFA 1# | F | CAGGAGGGGACAGATGGATG | 225 |
| | R | CATCCCCTCCCCTTCTTTCA | 226 |
| hRUNX1 1# | F | GGTGCCTGACGAATAAGCTG | 227 |
| | R | AAATACTGATGATCCCCACTAGG | 228 |
| hRUNX1 2# | F | GAAGTCCGTGGGCCAAATC | 229 |
| | R | AGCATGGTAAAGAGAAAGGAAAGT | 230 |
| hRUNX1 3# | F | GGCAGAGCTGGTGAATGAAC | 80 |
| | R | TGCGACTTCTCTGTTCGTCT | 81 |
| hRUNX1 4# | F | AGAGTTGCTGTTATTCTGGTAG | 231 |
| | R | AGCCATTGGTACCTGTAAGTAAA | 232 |
| hRUNX1 5# | F | GCCCGTTCATTTATGTATTATCGA | 233 |
| | R | TGAGCATGACTTTGGACAATAATTT | 234 |
| hRUNX1 6# | F | GCAATAACCTTCACCTCTCGTAA | 82 |
| | R | GGGGTCTATAGATTTAGTTGAATTTTC | 83 |
| hRUNX1 7# | F | TGAGTCTTTTGCCTCCTTGTTTA | 235 |
| | R | TGGAAAGTAAGGAGTCAAATTATCTCT | 236 |
| hRUNX1 8# | F | TGTAGAAACACAACTGCTCTTTG | 237 |
| | R | AGATCAGGAACAAGACAAGGA | 238 |
| hRUNX1 9# | F | TGTGAGGCGAAAATCCCATT | 239 |
| | R | TGTGCCCGGCCTACTTATTT | 240 |
| hRUNX1 10# | F | AGCATTTCAGGATGGGTCTT | 84 |
| | R | TCATTCAAAATAACATTGAAAAGAACA | 85 |
| hGRIN2B 1# | F | CTGAGAAGGCGGTGGAGG | 241 |
| | R | CTGAGTGAGGCAGTGAGGAA | 242 |
| hGRIN2B 2# | F | ACAGGGGTCATCATTGTTAAAGA | 243 |
| | R | CCAGTGCCTCTCTCCTCAAT | 244 |
| hGRIN2B 3# | F | CTGGCGTTTGGACTCAGTTC | 72 |
| | R | CGCTGGTCTCAAGTCTCTTG | 73 |
| hGRIN2B 4# | F | GGGGAGATATATTGTAGCTTCTC | 245 |
| | R | TGTGCAGTAATGACCAGGTC | 246 |
| hGRIN2B 5# | F | GGCAAATGAGAAACCTAGGCC | 247 |
| | R | AAGTGGGAGCTCTGTAGTCA | 248 |
| hGRIN2B 6# | F | GGCAAATGAGAAACCTAGGCC | 247 |
| | R | AAGTGGGAGCTCTGTAGTCA | 248 |
| hGRIN2B 7# | F | TGGATCATGTAGTTGCAGGGT | 74 |
| | R | CCAAAGAAGGCGGGAAAGAA | 75 |
| hGRIN2B 8# | F | TCATCTTGTGTTGTTTCCCTTCA | 249 |
| | R | ACTTCTGAAGACCTGGCTAAAG | 250 |
| hGRIN2B 9# | F | GCCCTTTTGCAGTCAGTTTT | 251 |
| | R | GAAATGATCCACTTGTCCATAAAT | 252 |
| hGRIN2B 10# | F | CACATTGAACTTCTGATTGATATTATC | 253 |
| | R | CAGAGGGAGCTGCTTATAAAA | 254 |
| hGRIN2B 11# | F | CACATTGAACTTCTGATTGATATTATC | 253 |
| | R | CAGAGGGAGCTGCTTATAAAA | 254 |
| hGRIN2B 12# | F | CAGAAGGTCTCTAAGAACCAAAC | 255 |
| | R | ATGTATTTAATACCTATCTTGTTCTCA | 256 |
| hGRIN2B 13# | F | CAGCTGACCACTGTGATGC | 76 |
| | R | ATCACTGATAGGTATAAAGTCAAAACC | 77 |
| hGRIN2B 14# | F | CTCTCTTAGGGCCAATCTCAGT | 257 |
| | R | AGAGCTTTGAGGCCAGGAAA | 258 |
| hGRIN2B 15# | F | AGAAACAAGAACTTTTACAATGATGCT | 259 |
| | R | CTGACGGCTGACACTTCAC | 260 |
| hGRIN2B 16# | F | TTAGCTATGGACAGGAGGGG | 78 |
| | R | AATATGTTGTGTGCGCCCTC | 79 |
| hGRIN2B 17# | F | TTAATAGTAATAAGATCTGAGCCCCA | 261 |
| | R | GTTTCTAAAGACAGGTCATCAGAGAT | 262 |

### 5. Effect of Cpf1 End Retention Asymmetry on Translocation Ligation Efficiency

To test the respective effects of the Cpf1-retained end and free end on NHEJ repair efficiency, we compared the translocation efficiency between each end of the Cpf1-cleaved target and the same end of the SpCas9-cleaved target. The details are as follows:

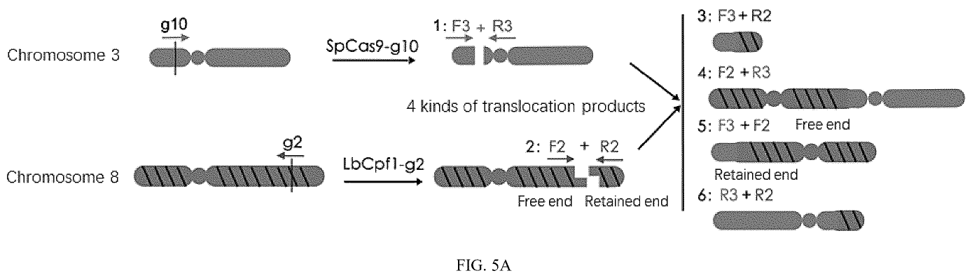

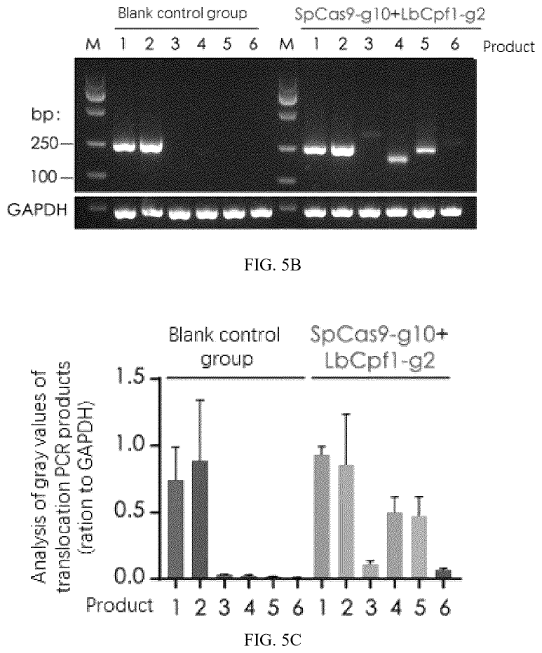

(1) Design sgRNA g10 of SpCas9 targeting chromosome 3. The spacer sequence of g10 is 5′-GATCGAATCTTCTAGCCCTT-3′ (SEQ ID NO: 266), and its PAM (5′-TGG-3′) is located on Watson strand (FIG. 5A). Design sgRNA g2 of LbCpf1 targeting chromosome 8. The spacer sequence of g2 is 5′-GGCAAATAGGAATGGCAAGAGGG-3′ (SEQ ID NO: 267), and its PAM (5′-TTTG-3′) is located on Crick strand (FIG. 5A). Synthesize the aforementioned sgRNA spacer sequences, and ligate the g10 and g2 spacer sequences into the px330-U6-Chimeric vector and pU6-LbCpf1-crRNA vector, respectively, to form the expression plasmids of g10 and g2.

(2) Lipofectamine 2000 liposome transfection method was used to co-transfect LbCpf1, SpCas9, and their respective gRNA expression plasmids into HEK293T cells. The transfection system (taking a 24-well plate as an example) is as follows: 2×10⁵ cells per well in a 24-well plate, with a total transfected DNA amount of 0.5 μg, including 0.2 μg of LbCpf1 expression plasmid, 0.2 μg of SpCas9 expression plasmid, and 0.1 μg each of sgRNA g2 and g10 expression plasmids. A blank control group was set up simultaneously, where the gRNA plasmids in the transfection system were replaced with the empty vector U6, while other components remained unchanged.

(3) 72 hours after transfection, genomic DNA is extracted, and the sequences containing the translocation junctions between the LbCpf1 target and SpCas9 target are amplified by PCR. Through appropriate primer design, the size of the translocation PCR products is controlled to be less than 250 bp. Design primers F2 and R2 at the LbCpf1 site: 5′-CAAACAGCTGACCTTGTGCTT-3′ (SEQ ID NO: 268) and 5′-GAGTGGCTCCAACTTCCTTGTA-3′ (SEQ ID NO: 269), respectively; design primers F3 and R3 at the SpCas9 site: 5′-GAGATCGAGAGGTACGGCTG-3′ (SEQ ID NO: 270) and 5′-CTCCAACTGCCCTTCTGTCC-3′ (SEQ ID NO: 271), respectively. A pair of primers F and R for the internal reference GAPDH are 5′-GTCATCCCTGAGCTGAACG-3′ (SEQ ID NO: 272) and 5′-GTCAAAGGTGGAGGAGTGG-3′ (SEQ ID NO: 273), respectively. SpCas9-g10 and LbCpf1-g2 cleave targets on chromosome 3 and chromosome 8, respectively, and the broken ends can undergo two types of direct ligation (i.e., 1 and 2) and four different combinations of translocation (3, 4, 5, and 6) (FIG. 5A). These products can be detected by PCR: paired primers F3/R3 detect direct ligation product 1, F2/R2 detect direct ligation product 2, paired primers F3/R2 detect translocation product 3, paired primers F2/R3 detect translocation product 4, paired primers F3/F2 detect translocation product 5, and paired primers R3/R2 detect translocation product 6. Meanwhile, PCR amplification of the GAPDH gene fragment is performed as an internal reference.

(4) The PCR amplification products of translocations can be detected by agarose gel electrophoresis. The agarose gel electrophoresis results show that the PCR results of the blank control group can only amplify the two direct ligation products 1 and 2 without any translocation products, while the experimental group can detect the PCR products of translocations 3, 4, 5, and 6 in addition to the two direct ligation products 1 and 2. Moreover, the band brightness of translocation products 4 and 5 is higher than that of translocation products 3 and 6 (FIG. 5B). Gray value analysis is performed on the translocation PCR product bands in the agarose gel electrophoresis to calculate the quantitative results of the PCR products, which are represented by the ratio of the gray value of the PCR product to that of GAPDH. The PCR quantitative results show that the yields of products 4 and 5 are 5 times higher than those of products 3 and 6 (FIG. 5C). This indicates that the translocation ligation efficiency of the LbCpf1-free end is much higher than that of the LbCpf1-retained end.

Therefore, based on the results in FIGS. 1A-5C, the present invention uses single or paired Cpf1 to generate free 5′-cohesive target DNA ends and free 5′-cohesive donor DNA ends, and achieves more efficient and more accurate gene knock-in based on NHEJ by designing complementary sequences for the 5′-cohesive ends.

### Example 2: N-Terminal Accurate Gene Knock-In Using Cpf1-Free Ends

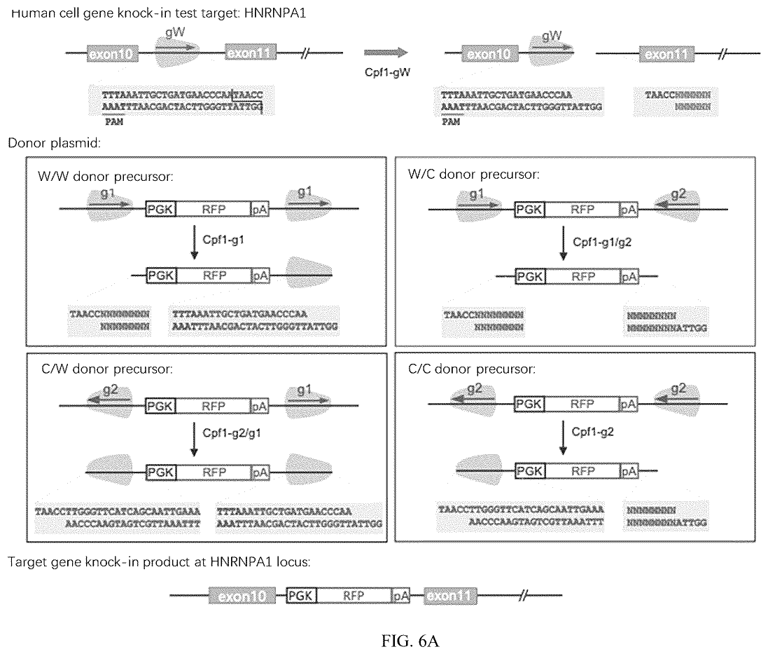

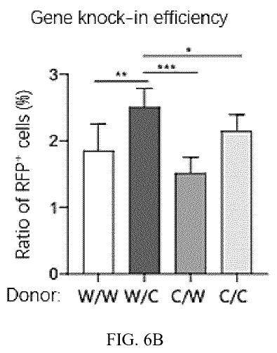

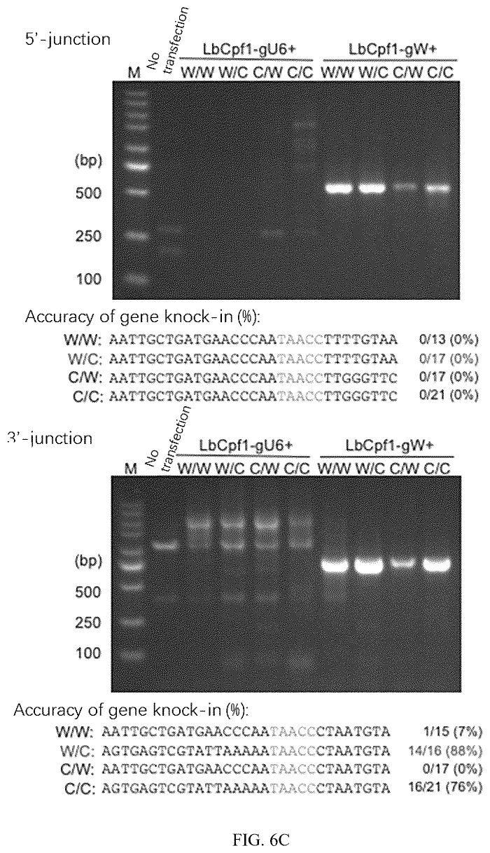

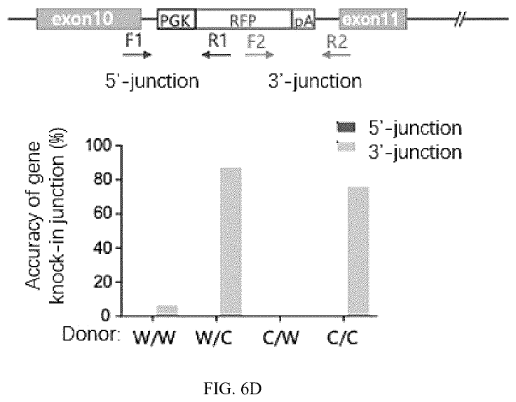

Knock in the red fluorescent protein RFP gene into intron 10 (NCBI Gene ID: 3178) of the HNRNPA1 gene in human HEK293T cells, ensuring precise connection between the N-terminus of the recipient gene insertion site and the RFP gene (FIG. 6A). The specific steps are as follows:

(1) Design and construct the gRNA (i.e., gW) for the Cpf1 target in intron 10 of the HNRNPA1 gene: To achieve precise connection between the N-terminus of the recipient gene insertion site and the RFP gene, design gW for the Cpf1 target in intron 10 of HNRNPA1. Its spacer sequence is 5′-AATTGCTGATGAACCCAATAACC-3′ (SEQ ID NO: 274), and the PAM is 5′-TTTA-3′ located on Watson strand. Cleavage at the Cpf1 target should generate an upstream end that is the Cpf1-retained PAM-proximal end with a 5′-overhang having the base composition of 3′-ATTGG-5′, and a downstream end that is the Cpf1-free PAM-distal end with a 5′-overhang having the base composition of 3′-CCAAT-5′ (FIG. 6A). Synthesize the aforementioned gW sgRNA spacer sequence and ligate it into the pU6-LbCpf1-crRNA vector to form a plasmid expressing sgRNA.

(2) Design donor plasmids carrying Cpf1 targets with four different PAM direction combinations at both ends. W represents Watson strand; C represents Crick strand. The donor plasmid is composed of the HNRNPA1 upstream recognition sequence (PAM on the Watson or Crick strand)-PGK-RFP-pA expression cassette-HNRNPA1 downstream recognition sequence (PAM on the Watson or Crick strand). The PGK-RFP-pA expression cassette is shown in SEQ ID NO: 2: 1-528 bp are the PGK promoter, 529-1308 bp are the RFP coding region, and 1309-1567 bp are the PolyA tail. The HNRNPA1 upstream and downstream recognition sequences can be either g1 in the W direction (5′-AATTGCTGATGAACCCAATAACC-3′ (SEQ ID NO: 274), PAM: 5′-TTTA-3′) or g2 in the C direction (5′-AATTGCTGATGAACCCAAGGTTA-3′ (SEQ ID NO: 275), PAM: 5′-TTTA-3′). Therefore, the combinations of HNRNPA1 upstream and downstream recognition sequences include four types: W/W, W/C, C/W, and C/C. Cleavage of the donor precursor targets by Cpf1 generates a donor with two 5′-cohesive ends: the upstream end can be either the Cpf1-retained PAM-proximal end or the Cpf1-free PAM-distal end, with the 5′-overhang having a base composition of 3′-CCAAT-5′; the downstream end can be either the Cpf1-retained PAM-proximal end or the Cpf1-free PAM-distal end, with the 5′-overhang having a base composition of 3′-ATTGG-5′. Gene-synthesize the aforementioned HNRNPA1 upstream and downstream recognition sequences, and seamlessly ligate them with the 1567 bp red fluorescent protein RFP expression cassette (containing the PGK promoter and PolyA tail) obtained by PCR amplification using the Seamless Cloning Kit (Abclonal, Cat. No. RK21020) to obtain four combinations of donor plasmids (W/W, W/C, C/W, and C/C) (FIG. 6A). Specifically, the PGK-RFP-pA expression cassette is flanked by 27 bp Cpf1-gRNA target sequences containing PAM, forming the four PAM combinations (W/W, W/C, C/W, and C/C).

(3) Construct gRNA g1 and/or gRNA g2 for the Cpf1 targets at both ends of the donor precursor (FIG. 6A). The spacer sequence of gRNA g1 is 5′-AATTGCTGATGAACCCAATAACC-3′ (SEQ ID NO: 274) with a PAM of 5′-TTTA-3′; the spacer sequence of gRNA g2 is 5′-AATTGCTGATGAACCCAAGGTTA-3′ (SEQ ID NO: 275) with a PAM of 5′-TTTA-3′. Design and synthesize the aforementioned sgRNA sequences, then ligate them into the pU6-LbCpf1-crRNA vector to form plasmids expressing g1 and g2 (FIG. 6A).

(4) Detection of gene knock-in efficiency and accuracy. Using the Lipofectamine 2000 liposome transfection method, transfect the four types of donor plasmids from step (2) into HEK293T cells, respectively, together with the gW plasmid targeting the genomic HNRNPA1 locus from step (1), the g1/g2 plasmids targeting the donor precursor loci and the Cpf1 expression plasmid from step (3). Transfection system (taking a 24-well plate as an example): each well contains 1.4×10⁵ cells, with a total transfected DNA amount of 0.8 μg, including 0.4 μg of donor plasmid, 0.2 μg of Cpf1 expression plasmid, and 0.05 μg of each sgRNA (1 from step (1); 2 from step (3), which may be identical). Meanwhile, set up a sgRNA blank control group (U6), where all three sgRNAs are replaced with empty U6 plasmids without sgRNA. To eliminate systematic errors and improve the reliability and accuracy of the experiment, transfection efficiency correction is required. Therefore, the gene knock-in efficiency is corrected using the transfection efficiency of the green fluorescent protein (GFP) gene, i.e., the frequency of RFP cells measured by flow cytometry is multiplied by 100 and divided by the frequency of GFP cells. Results from three independent experiments show that flow cytometry detection 23 days after transfection reveals almost no RFP cells in the U6 control group, while the frequencies of RFP cells generated by the four donors are 1.7% (W/W), 2.4% (W/C), 1.5% (C/W), and 1.9% (C/C), respectively (FIG. 6B). Statistical analysis indicates that the donor with the W/C combination exhibits the highest gene knock-in efficiency.

Collect cells from the U6 control group and the four experimental groups with different donors on day 3, then extract genomic DNA. PCR amplify the 5′ and 3′ junctions of the HNRNPA1 genomic target gene knock-in. The paired primer sequences for detecting the 5′ junction are F1 and R1, 5′-GCAAAACCACGAAACCAA-3′ (SEQ ID NO: 276) and 5′-TGTGGAATGTGTGCGAG-3′ (SEQ ID NO: 277), respectively, while the paired primer sequences for detecting the 3′ junction are F2 and R2: 5′-ATCATAATCAGCCATACCACA-3′ (SEQ ID NO: 278) and 5′-CATTTAGCAATCAACAGCAT-3′ (SEQ ID NO: 279), respectively. The U6 control group did not produce obvious and specific PCR product bands, whereas the experimental groups generated distinct PCR product bands of 577 bp and 723 bp (FIG. 6C). Gel extraction and recovery of the DNA bands corresponding to the 5′- and 3′-junctions amplified from the experimental group were performed. According to the instructions of Tsingke Biological's pClone007 Blunt Simple Vector Kit (#TSV-007BS), the PCR bands were cloned into the pUC19 vector, and clones were selected for Sanger sequencing. The results showed that none of the 5′ junctions in the selected 13 W/W, 17 W/C, 17 C/W, and 21 C/C clones achieved precise ligation. In contrast, 14 out of 15 W/W clones, 14 out of 16 W/C clones, 0 out of 17 C/W clones, and 16 out of 21 C/C clones had precisely ligated 3′ junctions. The precise ligation ratios for the W/C and C/C groups were 87.5% (14/16) and 76.2% (16/21), respectively (FIGS. 6C-6D). Since the donor with the W/C combination exhibited the highest gene knock-in efficiency (FIG. 6B), when both efficiency and precision are considered, the N-terminal gene knock-in efficiency and precision of the W/C donor combination are superior to those of other combinations. Specifically, the flanking regions of the donor DNA precursor should contain a Cpf1 target designed based on the target sequence of the gene of interest, with the PAM sequence of the upstream target located on Watson strand and that of the downstream target located on Crick strand.

### Example 3: C-Terminal Accurate Gene Knock-In Using Cpf1-Free Ends

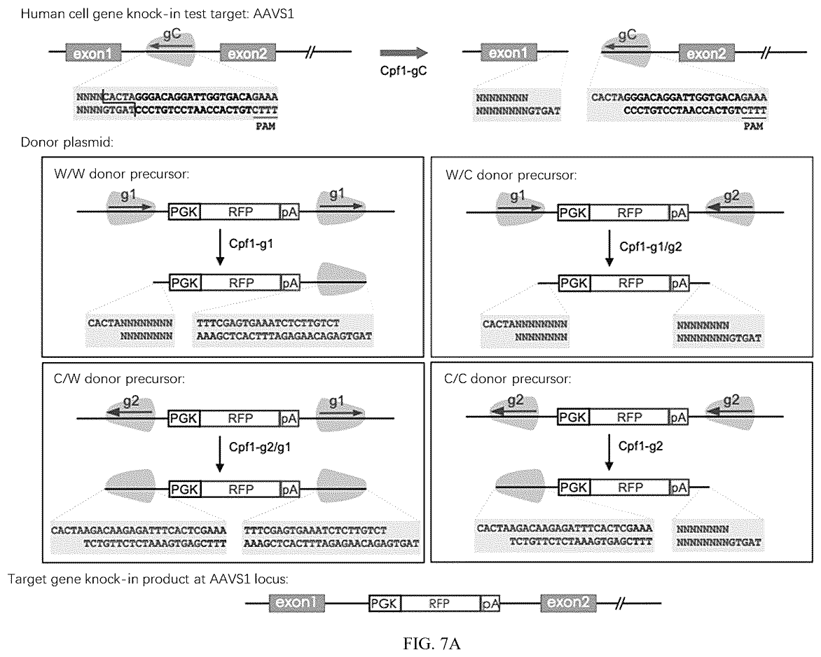

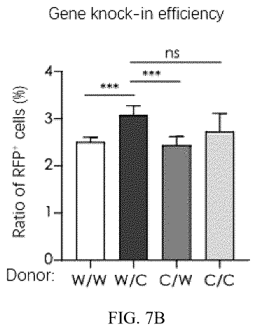

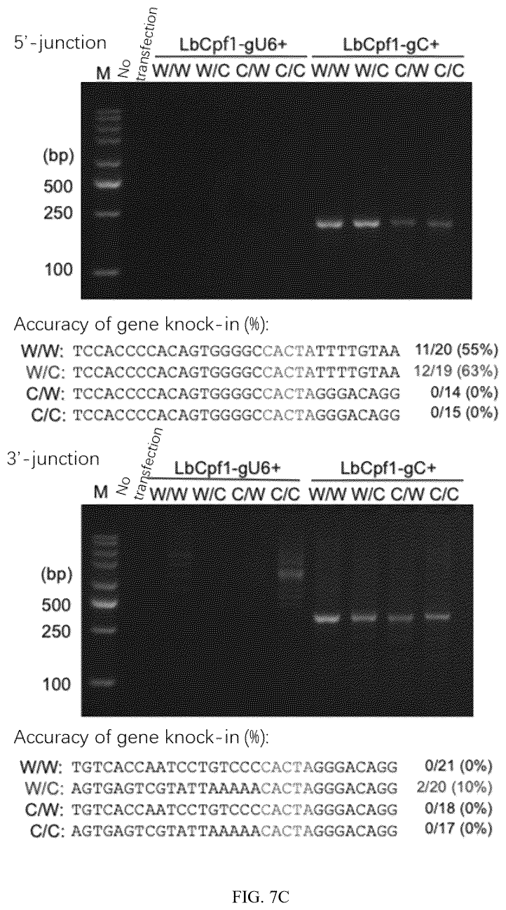

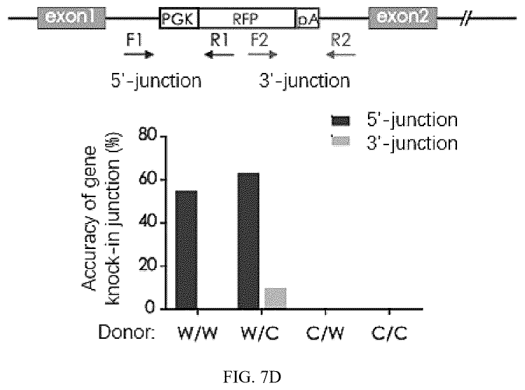

In this Example, the red fluorescent protein (RFP) gene was knocked into intron 1 of the AAVS1 locus (NCBI Gene ID: 17) in human HEK293T cells, ensuring precise connection between the C-terminus of the recipient gene insertion site and the RFP gene (FIG. 7A).

(1) Design and construct the gRNA (i.e., gC) for the Cpf1 target in intron 1 of the AAVS1 locus: To achieve precise connection between the C-terminus of the recipient gene insertion site and the RFP gene, design gC for the Cpf1 target in intron 1 of AAVS1. Its spacer sequence is 5′-TGTCACCAATCCTGTCCCTAGTG-3′ (SEQ ID NO: 280), and the PAM sequence (5′-TTTC-3′) is located on Crick strand. Cleavage at the Cpf1 target should generate an upstream end (Cpf1-free PAM-proximal end) with a 5′-overhang of 3′-GTGAT-5′ and a downstream end (Cpf1-retained PAM-distal end) with a 5′-overhang of 3′-ATCAC-5′ (FIG. 7A). Synthesize the aforementioned gC sgRNA sequence and ligate it into the pU6-LbCpf1-crRNA vector to form a plasmid expressing sgRNA.

(2) Design donor plasmids carrying cpf1 targets with four different pam direction combinations at both ends. W represents Watson strand; C represents Crick strand. The donor plasmid is composed of the AAVS1 upstream recognition sequence (PAM on the Watson or Crick strand)-PGK-RFP-pA expression cassette-AAVS1 downstream recognition sequence (PAM on the Watson or Crick strand). The PGK-RFP-pA expression cassette is identical to that in Example 2. The AAVS1 upstream and downstream recognition sequences can be either g1 in the W direction (5′-TGTCACCAATCCTGTCCCCACTA-3′ (SEQ ID NO: 281), PAM: 5′-TTTC-3′) or g2 in the C direction (5′-TGTCACCAATCCTGTCCCTAGTG-3′ (SEQ ID NO: 280), PAM: 5′-TTTC-3′). Thus, the combinations of the AAVS1 upstream and downstream recognition sequences include four types: W/W, W/C, C/W, and C/C. Cleavage of the donor precursor targets by Cpf1 generates a donor with two 5′-cohesive ends: the upstream end can be either the Cpf1-retained PAM-proximal end or the Cpf1-free PAM-distal end, with the 5′-overhang having a base composition of 3′-ATCAC-5′; the downstream end can be either the Cpf1-retained PAM-proximal end or the Cpf1-free PAM-distal end, with the 5′-overhang having a base composition of 3′-GTGAT-5′. Gene-synthesize the aforementioned AAVS1 upstream and downstream recognition sequences, and seamlessly ligate them with the 1567 bp red fluorescent protein (RFP) expression cassette (containing the PGK promoter and PolyA tail) obtained by PCR amplification using the Seamless Cloning Kit (Abclonal, Cat. No. RK21020) to obtain four combinations of donor plasmids (W/W, W/C, C/W, and C/C) (FIG. 7A). Specifically, the PGK-RFP-pA expression cassette is flanked by 27 bp Cpf1-gRNA target sequences containing PAM, forming the four PAM combinations (W/W, W/C, C/W, and C/C).

(3) Construct gRNA g1 and/or gRNA g2 for the Cpf1 targets at both ends of the donor plasmid. The spacer sequence of g1 is 5′-TGTCACCAATCCTGTCCCCACTA-33′ (SEQ ID NO: 281) with a PAM of 5′-TTTC-3′; the spacer sequence of g2 is 5′-TGTCACCAATCCTGTCCCTAGTG-3′ (SEQ ID NO: 280) with a PAM of 5′-TTTC-3′. Design and synthesize the aforementioned sgRNA sequences, then ligate them into the pU6-LbCpf1-crRNA vector to form plasmids expressing g1 and g2 (FIG. 7A).

(4) Detection of gene knock-in efficiency and accuracy. Using the lipofectamine 2000 liposome transfection method, transfect the four types of donor plasmids from step (2) into HEK293T cells, respectively, together with the gC expression plasmid targeting the genomic AAVS1 locus from step (1), the g1/g2 plasmids targeting the donor precursor loci from step (3), and the cpf1 expression plasmid. Transfection system (taking a 24-well plate as an example): each well contains 1.4×10⁵ cells, with a total transfected dna amount of 0.8 μg, including 0.4 μg of donor plasmid, 0.2 μg of Cpf1 expression plasmid, and 0.05 μg of each sgRNA (1 from step (1); 2 from step (3), which may be identical). A sgRNA blank control group (u6) was set up simultaneously, where all four sgRNAs were replaced with empty u6 plasmids without sgRNA. To eliminate systematic errors and improve the reliability and accuracy of the experiment, transfection efficiency correction is required. Therefore, the gene knock-in efficiency was corrected using the transfection efficiency of the green fluorescent protein (GFP) gene-specifically, the frequency of RFP cells measured by flow cytometry was multiplied by 100 and divided by the frequency of GFP cells. Results from three independent experiments showed that flow cytometry detection 23 days after transfection revealed almost no RFP cells in the u6 control group, while the frequencies of RFP cells generated by the four donors were 2.5% (W/W), 3.1% (W/C), 2.4% (C/W), and 2.7% (C/C) (FIG. 7B). Statistical analysis indicated that the donors with the W/C and C/C combinations exhibited relatively higher gene knock-in efficiency.

Collect cells from the U6 control group and the four experimental groups with different donors on day 3, then extract genomic DNA. PCR amplify the 5′ and 3′ junctions of the AAVS1 genomic target gene knock-in. The paired primer sequences for detecting the 5′ junction are F1 and R1, 5′-GTCACCTCTCACTCCTTTCA-3′ (SEQ ID NO: 282) and 5′-TGTGGAATGTGTGCGAG-3′ (SEQ ID NO: 277), respectively, while the paired primer sequences for detecting the 3′ junction are F2 and R2: 5′-CCACAACGAGGACTACACC-3′ (SEQ ID NO: 283) and 5′-CATCGTAAGCAAACCTTAGA-3′ (SEQ ID NO: 63), respectively.

The U6 control group did not produce obvious and specific PCR product bands, whereas the experimental groups generated distinct PCR product bands of 414 bp and 593 bp (FIG. 7C). Gel extraction and recovery of the DNA bands corresponding to the 5′- and 3′-junctions amplified from the experimental group were performed. According to the instructions of Tsingke Biological's pClone007 Blunt Simple Vector Kit (#TSV-007BS), the PCR bands were cloned into the pUC19 vector, and clones were selected for Sanger sequencing. The results showed that 11 out of 20 W/W clones, 12 out of 19 W/C clones, 0 out of 14 C/W clones, and 0 out of 15 C/C clones had precisely ligated 5′ junctions. In contrast, none of the 21 W/W clones, 18 out of 20 W/C clones, 18 C/W clones, and 17 C/C clones had precisely ligated 3′ junctions (FIG. 7C). In other words, the W/W and W/C groups exhibited high precise ligation ratios at the 5′ junction, reaching 55% (11/20) and 63.2% (12/19) respectively. At the 3′ junction, only the W/C group achieved precise knock-in, but the ratio was only 10% (2/20) (FIG. 7D). Therefore, only when the donor is in the W/C combination do both the efficiency and accuracy of C-terminal gene knock-in reach the highest, the flanking regions of the donor DNA precursor should contain a Cpf1 target designed based on the target sequence of the gene of interest, with the PAM sequence of the upstream target located on Watson strand and that of the downstream target located on Crick strand.

### Example 4: CANX-RFP Fusion Generated by Dual-End Accurate Gene Knock-In Using Cpf1-Free Ends

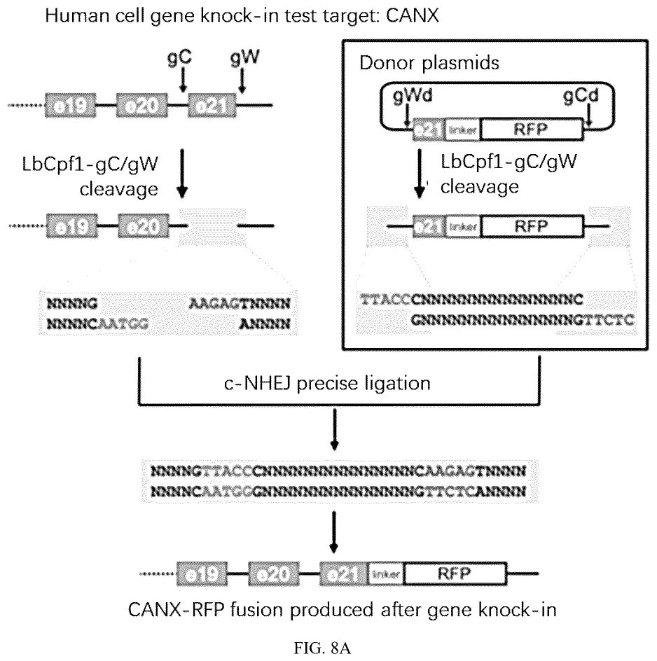

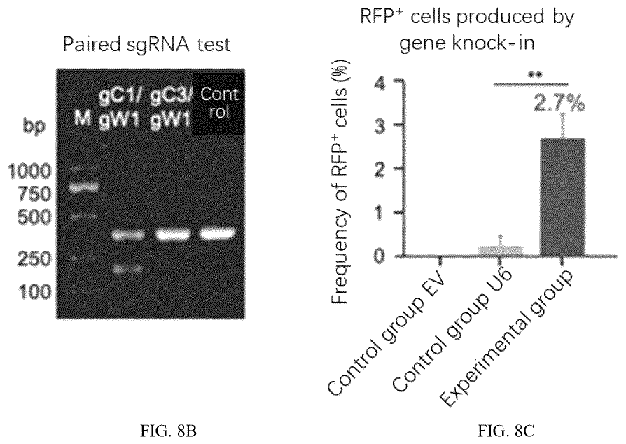

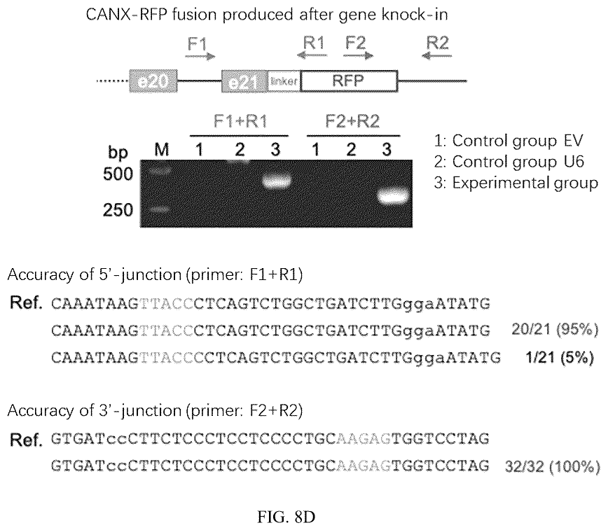

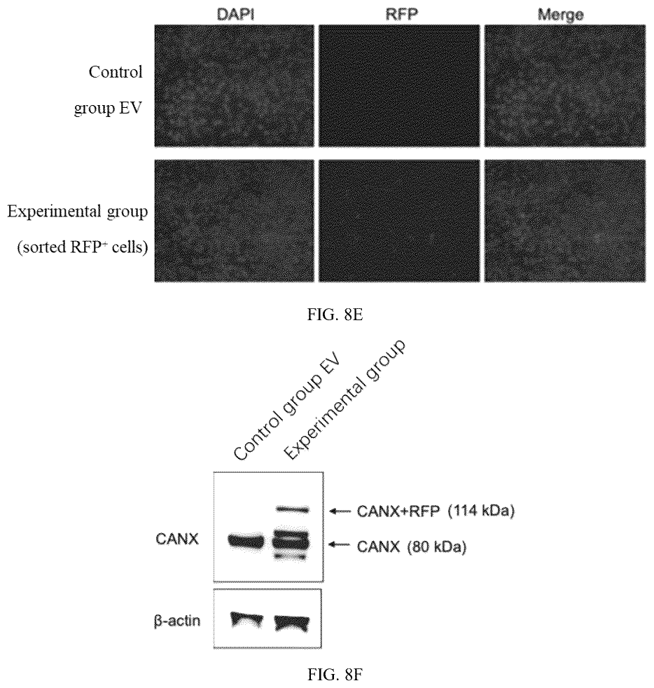

This Example uses Cpf1-free ends in human HEK293T cells, combined with the linker-RFP reporter gene, to achieve dual-end precise gene knock-in into the CANX gene (NCBI Gene ID: 821) for generating CANX-RFP fusion (FIG. 8A).

(1) Design and verification of the optimal paired Cpf1-sgRNA combination for CANX genomic targets: Three gRNAs (gC1, gC3, and gW1) were designed at adjacent positions upstream and downstream of the last exon (exon 21) of the CANX gene. The sequences of gC1 and gC3 are 5′-AAGATCAGCCAGACTGAGGGTAA-3′ (SEQ ID NO: 284) and 5′-ACTCTCTTCGTGGCTTTCTGTTT-3′ (SEQ ID NO: 285), respectively, with their PAM (5′-TTTC-3′) located Crick on strand. The sequence of is 5′-TTCTCCCTCCTCCCCTGCAAGAG-3′ (SEQ ID NO: 286), with its PAM (5′-TTTC-3′) located on Watson strand (FIG. 8A). The aforementioned sgRNA sequences were synthesized and ligated into the pU6-LbCpf1-crRNA vector to construct sgRNA expression plasmids. The paired sgRNA combinations are gC1 with gW1 and gC3 with gW1. These two combinations (gC1/gW1 or gC3/gW1) were transfected into HEK293T cells together with the Cpf1 expression plasmid. 72 hours after transfection, cellular genomic DNA was extracted, and target PCR amplification was performed using paired primers F1 and R1: 5′-CATAATTCCACCACCTCTG-3′ (SEQ ID NO: 287) and 5′-GTTACACAGACTAGTGTTCA-3′ (SEQ ID NO: 288). PCR products were analyzed by DNA gel electrophoresis. The results showed that the cleavage effect of the gC1/gW1 paired combination was the most prominent (FIG. 8B), so this pair of sgRNAs was selected for subsequent gene knock-in experiments. Moreover, simultaneous cleavage by the gC1/gW1 paired combination will delete the intervening sequence between the two targets, which is exon 21 of CANX.

(2) Design and construct the donor plasmid. The donor plasmid consists of the upstream recognition sequence of CANX exon 21 (PAM on Watson strand)-CANX exon 21-linker-RFP-downstream recognition sequence of exon 21 (PAM on Crick strand). The upstream and downstream recognition sequences of CANX exon 21 are gWd in the W direction (5′-AAGATCAGCCAGACTGAGTTACC-3′ (SEQ ID NO: 289), PAM: 5′-TTTC-3′) and gCd in the C direction (5′-TTCTCCCTCCTCCCCTGCCTCTT-3′ (SEQ ID NO: 290), PAM: 5′-TTTC-3′), respectively. Cleavage of the donor precursor targets by Cpf1 generates a donor with two 5′-cohesive ends: the 5′-overhang of the upstream end has a base composition of 3′-CCATT-5′, and the 5′-overhang of the downstream end has a base composition of 3′-TTCTC-5′. Both the upstream and downstream ends are Cpf1-free PAM-distal ends. The CANX exon 21 in this donor precursor is the sequence of CANX exon 21 that needs to be deleted in Step (1) and lacks the stop codon TGA. The flexible fusion protein linker is (GGGGS) 3. The coding sequence of the red fluorescent protein (RFP) gene is shown in SEQ ID NO: 2, derived from the pcDNA3-mRFP plasmid (purchased from Addgene, Cat. No. #13032). Gene-synthesize the aforementioned gWd and gCd recognition sequences, the CANX exon 21 without the stop codon TGA, and the flexible fusion protein linker. Seamlessly ligate them with the RFP (1567 bp) obtained by PCR amplification using the Seamless Cloning Kit (Abclonal, Cat. No. RK21020) to obtain the donor plasmid (SEQ ID NO: 3) (FIG. 8A).

(3) Construct gRNA gWd and gCd for the Cpf1 targets at both ends of the donor precursor. The spacer sequence of gWd is 5′-AAGATCAGCCAGACTGAGTTACC-3′ (SEQ ID NO: 289) with a PAM of 5′-TTTC-3′; the spacer sequence of gCd is 5′-TTCTCCCTCCTCCCCTGCCTCTT-3′ (SEQ ID NO: 290) with a PAM of 5′-TTTC-3′. Design and synthesize the aforementioned sgRNA sequences, then ligate them into the pU6-LbCpf1-crRNA vector to form plasmids expressing gWd and gCd (FIG. 8A).

(4) Detection of gene knock-in efficiency and accuracy. Using the Lipofectamine 2000 liposome transfection method, transfect one type of donor precursor plasmid from Step (2) into HEK293T cells, respectively, together with the paired gC/gW plasmids targeting the genomic locus of CANX exon 21 from Step (1), the gWd/gCd plasmids targeting the donor precursor loci from Step (3), and the Cpf1 expression plasmid. Transfection system (taking a 24-well plate as an example): each well contains 1.4×10⁵ cells, with a total transfected DNA amount of including 0.4 μg of donor plasmid, 0.2 μg of Cpf1 expression plasmid, and 0.05 μg of each sgRNA. A blank control group (EV) and a sgRNA blank control group (U6) were set up simultaneously. For the EV group, the donor plasmid, Cpf1 expression plasmid, and sgRNA expression plasmids were completely replaced with empty vectors. For the U6 group, all four sgRNAs were replaced with empty U6 plasmids without sgRNA. To eliminate systematic errors and improve the reliability and accuracy of the experiment, transfection efficiency correction is required. Therefore, the gene knock-in efficiency was corrected using the transfection efficiency of the green fluorescent protein (GFP) gene-specifically, the frequency of RFP cells measured by flow cytometry was multiplied by 100 and divided by the frequency of GFP cells. The results showed that flow cytometry detection 10 days after transfection revealed almost no RFP cells in the EV control group and U6 control group, while the frequency of RFP cells generated by the experimental group was 2.7% (FIG. 8C).

Collect cells from the EV control group, U6 control group, and experimental group (KI) on day 10, then extract genomic DNA. PCR amplify the 5′ and 3′ junctions of the gene knock-in at the CANX locus. The paired primer sequences for detecting the 5′ junction are F1 and R1: 5′-CATAATTCCACCACCTCTG-3′ (SEQ ID NO: 287) and 5′-CTCGGTCACCTTCAGCTTG-3′ (SEQ ID NO: 291), while the paired primer sequences for detecting the 3′ junction are F2 and R2 5′-CTGGACATCACCTCCCACA-3′ (SEQ ID NO: 292) and 5′-GTTACACAGACTAGTGTTCA-3′ (SEQ ID NO: 288). The results showed that only the experimental group could amplify the predicted 435 bp and 322 bp gene knock-in bands, whereas no gene knock-in bands were observed in the EV control group and U6 control group (FIG. 8D). Gel extraction and recovery of the 5′ and 3′ junction bands amplified from the experimental group were performed. According to the instructions of Tsingke Biological's pClone007 Blunt Simple Vector Kit (#TSV-007BS), the PCR bands were cloned into the pUC19 vector, and clones were selected for Sanger sequencing. The results showed that the precise knock-in ratio of the 5′ junction reached 95% (20/21), while the precise knock-in ratio of the 3′ junction reached 100% (32/32) (FIG. 8D).

Further sort the RFP cells on day 10, stain the cell nuclei with DAPI dye, and observe under an immunofluorescence microscope. RFP cells were detected in the experimental group, with the RFP protein localized in the cytoplasm, consistent with the cytoplasmic localization of CANX. In contrast, no RFP cells were observed in the EV control group (FIG. 8E). Meanwhile, Western blot analysis was performed on the sorted RFP cells from the experimental group. In addition to the wild type CANX protein (approximately 80 kDa), a CANX-RFP fusion protein with a molecular weight of approximately 114 kDa was detected in the experimental group. Only the wild type CANX protein was present in the EV control group (FIG. 8F). These results indicate that the use of Cpf1 free ends can improve dual-end precise gene knock-in to generate the CANX-RFP fusion protein. Specifically: the PAM of the upstream Cpf1 target of the target gene is located on C strand, and the PAM of the downstream Cpf1 target of the target gene is located on W strand; the PAM of the upstream Cpf1 target of the donor DNA precursor is located on W strand, and the PAM of the downstream Cpf1 target PAM of the donor DNA precursor is located on C strand.

### Example 5: Dual-End Accurate Gene Knock-In for PCNA-RFP Fusion Using Cpf1-Free Ends

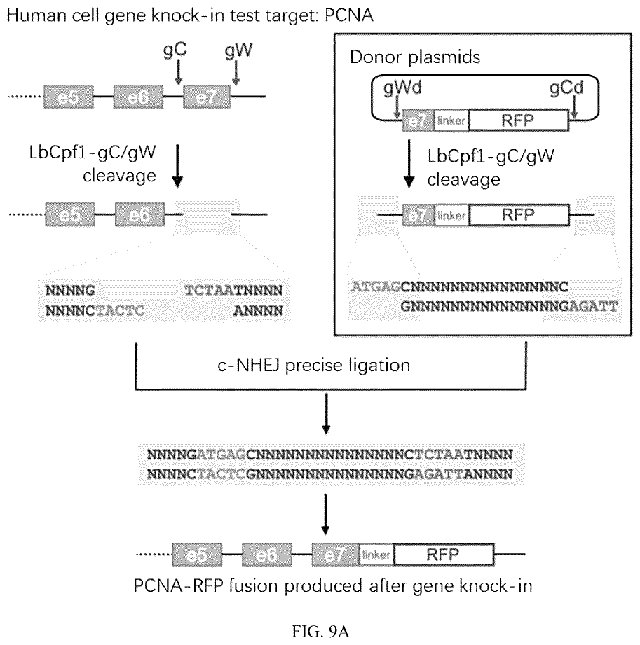

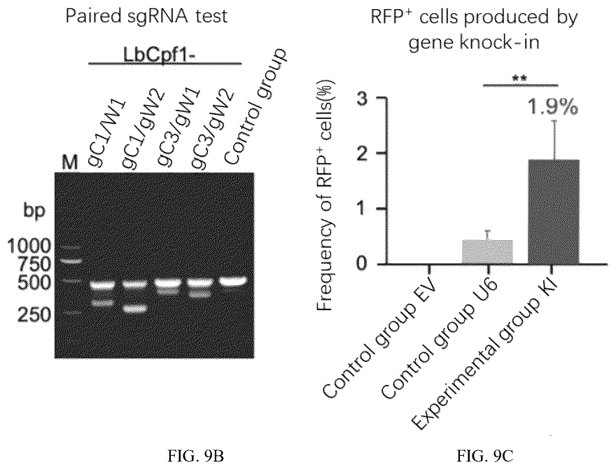

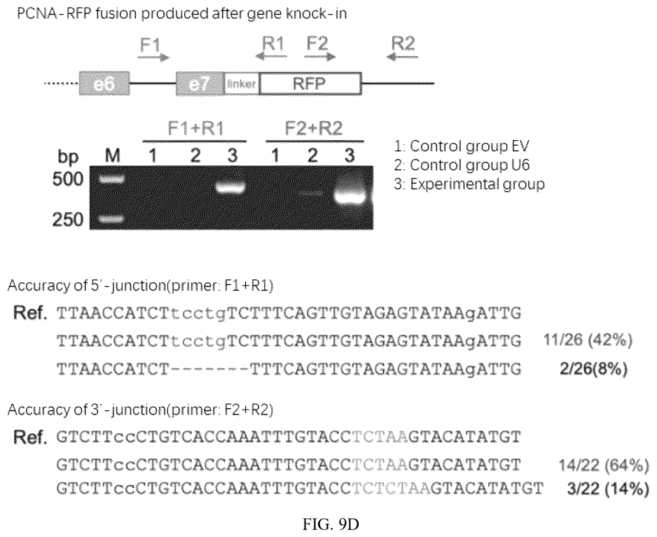

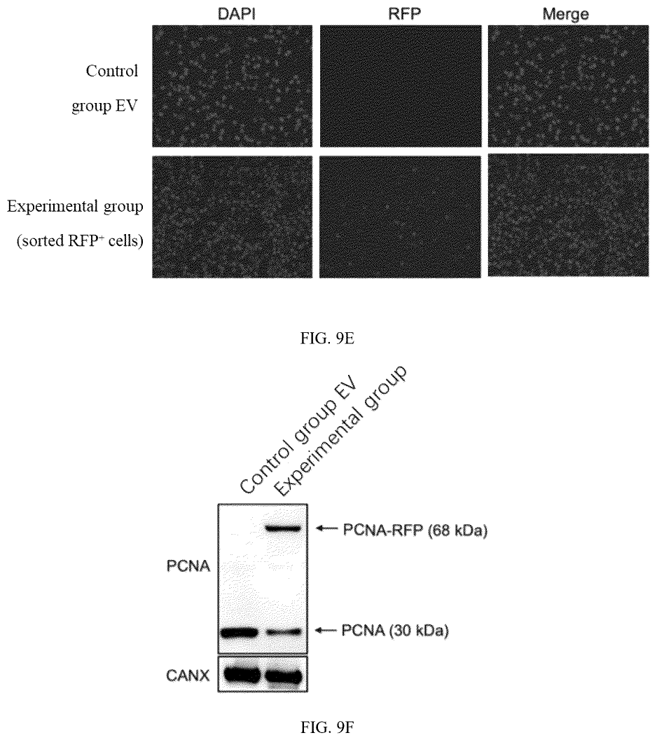

This Example uses Cpf1-free ends in human HEK293T cells, combined with the linker-RFP reporter gene, to achieve dual-end precise gene knock-in into the PCNA gene (NCBI Gene ID: 5111) for generating PCNA-RFP fusion (FIG. 9A).

(1) Design and verification of the optimal paired Cpf1-sgRNA combination for PCNA genomic targets: Four gRNAs (gC1, gC3, gW1, and gW2) were designed at adjacent positions upstream and downstream of the last exon (exon 7) of the PCNA gene. The sequences of gC1 and gC3 are 5′-TACTCTACAACTGAAAGACAGGA-3′ (SEQ ID NO: 293) and 5′-AGTGTCCCATATCCGCAATTTTA-3′ (SEQ ID NO: 294), respectively, with their PAM (5′-TTTA-3′) located on Crick strand. The sequences of gW1 and gW2 are 5′-AGAACTGCTTCTAAGATGCCAGC-3′ (SEQ ID NO: 295) (PAM: 5′-TTTG-3′) and 5′-TGTCACCAAATTTGTACCTCTAA-3′ (SEQ ID NO: 296) (PAM: 5′-TTTC-3′), respectively, with their PAM located on Watson strand (FIG. 9A). The aforementioned sgRNA sequences were synthesized and ligated into the pU6-LbCpf1-crRNA vector to construct sgRNA expression plasmids. The paired sgRNA combinations are gC1 with gW1, gC1 with gW2, gC3 with gW1, and gC3 with gW2. These four combinations (gC1/gW1, gC1/gW2, gC3/gW1, and gC3/gW2) were transfected into HEK293T cells together with the Cpf1 plasmid. 72 hours after transfection, cellular genomic DNA was extracted, and target PCR amplification was performed using paired primers F1 and R1 5′-CTCTCTTCAACGGTGACAC-3′ (SEQ ID NO: 297) and 5′-GATCTGACTTTGGACTTTATTC-3′ (SEQ ID NO: 298). PCR products were analyzed by DNA gel electrophoresis. The results showed that the cleavage effect of the gC1/gW2 paired combination was the most prominent (FIG. 9B), so this pair of sgRNAs was selected for subsequent gene knock-in experiments.

(2) Design and construct the donor plasmid. The donor plasmid consists of the upstream recognition sequence of PCNA exon 7 (PAM on Watson strand)-PCNA exon 7-linker-RFP-downstream recognition sequence of PCNA exon 7 (PAM on Crick strand). The upstream and downstream recognition sequences of PCNA exon 7 are gWd in the W direction (spacer sequence: 5′-ACTCTACAACTGAAAGATCCTG-3′ (SEQ ID NO: 299), PAM: 5′-TTTA-3′) and gCd in the C direction (spacer sequence: 5′-TGTCACCAAATTTGTACCTTAGA-3′ (SEQ ID NO: 300), PAM: 5′-TTTC-3′), respectively. Cleavage of the donor precursor targets by Cpf1 generates a donor with two 5′-cohesive ends: the 5′-overhang of the upstream end has a base composition of 3′-GTCCT-5′, and the 5′-overhang of the downstream end has a base composition of 3′-AGATT-5′. Both the upstream and downstream ends are Cpf1-free PAM-distal ends. The PCNA exon 7 in this donor plasmid is the sequence of PCNA exon 7 that needs to be deleted in Step (1) and lacks the stop codon TAG. The flexible fusion protein linker is (GGGGS) 3. The sequence of the red fluorescent protein (RFP) gene is derived from the pcDNA3-mRFP plasmid (purchased from Addgene, Cat. No. #13032). Gene-synthesize the aforementioned gWd and gCd recognition sequences, the PCNA exon 7 without the stop codon TAG, and the flexible fusion protein linker. Seamlessly ligate them with the 1567 bp RFP obtained by PCR amplification using the Seamless Cloning Kit (Abclonal, Cat. No. RK21020) to obtain the donor plasmid (SEQ ID NO: 4) (FIG. 9A).

(3) Construct gRNAs gWd and gCd for the Cpf1 targets at both ends of the donor precursor. The spacer sequence of gWd is 5′-ACTCTACAACTGAAAGATCCTG-3′ (SEQ ID NO: 299) with a PAM of 5′-TTTA-3′; the spacer sequence of gCd is 5′-TGTCACCAAATTTGTACCTTAGA-3′ (SEQ ID NO: 300) with a PAM of 5′-TTTC-3′. Design and synthesize the aforementioned sgRNA sequences, then ligate them into the pU6-LbCpf1-crRNA vector to form plasmids expressing gWd and gCd (FIG. 9A).

(4) Detection of gene knock-in efficiency and accuracy. Using the Lipofectamine 2000 liposome transfection method, transfect one type of donor precursor plasmid from Step (2) into HEK293T cells, together with the paired gC1/gW2 plasmids targeting the genomic locus of PCNA exon 7 from Step (1), the gWd/gCd plasmids targeting the donor precursor loci from Step (3), and the Cpf1 expression plasmid. Transfection system (taking a 24-well plate as an example): each well contains 1.4×10⁵ cells, with a total transfected DNA amount of 0.8 μg. This includes 0.4 μg of donor plasmid, 0.2 μg of Cpf1 expression plasmid, and 0.05 μg of each sgRNA. A blank control group (EV) and a sgRNA blank control group (U6) were set up simultaneously. For the EV group, the donor plasmid, Cpf1 expression plasmid, and sgRNA expression plasmids were completely replaced with empty vectors. For the U6 group, all four sgRNAs were replaced with empty U6 plasmids without sgRNA. To eliminate systematic errors and improve the reliability and accuracy of the experiment, transfection efficiency correction is required. Therefore, the gene knock-in efficiency was corrected using the transfection efficiency of the green fluorescent protein (GFP) gene-specifically, the frequency of RFP cells measured by flow cytometry was multiplied by 100 and divided by the frequency of GFP cells. The results showed that flow cytometry detection 10 days after transfection revealed almost no RFP cells in the EV control group and U6 control group, while the frequency of RFP cells generated by the experimental group was 1.9% (FIG. 9C).

Collect cells from the EV control group, U6 control group, and experimental group (KI) on day 10, then extract genomic DNA. PCR amplify the 5′ and 3′ junctions of the gene knock-in at the PCNA locus. The paired primer sequences for detecting the 5′ junction are F1 (5′-TACTCTACAACTGAAAGATCCTG-3′ (SEQ ID NO: 299)) and R1 (5′-CTCGGTCACCTTCAGCTTG-3′ (SEQ ID NO: 291)), while the paired primer sequences for detecting the 3′ junction are F2 (5′-CTGGACATCACCTCCCACA-3′ (SEQ ID NO: 292)) and R2 (5′-TGTCACCAAATTTGTACCTTAGA-3′ (SEQ ID NO: 300)). The results showed that only the experimental group could amplify the predicted 4190 bp and 364 bp gene knock-in bands, whereas no gene knock-in bands were observed in the EV control group and U6 control group (FIG. 9D). Gel extraction and recovery of the 5′ and 3′ junction bands amplified from the experimental group were performed. According to the instructions of Tsingke Biological's pClone007 Blunt Simple Vector Kit (#TSV-007BS), the PCR bands were cloned into the pUC19 vector, and clones were selected for Sanger sequencing. The results showed that the precise knock-in ratio of the 5′ junction reached 42% (11/26), while the precise knock-in ratio of the 3′ junction reached 64% (14/22) (FIG. 9D).

Further sort the RFP⁺ cells on day 10, stain the cell nuclei with DAPI dye, and observe under an immunofluorescence microscope. RFP⁺ cells were detected in the experimental group, with the RFP protein localized in the cell nucleus, consistent with the nuclear localization of PCNA. In contrast, no RFP⁺ cells were observed in the EV control group (FIG. 9E). Meanwhile, Western blot analysis was performed on the sorted RFP⁺ cells from the experimental group. In addition to the wild type PCNA protein (approximately 30 kDa), a PCNA-RFP fusion protein with a molecular weight of approximately 68 kDa was detected in the experimental group. Only the wild type PCNA protein was present in the EV control group (FIG. 9F). These results indicate that the use of Cpf1-free ends can improve dual-end precise gene knock-in to generate the PCNA-RFP fusion protein.

Sequence information

| SEQ ID | |
|---|---|
| NO: | Specific sequence |
| 1 | ATGGTGAGCAAGGGCGAGGAGCTGTTCACCGGGGTGGTGCCCATCCTGG |
| | TCGAGCTGGACGGCGACGTAAACGGCCACAAGTTCAGCGTGTCCGGCGA |
| | GGGCGAGGGCGATGCCACCTACGGCAAGCTGACCCTGAAGTTCATCTGC |
| | ACCACCGGCAAGCTGCCCGTGCCCTGGCCCACCCTCGTGACCACCCTGA |
| | CCTACGGCGTGCAGTGCTTCAGCCGCTACCCCGACCACATGAAGCAGCA |
| | CGACTTCTTCAAGTCCGCCATGCCCGAAGGCTACGTCCAGGAGCGCACC |
| | ATCTTCTTCAAGGACGACGGCAACTACAAGACCCGCGCCGAGGTGAAGT |
| | TCGAGGGCGACACCCTGGTGAACCGCATCGAGCTGAAGGGCATCGTAGG |
| | GATAACAGGGTAATCAAGGAGGACGGCAACATCCTGGGGCACAAGCTG |
| | GAGTACAACTACAACAGCCACAACGTCTATATCATGGCCGACAAGCAGA |
| | AGAACGGCATCAAGGTGAACTTCAAGATCCGCCACAACATCGAGGACG |
| | GCAGCGTGCAGCTCGCCGACCACTACCAGCAGAACACCCCCATCGGCGA |
| | CGGCCCCGTGCTGCTGCCCGACAACCACTACCTGAGCACCCAGTCCGCC |
| | CTGAGCAAAGACCCCAACGAGAAGCGCGATCACATGGTCCTGCTGGAGT |
| | TCGTGACCGCCGCCGGGATCACTCTCGGCATGGACGAGCTGTACAAGTA |
| | A |
| 2 | GGGGGTAGGGGAGGCGCTTTTCCAAGGCAGTCTGAGCATGCGCTTAGCA |
| | GCCCCGCTGGCACTTGGCGCTACACAAGTGGCCTYTGGCCTCGCACACA |
| | TTCCACATCCACCGGTAGGCGCCAACCGGCTCCGTTCTTTGGTGGCCCCT |
| | TCGCGCCACCTTCTWCTCCTCCCCTAGTCAGGAAGTTCCCCCCCGCCCCG |
| | CAGCTCGCGTCGTSAGGACGTGACAAATGGAAGTAGCACGTCTCACTAG |
| | TCTCGTCAGATGGACAGCACCGCTGAGCAATGGAAGCGGGTAGGCCTTT |
| | GGGGCAGCGGCCAATAGCAGCTTTGCTCCTTCGCTTTCTGGGCTCAGAG |
| | GCTGGGAAGGGGTGGGTCCGGGGGCGGGCTCAGGGGGGGGCTCAGGGG |
| | CGGGGCGGGCGCCCGAAGGTCCTCCGGAGGCCCGGCATTCTGCACGCTT |
| | CAAAAGCGCACGTCTGCCGCGCTGTTCTCCTCTTCCTCATCTCCGGGCCT |
| | TTCGACCTGCAGGTCCTCGCCATGGATCCTGCTAGCATGGTGAGCAAGG |
| | GCGAGGAGAATAACATGGCCGTCATCAAGGAGTTCATGCGCTTCAAGGT |
| | GCGCATGGAGGGCTCCGTGAACGGCCACGAGTTCGAGATCGAGGGCGA |
| | GGGCGAGGGCCGCCCCTACGAGGGCACCCAGACCGCCAAGCTGAAGGT |
| | GACCGAGGGTGGCCCCCTGCCCTTCGCCTGGGACATCCTGTCCCCTCAGT |
| | TCATGTACGGCTCCAAGGCCTACGTGAAGCACCCCGCCGACATCCCCGA |
| | CTACTTGAAGCTGTCCTTCCCCGAGGGCTTCAAGTGGGAGCGCGTGATG |
| | AACTTCGAGGACGGCGGCGTGGTGACCGTGACCCAGGACTCCTCCCTGC |
| | AGGACGGCGAGTTCATCTACAAGGTGAAGCTGCGCGGCACCAACTTCCC |
| | CTCCGACGGCCCCGTAATGCAGAAGAAGACCATGGGCTGGGAGGCCTCC |
| | TCCGAGCGGATGTACCCCGAGGACGGCGCCCTGAAGGGCGAGATCAAG |
| | ATGAGGCTGAAGCTGAAGGACGGTGGCCACTACGACGCCGAGGTCAAG |
| | ACCACCTACATGGCCAAGAAGCCCGTGCAGCTGCCCGGCGCCTACAAGA |
| | CCGACATCAAGCTGGACATCACCTCCCACAACGAGGACTACACCATCGT |
| | GGAACAGTACGAGCGCGCCGAGGGCCGCCACTCCACCGGCGGCATGGA |
| | CGAGCTGTACAAGAGATCTCGAGCTCAAGCTTCGAATTCTGCAGTCGAC |
| | GGTACCGCGGGCCCGGGATCCACCGGATCTAGATAACTGATCATAATCA |
| | GCCATACCACATTTGTAGAGGTTTTACTTGCTTTAAAAAACCTCCCACAC |
| | CTCCCCCTGAACCTGAAACATAAAATGAATGCAATTGTTGTTGTTAACTT |
| | GTTTATTGCAGCTTATAATGGTTACAAATAAAGCAATAGCATCACAAATT |
| | TCACAAATAAAGCATTTTTTTCACTGCATTCTAGTTGTGGTTTGTCCAAA |
| | CTCATCAATGTATCTTACGCGCCCCTATAGTGAGTCGTATTAAAAA |
| 3 | TTTCAAGATCAGCCAGACTGAGTTACCCTCAGTCTGGCTGATCTTGGGAA |
| | TATGGTGAACTTTTATTATTTTTTAAATGTGCTAATTATAATGAATTTCTT |
| | TTCAATCAATAGGAGGATGAAATTTTGAACAGATCACCAAGAAACAGAA |
| | AGCCACGAAGAGAGGGCGGTGGCGGTAGTGGTGGCGGCGGCAGCGGTG |
| | GCGGTGGTAGCATGGTGAGCAAGGGCGAGGAGAATAACATGGCCGTCA |
| | TCAAGGAGTTCATGCGCTTCAAGGTGCGCATGGAGGGCTCCGTGAACGG |
| | CCACGAGTTCGAGATCGAGGGCGAGGGCGAGGGCCGCCCCTACGAGGG |
| | CACCCAGACCGCCAAGCTGAAGGTGACCGAGGGTGGCCCCCTGCCCTTC |
| | GCCTGGGACATCCTGTCCCCTCAGTTCATGTACGGCTCCAAGGCCTACGT |
| | GAAGCACCCCGCCGACATCCCCGACTACTTGAAGCTGTCCTTCCCCGAG |
| | GGCTTCAAGTGGGAGCGCGTGATGAACTTCGAGGACGGCGGCGTGGTGA |
| | CCGTGACCCAGGACTCCTCCCTGCAGGACGGCGAGTTCATCTACAAGGT |
| | GAAGCTGCGCGGCACCAACTTCCCCTCCGACGGCCCCGTAATGCAGAAG |
| | AAGACCATGGGCTGGGAGGCCTCCTCCGAGCGGATGTACCCCGAGGACG |
| | GCGCCCTGAAGGGCGAGATCAAGATGAGGCTGAAGCTGAAGGACGGTG |
| | GCCACTACGACGCCGAGGTCAAGACCACCTACATGGCCAAGAAGCCCGT |
| | GCAGCTGCCCGGCGCCTACAAGACCGACATCAAGCTGGACATCACCTCC |
| | CACAACGAGGACTACACCATCGTGGAACAGTACGAGCGCGCCGAGGGC |
| | CGCCACTCCACCGGCGGCATGGACGAGCTGTACAAGAGATCTCGAGCTC |
| | AAGCTTCGAATTCTGCAGTCGACGGTACCGCGGGCCCGGGATCCACCGG |
| | ATCTAGATAAAACAATCTTAAGAGCTTGATCTGTGATACCTTCTCCCTCC |
| | TCCCCTGCAAGAGGCAGGGGAGGAGGGAGAAGAAAGATATCGAATTCT |
| | ACCCTTCTGAGGCGGAAAGAACCAGCTGGGGCTCGAGGGGGGGCCCGG |
| | TACCCAGCTTTTGTTCCCTTTAGTGAGGGTTAATTGCGCGCTTGGCGTAA |
| | TCATGGTCATAGCTGTTTCCTGTGTGAAATTGTTATCCGCTCACAATTCC |
| | ACACAACATACGAGCCGGAAGCATAAAGTGTAAAGCCTGGGGTGCCTA |
| | ATGAGTGAGCTAACTCACATTAATTGCGTTGCGCTCACTGCCCGCTTTCC |
| | AGTCGGGAAACCTGTCGTGCCAGCTGCATTAATGAATCGGCCAACGCGC |
| | GGGGAGAGGCGGTTTGCGTATTGGGCGCTCTTCCGCTTCCTCGCTCACTG |
| | ACTCGCTGCGCTCGGTCGTTCGGCTGCGGCGAGCGGTATCAGCTCACTCA |
| | AAGGCGGTAATACGGTTATCCACAGAATCAGGGGATAACGCAGGAAAG |
| | AACATGTGAGCAAAAGGCCAGCAAAAGGCCAGGAACCGTAAAAAGGCC |
| | GCGTTGCTGGCGTTTTTCCATAGGCTCCGCCCCCCTGACGAGCATCACAA |
| | AAATCGACGCTCAAGTCAGAGGTGGCGAAACCCGACAGGACTATAAAG |
| | ATACCAGGCGTTTCCCCCTGGAAGCTCCCTCGTGCGCTCTCCTGTTCCGA |
| | CCCTGCCGCTTACCGGATACCTGTCCGCCTTTCTCCCTTCGGGAAGCGTG |
| | GCGCTTTCTCATAGCTCACGCTGTAGGTATCTCAGTTCGGTGTAGGTCGT |
| | TCGCTCCAAGCTGGGCTGTGTGCACGAACCCCCCGTTCAGCCCGACCGCT |
| | GCGCCTTATCCGGTAACTATCGTCTTGAGTCCAACCCGGTAAGACACGA |
| | CTTATCGCCACTGGCAGCAGCCACTGGTAACAGGATTAGCAGAGCGAGG |
| | TATGTAGGCGGTGCTACAGAGTTCTTGAAGTGGTGGCCTAACTACGGCT |
| | ACACTAGAAGGACAGTATTTGGTATCTGCGCTCTGCTGAAGCCAGTTAC |
| | CTTCGGAAAAAGAGTTGGTAGCTCTTGATCCGGCAAACAAACCACCGCT |
| | GGTAGCGGTGGTTTTTTTGTTTGCAAGCAGCAGATTACGCGCAGAAAAA |
| | AAGGATCTCAAGAAGATCCTTTGATCTTTTCTACGGGGTCTGACGCTCAG |
| | TGGAACGAAAACTCACGTTAAGGGATTTTGGTCATGAGATTATCAAAAA |
| | GGATCTTCACCTAGATCCTTTTAAATTAAAAATGAAGTTTTAAATCAATC |
| | TAAAGTATATATGAGTAAACTTGGTCTGACAGTTACCAATGCTTAATCAG |
| | TGAGGCACCTATCTCAGCGATCTGTCTATTTCGTTCATCCATAGTTGCCT |
| | GACTCCCCGTCGTGTAGATAACTACGATACGGGAGGGCTTACCATCTGG |
| | CCCCAGTGCTGCAATGATACCGCGAGACCCACGCTCACCGGCTCCAGAT |
| | TTATCAGCAATAAACCAGCCAGCCGGAAGGGCCGAGCGCAGAAGTGGT |
| | CCTGCAACTTTATCCGCCTCCATCCAGTCTATTAATTGTTGCCGGGAAGC |
| | TAGAGTAAGTAGTTCGCCAGTTAATAGTTTGCGCAACGTTGTTGCCATTG |
| | CTACAGGCATCGTGGTGTCACGCTCGTCGTTTGGTATGGCTTCATTCAGC |
| | TCCGGTTCCCAACGATCAAGGCGAGTTACATGATCCCCCATGTTGTGCAA |
| | AAAAGCGGTTAGCTCCTTCGGTCCTCCGATCGTTGTCAGAAGTAAGTTGG |
| | CCGCAGTGTTATCACTCATGGTTATGGCAGCACTGCATAATTCTCTTACT |
| | GTCATGCCATCCGTAAGATGCTTTTCTGTGACTGGTGAGTACTCAACCAA |
| | GTCATTCTGAGAATAGTGTATGCGGCGACCGAGTTGCTCTTGCCCGGCGT |
| | CAATACGGGATAATACCGCGCCACATAGCAGAACTTTAAAAGTGCTCAT |
| | CATTGGAAAACGTTCTTCGGGGCGAAAACTCTCAAGGATCTTACCGCTG |
| | TTGAGATCCAGTTCGATGTAACCCACTCGTGCACCCAACTGATCTTCAGC |
| | ATCTTTTACTTTCACCAGCGTTTCTGGGTGAGCAAAAACAGGAAGGCAA |
| | AATGCCGCAAAAAAGGGAATAAGGGCGACACGGAAATGTTGAATACTC |
| | ATACTCTTCCTTTTTCAATATTATTGAAGCATTTATCAGGGTTATTGTCTC |
| | ATGAGCGGATACATATTTGAATGTATTTAGAAAAATAAACAAATAGGGG |
| | TTCCGCGCACATTTCCCCGAAAAGTGCCACCTGACGCGCCCTGTAGCGG |
| | CGCATTAAGCGCGGCGGGTGTGGTGGTTACGCGCAGCGTGACCGCTACA |
| | CTTGCCAGCGCCCTAGCGCCCGCTCCTTTCGCTTTCTTCCCTTCCTTTCTC |
| | GCCACGTTCGCCGGCTTTCCCCGTCAAGCTCTAAATCGGGGGCTCCCTTT |
| | AGGGTTCCGATTTAGTGCTTTACGGCACCTCGACCCCAAAAAACTTGATT |
| | AGGGTGATGGTTCACGTAGTGGGCCATCGCCCTGATAGACGGTTTTTCGC |
| | CCTTTGACGTTGGAGTCCACGTTCTTTAATAGTGGACTCTTGTTCCAAAC |
| | TGGAACAACACTCAACCCTATCTCGGTCTATTCTTTTGATTTATAAGGGA |
| | TTTTGCCGATTTCGGCCTATTGGTTAAAAAATGAGCTGATTTAACAAAAA |
| | TTTAACGCGAATTTTAACAAAATATTAACGCTTACAATTTCCATTCGCCA |
| | TTCAGGCTGCGCAACTGTTGGGAAGGGCGATCGGTGCGGGCCTCTTCGC |
| | TATTACGCCAGCTGCGGCCGC |
| 4 | TTTATACTCTACAACTGAAAGATCCTGTCTTTCAGTTGTAGAGTATAAGA |
| | TTGCGGATATGGGACACTTAAAATACTACTTGGCTCCCAAGATCGAGGA |
| | TGAAGAAGGATCTGGCGGTGGCGGTAGTGGTGGCGGCGGCAGCGGTGG |
| | CGGTGGTAGCATGGTGAGCAAGGGCGAGGAGAATAACATGGCCGTCAT |
| | CAAGGAGTTCATGCGCTTCAAGGTGCGCATGGAGGGCTCCGTGAACGGC |
| | CACGAGTTCGAGATCGAGGGCGAGGGCGAGGGCCGCCCCTACGAGGGC |
| | ACCCAGACCGCCAAGCTGAAGGTGACCGAGGGTGGCCCCCTGCCCTTCG |
| | CCTGGGACATCCTGTCCCCTCAGTTCATGTACGGCTCCAAGGCCTACGTG |
| | AAGCACCCCGCCGACATCCCCGACTACTTGAAGCTGTCCTTCCCCGAGG |
| | GCTTCAAGTGGGAGCGCGTGATGAACTTCGAGGACGGCGGCGTGGTGAC |
| | CGTGACCCAGGACTCCTCCCTGCAGGACGGCGAGTTCATCTACAAGGTG |
| | AAGCTGCGCGGCACCAACTTCCCCTCCGACGGCCCCGTAATGCAGAAGA |
| | AGACCATGGGCTGGGAGGCCTCCTCCGAGCGGATGTACCCCGAGGACGG |
| | CGCCCTGAAGGGCGAGATCAAGATGAGGCTGAAGCTGAAGGACGGTGG |
| | CCACTACGACGCCGAGGTCAAGACCACCTACATGGCCAAGAAGCCCGTG |
| | CAGCTGCCCGGCGCCTACAAGACCGACATCAAGCTGGACATCACCTCCC |
| | ACAACGAGGACTACACCATCGTGGAACAGTACGAGCGCGCCGAGGGCC |
| | GCCACTCCACCGGCGGCATGGACGAGCTGTACAAGAGATCTCGAGCTCA |
| | AGCTTCGAATTCTGCAGTCGACGGTACCGCGGGCCCGGGATCCACCGGA |
| | TCTAGATAAGCATTCTTAAAATTCAAGAAAATAAAACTAAGCTCTTTGA |
| | GAACTGCTTCTAAGATGCCAGCATATACTGAAGTCTTCCCTGTCACCAAA |
| | TTTGTACCTCTAAGGTACAAATTTGGTGACAGAAAGATATCGAATTCTAC |
| | CCTTCTGAGGCGGAAAGAACCAGCTGGGGCTCGAGGGGGGGCCCGGTA |
| | CCCAGCTTTTGTTCCCTTTAGTGAGGGTTAATTGCGCGCTTGGCGTAATC |
| | ATGGTCATAGCTGTTTCCTGTGTGAAATTGTTATCCGCTCACAATTCCAC |
| | ACAACATACGAGCCGGAAGCATAAAGTGTAAAGCCTGGGGTGCCTAATG |
| | AGTGAGCTAACTCACATTAATTGCGTTGCGCTCACTGCCCGCTTTCCAGT |
| | CGGGAAACCTGTCGTGCCAGCTGCATTAATGAATCGGCCAACGCGCGGG |
| | GAGAGGCGGTTTGCGTATTGGGCGCTCTTCCGCTTCCTCGCTCACTGACT |
| | CGCTGCGCTCGGTCGTTCGGCTGCGGCGAGCGGTATCAGCTCACTCAAA |
| | GGCGGTAATACGGTTATCCACAGAATCAGGGGATAACGCAGGAAAGAA |
| | CATGTGAGCAAAAGGCCAGCAAAAGGCCAGGAACCGTAAAAAGGCCGC |
| | GTTGCTGGCGTTTTTCCATAGGCTCCGCCCCCCTGACGAGCATCACAAAA |
| | ATCGACGCTCAAGTCAGAGGTGGCGAAACCCGACAGGACTATAAAGAT |
| | ACCAGGCGTTTCCCCCTGGAAGCTCCCTCGTGCGCTCTCCTGTTCCGACC |
| | CTGCCGCTTACCGGATACCTGTCCGCCTTTCTCCCTTCGGGAAGCGTGGC |
| | GCTTTCTCATAGCTCACGCTGTAGGTATCTCAGTTCGGTGTAGGTCGTTC |
| | GCTCCAAGCTGGGCTGTGTGCACGAACCCCCCGTTCAGCCCGACCGCTG |
| | CGCCTTATCCGGTAACTATCGTCTTGAGTCCAACCCGGTAAGACACGACT |
| | TATCGCCACTGGCAGCAGCCACTGGTAACAGGATTAGCAGAGCGAGGTA |
| | TGTAGGCGGTGCTACAGAGTTCTTGAAGTGGTGGCCTAACTACGGCTAC |
| | ACTAGAAGGACAGTATTTGGTATCTGCGCTCTGCTGAAGCCAGTTACCTT |
| | CGGAAAAAGAGTTGGTAGCTCTTGATCCGGCAAACAAACCACCGCTGGT |
| | AGCGGTGGTTTTTTTGTTTGCAAGCAGCAGATTACGCGCAGAAAAAAAG |
| | GATCTCAAGAAGATCCTTTGATCTTTTCTACGGGGTCTGACGCTCAGTGG |
| | AACGAAAACTCACGTTAAGGGATTTTGGTCATGAGATTATCAAAAAGGA |
| | TCTTCACCTAGATCCTTTTAAATTAAAAATGAAGTTTTAAATCAATCTAA |
| | AGTATATATGAGTAAACTTGGTCTGACAGTTACCAATGCTTAATCAGTGA |
| | GGCACCTATCTCAGCGATCTGTCTATTTCGTTCATCCATAGTTGCCTGAC |
| | TCCCCGTCGTGTAGATAACTACGATACGGGAGGGCTTACCATCTGGCCC |
| | CAGTGCTGCAATGATACCGCGAGACCCACGCTCACCGGCTCCAGATTTA |
| | TCAGCAATAAACCAGCCAGCCGGAAGGGCCGAGCGCAGAAGTGGTCCT |
| | GCAACTTTATCCGCCTCCATCCAGTCTATTAATTGTTGCCGGGAAGCTAG |
| | AGTAAGTAGTTCGCCAGTTAATAGTTTGCGCAACGTTGTTGCCATTGCTA |
| | CAGGCATCGTGGTGTCACGCTCGTCGTTTGGTATGGCTTCATTCAGCTCC |
| | GGTTCCCAACGATCAAGGCGAGTTACATGATCCCCCATGTTGTGCAAAA |
| | AAGCGGTTAGCTCCTTCGGTCCTCCGATCGTTGTCAGAAGTAAGTTGGCC |
| | GCAGTGTTATCACTCATGGTTATGGCAGCACTGCATAATTCTCTTACTGT |
| | CATGCCATCCGTAAGATGCTTTTCTGTGACTGGTGAGTACTCAACCAAGT |
| | CATTCTGAGAATAGTGTATGCGGCGACCGAGTTGCTCTTGCCCGGCGTCA |
| | ATACGGGATAATACCGCGCCACATAGCAGAACTTTAAAAGTGCTCATCA |
| | TTGGAAAACGTTCTTCGGGGCGAAAACTCTCAAGGATCTTACCGCTGTTG |
| | AGATCCAGTTCGATGTAACCCACTCGTGCACCCAACTGATCTTCAGCATC |
| | TTTTACTTTCACCAGCGTTTCTGGGTGAGCAAAAACAGGAAGGCAAAAT |
| | GCCGCAAAAAAGGGAATAAGGGCGACACGGAAATGTTGAATACTCATA |
| | CTCTTCCTTTTTCAATATTATTGAAGCATTTATCAGGGTTATTGTCTCATG |
| | AGCGGATACATATTTGAATGTATTTAGAAAAATAAACAAATAGGGGTTC |
| | CGCGCACATTTCCCCGAAAAGTGCCACCTGACGCGCCCTGTAGCGGCGC |
| | ATTAAGCGCGGCGGGTGTGGTGGTTACGCGCAGCGTGACCGCTACACTT |
| | GCCAGCGCCCTAGCGCCCGCTCCTTTCGCTTTCTTCCCTTCCTTTCTCGCC |
| | ACGTTCGCCGGCTTTCCCCGTCAAGCTCTAAATCGGGGGCTCCCTTTAGG |
| | GTTCCGATTTAGTGCTTTACGGCACCTCGACCCCAAAAAACTTGATTAGG |
| | GTGATGGTTCACGTAGTGGGCCATCGCCCTGATAGACGGTTTTTCGCCCT |
| | TTGACGTTGGAGTCCACGTTCTTTAATAGTGGACTCTTGTTCCAAACTGG |
| | AACAACACTCAACCCTATCTCGGTCTATTCTTTTGATTTATAAGGGATTT |
| | TGCCGATTTCGGCCTATTGGTTAAAAAATGAGCTGATTTAACAAAAATTT |
| | AACGCGAATTTTAACAAAATATTAACGCTTACAATTTCCATTCGCCATTC |
| | AGGCTGCGCAACTGTTGGGAAGGGCGATCGGTGCGGGCCTCTTCGCTAT |
| | TACGCCAGCTGCGGCCGC |

What is claimed is:

1. A method for improving the efficiency and accuracy of gene knock-in using the non-retained end of Cpf1, comprising: using single or paired Cpf1 to generate free target DNA ends and free donor DNA ends, and combining with the complementary 5′-cohesive ends produced by Cpf1 to achieve more efficient and more accurate gene knock-in based on c-NHEJ; if it is needed to improve the efficiency and accuracy of knocking the target gene into one end of the recipient genome, the end of the recipient genome target that requires high ligation accuracy after cleavage shall be a free end, and at least ensure that the end of the donor target that needs to be correspondingly ligated after cleavage is a free end, with the free end of the donor complementarily ligated to the free end of the recipient genome requiring high accuracy; if it is necessary to improve the efficiency and accuracy of knocking the target gene into both ends of the recipient genome, both ends of the donor target after cleavage and the corresponding two ends of the recipient genome after cleavage shall be complementary free ends, and in this case, paired Cpf1 are required for both donor and recipient genome cleavage.

2. The method for improving the efficiency and accuracy of gene knock-in using the non-retained end of Cpf1 according to claim 1, wherein the method for improving the efficiency and accuracy of N-terminal target gene knock-in is as follows: the PAM of recipient Cpf1 target gene is located on W strand; the upstream Cpf1 target PAM of the donor DNA precursor is located on either W strand or C strand, while the downstream Cpf1 target PAM of the donor DNA precursor is located on C strand, and the corresponding 5′-cohesive ends of the recipient gene and the donor upon Cpf1 cleavage are completely complementary.

3. The method for improving the efficiency and accuracy of gene knock-in using the non-retained end of Cpf1 according to claim 1, wherein the method for improving the efficiency and accuracy of C-terminal target gene knock-in is as follows: the PAM of recipient Cpf1 target gene is located on C strand; the upstream Cpf1 target PAM of the donor DNA precursor is located on W strand, while the downstream Cpf1 target PAM of the donor DNA precursor is located on either C strand or W strand, and the corresponding 5′-cohesive ends of the recipient gene and the donor upon Cpf1 cleavage are completely complementary.

4. The method for improving the efficiency and accuracy of gene knock-in using the non-retained end of Cpf1 according to claim 1, wherein the method for improving the efficiency and accuracy of N-terminal or C-terminal knock-in of a DNA fragment or gene tag using Cpf1 non-retained end is carried out according to the following steps:
(1) based on the requirement for N-terminal or C-terminal tag knock-in, selecting a testable Cpf1 target in the targeted gene according to the strand where the Cpf1 PAM is located; since N-terminal tag knock-in requires precise ligation between the inserted tag and the junction of the downstream target gene, the downstream end of the two ends of the DSB generated by Cpf1 target cleavage should be a free PAM-distal end, i.e., the PAM of the Cpf1 target in the target gene is located on Watson strand; if it is C-terminal tag knock-in, precise ligation between the inserted tag and the junction of the upstream target gene is required, therefore the DSB generated by Cpf1 target cleavage should be a free PAM-distal end, i.e., the PAM of the Cpf1 target in the target gene is located on Crick strand;
(2) after selecting the Cpf1 target in the target gene, using T7E1 assay to test the cleavage efficiency of the selected target gene Cpf1 target in the target cells, and selecting the Cpf1 target that can be cleaved with high efficiency;
(3) designing the donor DNA for N-terminal knock-in: both sides of the donor DNA precursor should contain a Cpf1 target designed based on the target sequence of the target gene, the PAM of the upstream target can be located on either Watson strand or Crick strand, while the PAM of the downstream target must be located on Crick strand, so that paired Cpf1-sgRNA cleaves the donor DNA precursor to generate donor DNA with two 5′-cohesive ends, each of which is completely complementary to the corresponding end of the recipient gene, and the downstream end of the cleaved donor DNA is a free PAM-distal end;
(4) designing the donor DNA for C-terminal knock-in: both sides of the donor DNA precursor should contain a Cpf1 target designed based on the target sequence of the target gene, the PAM of the upstream target must be located on Watson strand, while the PAM of the downstream target can be located on either Watson strand or Crick strand, so that paired Cpf1-sgRNA cleaves the donor DNA precursor to generate donor DNA with two 5′-cohesive ends, each of which is completely complementary to the corresponding end of the recipient gene, and the upstream end of the cleaved donor DNA is a free PAM-distal end.

5. The method for improving the efficiency and accuracy of gene knock-in using the non-retained end of Cpf1 as according to claim 1, wherein the method for improving the efficiency and accuracy of knocking in a DNA fragment or gene using Cpf1 non-retained end is carried out according to the following steps:
(1) based on the requirement for the genomic target of DNA fragment or gene knock-in, identifying two adjacent testable Cpf1 genomic targets; the PAM of the upstream target shall be located on Crick strand, and the PAM of the downstream target shall be located on Watson strand;
(2) using target-specific PCR amplification to test the efficiency of Cpf1 in simultaneously cleaving the paired Cpf1 targets in the target cells, and selecting the paired targets that can be cleaved simultaneously with high efficiency;
(3) designing the donor DNA precursor for DNA fragment or gene knock-in: both sides of the donor DNA precursor contain two Cpf1 targets, the PAM of the upstream Cpf1 target is located on Watson strand, while the downstream PAM is located on Crick strand; paired Cpf1-sgRNA cleaves the donor DNA precursor to generate donor DNA with two 5′-cohesive ends, both of which are free PAM-distal ends; the upstream 5′-cohesive end shall be completely complementary to the upstream free end of the genomic Cpf1 target, and the downstream 5′-cohesive end shall be completely complementary to the downstream free end of the genomic Cpf1 target.


SEQUENCE LISTING (300 sequences)

SEQ ID NO: 1 - DNA, 734 bp, synthetic construct
  source 1..734: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgtagggataacagggtaatcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 2 - DNA, 1567 bp, synthetic construct
  source 1..1567: mol_type = other DNA
gggggtaggggaggcgcttttccaaggcagtctgagcatgcgcttagcagccccgctggcacttggcgctacacaagtggcctytggcctcgcacacattccacatccaccggtaggcgccaaccggctccgttctttggtggccccttcgcgccaccttctwctcctcccctagtcaggaagttcccccccgccccgcagctcgcgtcgtsaggacgtgacaaatggaagtagcacgtctcactagtctcgtcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcaggtcctcgccatggatcctgctagcatggtgagcaagggcgaggagaataacatggccgtcatcaaggagttcatgcgcttcaaggtgcgcatggagggctccgtgaacggccacgagttcgagatcgagggcgagggcgagggccgcccctacgagggcacccagaccgccaagctgaaggtgaccgagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagatgaggctgaagctgaaggacggtggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcggcatggacgagctgtacaagagatctcgagctcaagcttcgaattctgcagtcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttacgcgcccctatagtgagtcgtattaaaaa

SEQ ID NO: 3 - DNA, 3869 bp, synthetic construct
  source 1..3869: mol_type = other DNA
tttcaagatcagccagactgagttaccctcagtctggctgatcttgggaatatggtgaacttttattattttttaaatgtgctaattataatgaatttcttttcaatcaataggaggatgaaattttgaacagatcaccaagaaacagaaagccacgaagagagggcggtggcggtagtggtggcggcggcagcggtggcggtggtagcatggtgagcaagggcgaggagaataacatggccgtcatcaaggagttcatgcgcttcaaggtgcgcatggagggctccgtgaacggccacgagttcgagatcgagggcgagggcgagggccgcccctacgagggcacccagaccgccaagctgaaggtgaccgagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagatgaggctgaagctgaaggacggtggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcggcatggacgagctgtacaagagatctcgagctcaagcttcgaattctgcagtcgacggtaccgcgggcccgggatccaccggatctagataaaacaatcttaagagcttgatctgtgataccttctccctcctcccctgcaagaggcaggggaggagggagaagaaagatatcgaattctacccttctgaggcggaaagaaccagctggggctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctgcggccgc

SEQ ID NO: 4 - DNA, 3867 bp, synthetic construct
  source 1..3867: mol_type = other DNA
tttatactctacaactgaaagatcctgtctttcagttgtagagtataagattgcggatatgggacacttaaaatactacttggctcccaagatcgaggatgaagaaggatctggcggtggcggtagtggtggcggcggcagcggtggcggtggtagcatggtgagcaagggcgaggagaataacatggccgtcatcaaggagttcatgcgcttcaaggtgcgcatggagggctccgtgaacggccacgagttcgagatcgagggcgagggcgagggccgcccctacgagggcacccagaccgccaagctgaaggtgaccgagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagatgaggctgaagctgaaggacggtggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcggcatggacgagctgtacaagagatctcgagctcaagcttcgaattctgcagtcgacggtaccgcgggcccgggatccaccggatctagataagcattcttaaaattcaagaaaataaaactaagctctttgagaactgcttctaagatgccagcatatactgaagtcttccctgtcaccaaatttgtacctctaaggtacaaatttggtgacagaaagatatcgaattctacccttctgaggcggaaagaaccagctggggctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctgcggccgc

SEQ ID NO: 5 - DNA, 20 bp, synthetic construct
  source 1..20: mol_type = other DNA
tggctggcgtggaaatattc

SEQ ID NO: 6 - DNA, 20 bp, synthetic construct
  source 1..20: mol_type = other DNA
ccggtttggactcagagtat

SEQ ID NO: 7 - DNA, 20 bp, synthetic construct
  source 1..20: mol_type = other DNA
caggcagaagtatgcaaagc

SEQ ID NO: 8 - DNA, 20 bp, synthetic construct
  source 1..20: mol_type = other DNA
cctcggcctctgcataaata

SEQ ID NO: 9 - DNA, 19 bp, synthetic construct
  source 1..19: mol_type = other DNA
tctggagcatgcgctttag

SEQ ID NO: 10 - DNA, 20 bp, synthetic construct
  source 1..20: mol_type = other DNA
taccggtggatgtggaatgt

SEQ ID NO: 11 - DNA, 18 bp, synthetic construct
  source 1..18: mol_type = other DNA
ctggacggcgacgtaaac

SEQ ID NO: 12 - DNA, 19 bp, synthetic construct
  source 1..19: mol_type = other DNA
cggtggtgcagatgaactt

SEQ ID NO: 13 - DNA, 17 bp, synthetic construct
  source 1..17: mol_type = other DNA
agctcgccgaccactac

SEQ ID NO: 14 - DNA, 19 bp, synthetic construct
  source 1..19: mol_type = other DNA
tccagcaggaccatgtgat

SEQ ID NO: 15 - DNA, 21 bp, synthetic construct
  source 1..21: mol_type = other DNA
ggacaagacttcccacagatt

SEQ ID NO: 16 - DNA, 21 bp, synthetic construct
  source 1..21: mol_type = other DNA
ggcggatcacaagcaataata

SEQ ID NO: 17 - DNA, 21 bp, synthetic construct
  source 1..21: mol_type = other DNA
tccacatttgggcctattctc

SEQ ID NO: 18 - DNA, 21 bp, synthetic construct
  source 1..21: mol_type = other DNA
agacatccacctgaaaccatt

SEQ ID NO: 19 - DNA, 21 bp, synthetic construct
  source 1..21: mol_type = other DNA
ggggctctgagatttcataaa

SEQ ID NO: 20 - DNA, 20 bp, synthetic construct
  source 1..20: mol_type = other DNA
cccaaatctcatcagaaagg

SEQ ID NO: 21 - DNA, 20 bp, synthetic construct
  source 1..20: mol_type = other DNA
tggattaccctgttatccct

SEQ ID NO: 22 - DNA, 20 bp, synthetic construct
  source 1..20: mol_type = other DNA
cttgctgatcatgtgaagga

SEQ ID NO: 23 - DNA, 20 bp, synthetic construct
  source 1..20: mol_type = other DNA
ttgagctcgagatctgagtc

SEQ ID NO: 24 - DNA, 20 bp, synthetic construct
  source 1..20: mol_type = other DNA
cttccggctcgtatgttgtg

SEQ ID NO: 25 - DNA, 20 bp, synthetic construct
  source 1..20: mol_type = other DNA
tgtgagttagctcactcatt

SEQ ID NO: 26 - DNA, 20 bp, synthetic construct
  source 1..20: mol_type = other DNA
attccacacaacatacgagc

SEQ ID NO: 27 - DNA, 20 bp, synthetic construct
  source 1..20: mol_type = other DNA
ggataacagggtaatcaagg

SEQ ID NO: 28 - DNA, 20 bp, synthetic construct
  source 1..20: mol_type = other DNA
agggcatcgtagggataaca

SEQ ID NO: 29 - DNA, 20 bp, synthetic construct
  source 1..20: mol_type = other DNA
tgtcccctccaccccacagt

SEQ ID NO: 30 - DNA, 20 bp, synthetic construct
  source 1..20: mol_type = other DNA
agacccaatatcaggagact

SEQ ID NO: 31 - DNA, 20 bp, synthetic construct
  source 1..20: mol_type = other DNA
tggtatcaaggttacaagac

SEQ ID NO: 32 - DNA, 20 bp, synthetic construct
  source 1..20: mol_type = other DNA
cacgttcaccttgccccaca

SEQ ID NO: 33 - DNA, 20 bp, synthetic construct
  source 1..20: mol_type = other DNA
catggtgcatctgactcctg

SEQ ID NO: 34 - DNA, 20 bp, synthetic construct
  source 1..20: mol_type = other DNA
cctaagaatgagaaaggcaa

SEQ ID NO: 35 - DNA, 20 bp, synthetic construct
  source 1..20: mol_type = other DNA
gatgctgcagcccgccctgc

SEQ ID NO: 36 - DNA, 23 bp, synthetic construct
  source 1..23: mol_type = other DNA
acccactccccatttcaaccaaa

SEQ ID NO: 37 - DNA, 23 bp, synthetic construct
  source 1..23: mol_type = other DNA
tgtaaatctgaagctttcccaaa

SEQ ID NO: 38 - DNA, 23 bp, synthetic construct
  source 1..23: mol_type = other DNA
ctgattagaattctacccctctt

SEQ ID NO: 39 - DNA, 23 bp, synthetic construct
  source 1..23: mol_type = other DNA
cagattgtgttgccattattagt

SEQ ID NO: 40 - DNA, 23 bp, synthetic construct
  source 1..23: mol_type = other DNA
tgataaagtgaaaagagcactaa

SEQ ID NO: 41 - DNA, 23 bp, synthetic construct
  source 1..23: mol_type = other DNA
taagaaaagcattttaaatataa

SEQ ID NO: 42 - DNA, 23 bp, synthetic construct
  source 1..23: mol_type = other DNA
tagcatatagtataataaaaaag

SEQ ID NO: 43 - DNA, 23 bp, synthetic construct
  source 1..23: mol_type = other DNA
atcaggcaagtgtgggtccattt

SEQ ID NO: 44 - DNA, 23 bp, synthetic construct
  source 1..23: mol_type = other DNA
caagaatctatgtctctaaaatg

SEQ ID NO: 45 - DNA, 23 bp, synthetic construct
  source 1..23: mol_type = other DNA
ctcagttcctaccaggacagctg

SEQ ID NO: 46 - DNA, 23 bp, synthetic construct
  source 1..23: mol_type = other DNA
gagaagagggagagaaaacagct

SEQ ID NO: 47 - DNA, 23 bp, synthetic construct
  source 1..23: mol_type = other DNA
acccttgccttgcagcaaagaaa

SEQ ID NO: 48 - DNA, 23 bp, synthetic construct
  source 1..23: mol_type = other DNA
atgggggattacactgaatcttt

SEQ ID NO: 49 - DNA, 23 bp, synthetic construct
  source 1..23: mol_type = other DNA
acaaatcacaaaaccaacatttg

SEQ ID NO: 50 - DNA, 23 bp, synthetic construct
  source 1..23: mol_type = other DNA
gaaccaagggtcttagagtttta

SEQ ID NO: 51 - DNA, 23 bp, synthetic construct
  source 1..23: mol_type = other DNA
acattaagtacaaatgtttaaca

SEQ ID NO: 52 - DNA, 20 bp, synthetic construct
  source 1..20: mol_type = other DNA
ccgcgctgttctcctcttcc

SEQ ID NO: 53 - DNA, 20 bp, synthetic construct
  source 1..20: mol_type = other DNA
atcgccctcgccctcgccgg

SEQ ID NO: 54 - DNA, 20 bp, synthetic construct
  source 1..20: mol_type = other DNA
tcccgaggcccggcattctg

SEQ ID NO: 55 - DNA, 20 bp, synthetic construct
  source 1..20: mol_type = other DNA
gccgtccagctcgaccagga

SEQ ID NO: 56 - DNA, 20 bp, synthetic construct
  source 1..20: mol_type = other DNA
gggctcgatcctctagttgg

SEQ ID NO: 57 - DNA, 20 bp, synthetic construct
  source 1..20: mol_type = other DNA
cacgacaggtttcccgactg

SEQ ID NO: 58 - DNA, 20 bp, synthetic construct
  source 1..20: mol_type = other DNA
tccgccatgcccgaaggcta

SEQ ID NO: 59 - DNA, 20 bp, synthetic construct
  source 1..20: mol_type = other DNA
tccctcgatgttgtggcgga

SEQ ID NO: 60 - DNA, 20 bp, synthetic construct
  source 1..20: mol_type = other DNA
cgaaggctacgtccaggagc

SEQ ID NO: 61 - DNA, 20 bp, synthetic construct
  source 1..20: mol_type = other DNA
tgtggcggatcttgaagttc

SEQ ID NO: 62 - DNA, 20 bp, synthetic construct
  source 1..20: mol_type = other DNA
tgggaccaccttatattccc

SEQ ID NO: 63 - DNA, 20 bp, synthetic construct
  source 1..20: mol_type = other DNA
catcgtaagcaaaccttaga

SEQ ID NO: 64 - DNA, 20 bp, synthetic construct
  source 1..20: mol_type = other DNA
gggtgggaaaatagaccaat

SEQ ID NO: 65 - DNA, 20 bp, synthetic construct
  source 1..20: mol_type = other DNA
cagagccatctattgcttac

SEQ ID NO: 66 - DNA, 20 bp, synthetic construct
  source 1..20: mol_type = other DNA
gctggtgaagacgttacaca

SEQ ID NO: 67 - DNA, 20 bp, synthetic construct
  source 1..20: mol_type = other DNA
aagccaagatcctaggtaaa

SEQ ID NO: 68 - DNA, 19 bp, synthetic construct
  source 1..19: mol_type = other DNA
gccaggctgaaaggatagg

SEQ ID NO: 69 - DNA, 20 bp, synthetic construct
  source 1..20: mol_type = other DNA
agaaaaagcactggcgaatc

SEQ ID NO: 70 - DNA, 20 bp, synthetic construct
  source 1..20: mol_type = other DNA
ccctcttgcaaaagtgtcca

SEQ ID NO: 71 - DNA, 20 bp, synthetic construct
  source 1..20: mol_type = other DNA
tttacgtctgcctcctcacc

SEQ ID NO: 72 - DNA, 20 bp, synthetic construct
  source 1..20: mol_type = other DNA
ctggcgtttggactcagttc

SEQ ID NO: 73 - DNA, 20 bp, synthetic construct
  source 1..20: mol_type = other DNA
cgctggtctcaagtctcttg

SEQ ID NO: 74 - DNA, 21 bp, synthetic construct
  source 1..21: mol_type = other DNA
tggatcatgtagttgcagggt

SEQ ID NO: 75 - DNA, 20 bp, synthetic construct
  source 1..20: mol_type = other DNA
ccaaagaaggcgggaaagaa

SEQ ID NO: 76 - DNA, 19 bp, synthetic construct
  source 1..19: mol_type = other DNA
cagctgaccactgtgatgc

SEQ ID NO: 77 - DNA, 27 bp, synthetic construct
  source 1..27: mol_type = other DNA
atcactgataggtataaagtcaaaacc

SEQ ID NO: 78 - DNA, 20 bp, synthetic construct
  source 1..20: mol_type = other DNA
ttagctatggacaggagggg

SEQ ID NO: 79 - DNA, 20 bp, synthetic construct
  source 1..20: mol_type = other DNA
aatatgttgtgtgcgccctc

SEQ ID NO: 80 - DNA, 20 bp, synthetic construct
  source 1..20: mol_type = other DNA
ggcagagctggtgaatgaac

SEQ ID NO: 81 - DNA, 20 bp, synthetic construct
  source 1..20: mol_type = other DNA
tgcgacttctctgttcgtct

SEQ ID NO: 82 - DNA, 23 bp, synthetic construct
  source 1..23: mol_type = other DNA
gcaataaccttcacctctcgtaa

SEQ ID NO: 83 - DNA, 27 bp, synthetic construct
  source 1..27: mol_type = other DNA
ggggtctatagatttagttgaattttc

SEQ ID NO: 84 - DNA, 20 bp, synthetic construct
  source 1..20: mol_type = other DNA
agcatttcaggatgggtctt

SEQ ID NO: 85 - DNA, 27 bp, synthetic construct
  source 1..27: mol_type = other DNA
tcattcaaaataacattgaaaagaaca

SEQ ID NO: 86 - DNA, 20 bp, synthetic construct
  source 1..20: mol_type = other DNA
caaggcagacaaccaagaaa

SEQ ID NO: 87 - DNA, 20 bp, synthetic construct
  source 1..20: mol_type = other DNA
gggaaacccaaagaagtgct

SEQ ID NO: 88 - DNA, 24 bp, synthetic construct
  source 1..24: mol_type = other DNA
tagttatgaggagtgaggtggact

SEQ ID NO: 89 - DNA, 27 bp, synthetic construct
  source 1..27: mol_type = other DNA
aagcaaattaacattaaaagtcagaaa

SEQ ID NO: 90 - DNA, 20 bp, synthetic construct
  source 1..20: mol_type = other DNA
ggtttccctgcactatcctg

SEQ ID NO: 91 - DNA, 20 bp, synthetic construct
  source 1..20: mol_type = other DNA
ttgcctgcaaaccacaatta

SEQ ID NO: 92 - DNA, 23 bp, synthetic construct
  source 1..23: mol_type = other DNA
ctgtccctgagccccccacctcc

SEQ ID NO: 93 - DNA, 23 bp, synthetic construct
  source 1..23: mol_type = other DNA
tacccttttcaggagaggcctcc

SEQ ID NO: 94 - DNA, 23 bp, synthetic construct
  source 1..23: mol_type = other DNA
agtaagcagtaatcaataccatg

SEQ ID NO: 95 - DNA, 23 bp, synthetic construct
  source 1..23: mol_type = other DNA
tcttggactggcttgactcatgg

SEQ ID NO: 96 - DNA, 23 bp, synthetic construct
  source 1..23: mol_type = other DNA
attcaagttttcccccatcaaat

SEQ ID NO: 97 - DNA, 23 bp, synthetic construct
  source 1..23: mol_type = other DNA
tcttatacttaacttttttttta

SEQ ID NO: 98 - DNA, 23 bp, synthetic construct
  source 1..23: mol_type = other DNA
taatcttacctattcctaaaatt

SEQ ID NO: 99 - DNA, 23 bp, synthetic construct
  source 1..23: mol_type = other DNA
tagagaagccatttctcaaaatt

SEQ ID NO: 100 - DNA, 23 bp, synthetic construct
  source 1..23: mol_type = other DNA
atatatcaaggcaaaacatgtta

SEQ ID NO: 101 - DNA, 23 bp, synthetic construct
  source 1..23: mol_type = other DNA
gatctccttttgacaacaatagc

SEQ ID NO: 102 - DNA, 23 bp, synthetic construct
  source 1..23: mol_type = other DNA
gagacagggtttctctgtatagc

SEQ ID NO: 103 - DNA, 23 bp, synthetic construct
  source 1..23: mol_type = other DNA
aatgagtgtcagattgttttgaa

SEQ ID NO: 104 - DNA, 23 bp, synthetic construct
  source 1..23: mol_type = other DNA
taagctattacactttccttcaa

SEQ ID NO: 105 - DNA, 23 bp, synthetic construct
  source 1..23: mol_type = other DNA
aaaattttaggaataggtaagat

SEQ ID NO: 106 - DNA, 23 bp, synthetic construct
  source 1..23: mol_type = other DNA
agaaatggcttctctagaaagat

SEQ ID NO: 107 - DNA, 23 bp, synthetic construct
  source 1..23: mol_type = other DNA
acataactaaaacaagcacagtg

SEQ ID NO: 108 - DNA, 23 bp, synthetic construct
  source 1..23: mol_type = other DNA
gttcacaccacaaatgaacagtg

SEQ ID NO: 109 - DNA, 23 bp, synthetic construct
  source 1..23: mol_type = other DNA
taagcttcatccatttgtaacat

SEQ ID NO: 110 - DNA, 23 bp, synthetic construct
  source 1..23: mol_type = other DNA
accattagggcaaatggcaacat

SEQ ID NO: 111 - DNA, 23 bp, synthetic construct
  source 1..23: mol_type = other DNA
atcccagcactaaggaagcagag

SEQ ID NO: 112 - DNA, 23 bp, synthetic construct
  source 1..23: mol_type = other DNA
gttttcaagacagggtttctctg

SEQ ID NO: 113 - DNA, 23 bp, synthetic construct
  source 1..23: mol_type = other DNA
tcaccagcaggaccggggggacc

SEQ ID NO: 114 - DNA, 23 bp, synthetic construct
  source 1..23: mol_type = other DNA
cccccttctttgccaacgggacc

SEQ ID NO: 115 - DNA, 23 bp, synthetic construct
  source 1..23: mol_type = other DNA
ctattatacttaatgccttaaca

SEQ ID NO: 116 - DNA, 23 bp, synthetic construct
  source 1..23: mol_type = other DNA
ccattctaaactgtaccctgtta

SEQ ID NO: 117 - DNA, 23 bp, synthetic construct
  source 1..23: mol_type = other DNA
atattgctaatagcagctacaat

SEQ ID NO: 118 - DNA, 23 bp, synthetic construct
  source 1..23: mol_type = other DNA
tttcagggcaataatgatacaat

SEQ ID NO: 119 - DNA, 23 bp, synthetic construct
  source 1..23: mol_type = other DNA
tctcccaaccccctcccttcatt

SEQ ID NO: 120 - DNA, 23 bp, synthetic construct
  source 1..23: mol_type = other DNA
tcattttactatattttatcatt

SEQ ID NO: 121 - DNA, 23 bp, synthetic construct
  source 1..23: mol_type = other DNA
cttcctcacaatcttgctatttt

SEQ ID NO: 122 - DNA, 23 bp, synthetic construct
  source 1..23: mol_type = other DNA
tcatttaatgcttctaaaatttt

SEQ ID NO: 123 - DNA, 23 bp, synthetic construct
  source 1..23: mol_type = other DNA
actattctgggtacctcacagac

SEQ ID NO: 124 - DNA, 23 bp, synthetic construct
  source 1..23: mol_type = other DNA
accacgttgcccaagctagtctg

SEQ ID NO: 125 - DNA, 23 bp, synthetic construct
  source 1..23: mol_type = other DNA
ttttttgttttgagacacagttt

SEQ ID NO: 126 - DNA, 23 bp, synthetic construct
  source 1..23: mol_type = other DNA
tatttttagtagagacagagttt

SEQ ID NO: 127 - DNA, 23 bp, synthetic construct
  source 1..23: mol_type = other DNA
aatgcacctctcatagtaactga

SEQ ID NO: 128 - DNA, 23 bp, synthetic construct
  source 1..23: mol_type = other DNA
tttcttcttatagttctgtcagt

SEQ ID NO: 129 - DNA, 23 bp, synthetic construct
  source 1..23: mol_type = other DNA
tctcagtcttttgacttgtcttt

SEQ ID NO: 130 - DNA, 23 bp, synthetic construct
  source 1..23: mol_type = other DNA
ctattcagcctgacttcatcttt

SEQ ID NO: 131 - DNA, 23 bp, synthetic construct
  source 1..23: mol_type = other DNA
accttccgctcccctttcctcct

SEQ ID NO: 132 - DNA, 23 bp, synthetic construct
  source 1..23: mol_type = other DNA
ttcggcggaccttaccctctcct

SEQ ID NO: 133 - DNA, 23 bp, synthetic construct
  source 1..23: mol_type = other DNA
cctcctggcagtgttttaaaatt

SEQ ID NO: 134 - DNA, 23 bp, synthetic construct
  source 1..23: mol_type = other DNA
ttacagttctgcaattaaaattt

SEQ ID NO: 135 - DNA, 23 bp, synthetic construct
  source 1..23: mol_type = other DNA
caggtctggcccaggtacctctt

SEQ ID NO: 136 - DNA, 23 bp, synthetic construct
  source 1..23: mol_type = other DNA
tgcaaattcttactttgaaaatg

SEQ ID NO: 137 - DNA, 23 bp, synthetic construct
  source 1..23: mol_type = other DNA
aagcattgacgcacagacaaatg

SEQ ID NO: 138 - DNA, 23 bp, synthetic construct
  source 1..23: mol_type = other DNA
taaactatatattctatattcaa

SEQ ID NO: 139 - DNA, 23 bp, synthetic construct
  source 1..23: mol_type = other DNA
taactgtttaataaactattcaa

SEQ ID NO: 140 - DNA, 23 bp, synthetic construct
  source 1..23: mol_type = other DNA
aaaagtcttttggtgttacctgg

SEQ ID NO: 141 - DNA, 23 bp, synthetic construct
  source 1..23: mol_type = other DNA
tttgggaagctggatgagcctgg

SEQ ID NO: 142 - DNA, 23 bp, synthetic construct
  source 1..23: mol_type = other DNA
agtttatttcacaggacaagagt

SEQ ID NO: 143 - DNA, 23 bp, synthetic construct
  source 1..23: mol_type = other DNA
gtaatcaaagagcccttaactct

SEQ ID NO: 144 - DNA, 23 bp, synthetic construct
  source 1..23: mol_type = other DNA
tgtattatcgatggctgctttct

SEQ ID NO: 145 - DNA, 23 bp, synthetic construct
  source 1..23: mol_type = other DNA
gatactctaaagtagagtagaaa

SEQ ID NO: 146 - DNA, 23 bp, synthetic construct
  source 1..23: mol_type = other DNA
ttcctgtctgcagagctgtgaaa

SEQ ID NO: 147 - DNA, 23 bp, synthetic construct
  source 1..23: mol_type = other DNA
tttgctgcaaggcaagggtgaaa

SEQ ID NO: 148 - DNA, 23 bp, synthetic construct
  source 1..23: mol_type = other DNA
ctttctctttaccatgctgtgac

SEQ ID NO: 149 - DNA, 23 bp, synthetic construct
  source 1..23: mol_type = other DNA
tcaaaactactcaactctgtcac

SEQ ID NO: 150 - DNA, 23 bp, synthetic construct
  source 1..23: mol_type = other DNA
acagctctgcagacaggaagaaa

SEQ ID NO: 151 - DNA, 23 bp, synthetic construct
  source 1..23: mol_type = other DNA
cttgtacatattttgttcttttc

SEQ ID NO: 152 - DNA, 23 bp, synthetic construct
  source 1..23: mol_type = other DNA
tttcaggattctttagagttttc

SEQ ID NO: 153 - DNA, 23 bp, synthetic construct
  source 1..23: mol_type = other DNA
ggtgtcttttatatgttgttttc

SEQ ID NO: 154 - DNA, 23 bp, synthetic construct
  source 1..23: mol_type = other DNA
ataaatatttatcttgaatgcct

SEQ ID NO: 155 - DNA, 23 bp, synthetic construct
  source 1..23: mol_type = other DNA
cagcatctaaaacatgtcaggca

SEQ ID NO: 156 - DNA, 23 bp, synthetic construct
  source 1..23: mol_type = other DNA
tggttttcagtatatgagtcttt

SEQ ID NO: 157 - DNA, 23 bp, synthetic construct
  source 1..23: mol_type = other DNA
attgtcttaatctaggaagctcc

SEQ ID NO: 158 - DNA, 23 bp, synthetic construct
  source 1..23: mol_type = other DNA
cgctccccatcaagctgggctcc

SEQ ID NO: 159 - DNA, 23 bp, synthetic construct
  source 1..23: mol_type = other DNA
aaatatctctatcagctattaac

SEQ ID NO: 160 - DNA, 23 bp, synthetic construct
  source 1..23: mol_type = other DNA
aaatatgcatgtggtaaagttaa

SEQ ID NO: 161 - DNA, 23 bp, synthetic construct
  source 1..23: mol_type = other DNA
caaaggtaactacaacaatctct

SEQ ID NO: 162 - DNA, 23 bp, synthetic construct
  source 1..23: mol_type = other DNA
ccaatggagatactgccaagaga

SEQ ID NO: 163 - DNA, 23 bp, synthetic construct
  source 1..23: mol_type = other DNA
catgtatgttctcacttaatcct

SEQ ID NO: 164 - DNA, 23 bp, synthetic construct
  source 1..23: mol_type = other DNA
ttccttatttggaaaataaggat

SEQ ID NO: 165 - DNA, 23 bp, synthetic construct
  source 1..23: mol_type = other DNA
tgcctttgatctcagagggtctg

SEQ ID NO: 166 - DNA, 23 bp, synthetic construct
  source 1..23: mol_type = other DNA
caagttattaatatacgcgtctg

SEQ ID NO: 167 - DNA, 23 bp, synthetic construct
  source 1..23: mol_type = other DNA
agtggagagttggtatatttata

SEQ ID NO: 168 - DNA, 23 bp, synthetic construct
  source 1..23: mol_type = other DNA
tccttgtttccctgtacttaatt

SEQ ID NO: 169 - DNA, 23 bp, synthetic construct
  source 1..23: mol_type = other DNA
tctcattattggagaatataatt

SEQ ID NO: 170 - DNA, 23 bp, synthetic construct
  source 1..23: mol_type = other DNA
ttcctttttattgagtattgaat

SEQ ID NO: 171 - DNA, 23 bp, synthetic construct
  source 1..23: mol_type = other DNA
ctaagcaaaaaaagctagattca

SEQ ID NO: 172 - DNA, 23 bp, synthetic construct
  source 1..23: mol_type = other DNA
aatcttgaacacacatgctattt

SEQ ID NO: 173 - DNA, 23 bp, synthetic construct
  source 1..23: mol_type = other DNA
atttggcttgccaaggtctattt

SEQ ID NO: 174 - DNA, 23 bp, synthetic construct
  source 1..23: mol_type = other DNA
ttctctgcctactctctctcttt

SEQ ID NO: 175 - DNA, 23 bp, synthetic construct
  source 1..23: mol_type = other DNA
ccctggtcactgctgtcctcttt

SEQ ID NO: 176 - DNA, 23 bp, synthetic construct
  source 1..23: mol_type = other DNA
ccagaaaggggtgtgaatctcta

SEQ ID NO: 177 - DNA, 23 bp, synthetic construct
  source 1..23: mol_type = other DNA
acctccagtcttctgagttagag

SEQ ID NO: 178 - DNA, 23 bp, synthetic construct
  source 1..23: mol_type = other DNA
aaaatatgcaacagtgttctttt

SEQ ID NO: 179 - DNA, 23 bp, synthetic construct
  source 1..23: mol_type = other DNA
ttgcttcttggaatctgatcttg

SEQ ID NO: 180 - DNA, 23 bp, synthetic construct
  source 1..23: mol_type = other DNA
actcagtcaaaaattccacaaga

SEQ ID NO: 181 - DNA, 23 bp, synthetic construct
  source 1..23: mol_type = other DNA
ttgctgctagatattccttcaca

SEQ ID NO: 182 - DNA, 23 bp, synthetic construct
  source 1..23: mol_type = other DNA
tctctatttacttcctcgtcaca

SEQ ID NO: 183 - DNA, 23 bp, synthetic construct
  source 1..23: mol_type = other DNA
tctaggtaaatattttagttggc

SEQ ID NO: 184 - DNA, 23 bp, synthetic construct
  source 1..23: mol_type = other DNA
ttataaaatttaatcttagccaa

SEQ ID NO: 185 - DNA, 20 bp, synthetic construct
  source 1..20: mol_type = other DNA
ggagaggcgttcaggaagat

SEQ ID NO: 186 - DNA, 20 bp, synthetic construct
  source 1..20: mol_type = other DNA
tctagtcgaccccactacct

SEQ ID NO: 187 - DNA, 22 bp, synthetic construct
  source 1..22: mol_type = other DNA
tcttacatattgccaggctgat

SEQ ID NO: 188 - DNA, 20 bp, synthetic construct
  source 1..20: mol_type = other DNA
gtcctgaagaagcttggcaa

SEQ ID NO: 189 - DNA, 23 bp, synthetic construct
  source 1..23: mol_type = other DNA
tgttctcactgagctacatcctg

SEQ ID NO: 190 - DNA, 20 bp, synthetic construct
  source 1..20: mol_type = other DNA
ctgtgacccacgtaaagcaa

SEQ ID NO: 191 - DNA, 22 bp, synthetic construct
  source 1..22: mol_type = other DNA
agcccttgttctttatcaccct

SEQ ID NO: 192 - DNA, 22 bp, synthetic construct
  source 1..22: mol_type = other DNA
aatatccaacttagccaggcgt

SEQ ID NO: 193 - DNA, 18 bp, synthetic construct
  source 1..18: mol_type = other DNA
tgtgttggtgcgagcaat

SEQ ID NO: 194 - DNA, 21 bp, synthetic construct
  source 1..21: mol_type = other DNA
tctgccagatattcagcaatg

SEQ ID NO: 195 - DNA, 21 bp, synthetic construct
  source 1..21: mol_type = other DNA
ggcttgacttgtcactgtgct

SEQ ID NO: 196 - DNA, 20 bp, synthetic construct
  source 1..20: mol_type = other DNA
gcccaattccaactgtgaag

SEQ ID NO: 197 - DNA, 21 bp, synthetic construct
  source 1..21: mol_type = other DNA
actcagtggttcttttgagca

SEQ ID NO: 198 - DNA, 20 bp, synthetic construct
  source 1..20: mol_type = other DNA
ccccactttttctttcacca

SEQ ID NO: 199 - DNA, 21 bp, synthetic construct
  source 1..21: mol_type = other DNA
agacaaggagagcaaatgtga

SEQ ID NO: 200 - DNA, 20 bp, synthetic construct
  source 1..20: mol_type = other DNA
accttgtttgccaggttcac

SEQ ID NO: 201 - DNA, 20 bp, synthetic construct
  source 1..20: mol_type = other DNA
ggcttgccactatgatgctt

SEQ ID NO: 202 - DNA, 23 bp, synthetic construct
  source 1..23: mol_type = other DNA
acacatacacaactctggaactc

SEQ ID NO: 203 - DNA, 26 bp, synthetic construct
  source 1..26: mol_type = other DNA
aagttacttaatgtatctcagagata

SEQ ID NO: 204 - DNA, 20 bp, synthetic construct
  source 1..20: mol_type = other DNA
atgggacgcttgatgttttc

SEQ ID NO: 205 - DNA, 20 bp, synthetic construct
  source 1..20: mol_type = other DNA
ggcctagcttggactcagaa

SEQ ID NO: 206 - DNA, 30 bp, synthetic construct
  source 1..30: mol_type = other DNA
tttttgtttatcttatttctaatactttcc

SEQ ID NO: 207 - DNA, 21 bp, synthetic construct
  source 1..21: mol_type = other DNA
tgcagaattagcaggtgagag

SEQ ID NO: 208 - DNA, 22 bp, synthetic construct
  source 1..22: mol_type = other DNA
ccaggagatgcttcacttttct

SEQ ID NO: 209 - DNA, 24 bp, synthetic construct
  source 1..24: mol_type = other DNA
tgaaaagtgaaaataagccagtca

SEQ ID NO: 210 - DNA, 20 bp, synthetic construct
  source 1..20: mol_type = other DNA
ctgcaatcccagcactttag

SEQ ID NO: 211 - DNA, 19 bp, synthetic construct
  source 1..19: mol_type = other DNA
tgaggtcgggagtttgagg

SEQ ID NO: 212 - DNA, 27 bp, synthetic construct
  source 1..27: mol_type = other DNA
agcataatgtcctcaagatacatctac

SEQ ID NO: 213 - DNA, 23 bp, synthetic construct
  source 1..23: mol_type = other DNA
ggtcaaccttgtaatcatgctgt

SEQ ID NO: 214 - DNA, 20 bp, synthetic construct
  source 1..20: mol_type = other DNA
gtggaggttgcagtgagcta

SEQ ID NO: 215 - DNA, 20 bp, synthetic construct
  source 1..20: mol_type = other DNA
tttgcaaaagccattttcct

SEQ ID NO: 216 - DNA, 20 bp, synthetic construct
  source 1..20: mol_type = other DNA
agaatttggaccagccacac

SEQ ID NO: 217 - DNA, 19 bp, synthetic construct
  source 1..19: mol_type = other DNA
gcggtctcccagctacctc

SEQ ID NO: 218 - DNA, 20 bp, synthetic construct
  source 1..20: mol_type = other DNA
agattaagtggggcagcaga

SEQ ID NO: 219 - DNA, 21 bp, synthetic construct
  source 1..21: mol_type = other DNA
tgcagggaaaaagcttacaaa

SEQ ID NO: 220 - DNA, 22 bp, synthetic construct
  source 1..22: mol_type = other DNA
tgttaatctgtgggtggtagga

SEQ ID NO: 221 - DNA, 23 bp, synthetic construct
  source 1..23: mol_type = other DNA
ccagttaaccagccttagtatgc

SEQ ID NO: 222 - DNA, 19 bp, synthetic construct
  source 1..19: mol_type = other DNA
gaggcagaggttgcactga

SEQ ID NO: 223 - DNA, 23 bp, synthetic construct
  source 1..23: mol_type = other DNA
agctcttcgtagtggtgcattta

SEQ ID NO: 224 - DNA, 23 bp, synthetic construct
  source 1..23: mol_type = other DNA
tccaatcacttcctctatccaga

SEQ ID NO: 225 - DNA, 20 bp, synthetic construct
  source 1..20: mol_type = other DNA
caggaggggacagatggatg

SEQ ID NO: 226 - DNA, 20 bp, synthetic construct
  source 1..20: mol_type = other DNA
catcccctccccttctttca

SEQ ID NO: 227 - DNA, 20 bp, synthetic construct
  source 1..20: mol_type = other DNA
ggtgcctgacgaataagctg

SEQ ID NO: 228 - DNA, 23 bp, synthetic construct
  source 1..23: mol_type = other DNA
aaatactgatgatccccactagg

SEQ ID NO: 229 - DNA, 19 bp, synthetic construct
  source 1..19: mol_type = other DNA
gaagtccgtgggccaaatc

SEQ ID NO: 230 - DNA, 24 bp, synthetic construct
  source 1..24: mol_type = other DNA
agcatggtaaagagaaaggaaagt

SEQ ID NO: 231 - DNA, 22 bp, synthetic construct
  source 1..22: mol_type = other DNA
agagttgctgttattctggtag

SEQ ID NO: 232 - DNA, 23 bp, synthetic construct
  source 1..23: mol_type = other DNA
agccattggtacctgtaagtaaa

SEQ ID NO: 233 - DNA, 24 bp, synthetic construct
  source 1..24: mol_type = other DNA
gcccgttcatttatgtattatcga

SEQ ID NO: 234 - DNA, 25 bp, synthetic construct
  source 1..25: mol_type = other DNA
tgagcatgactttggacaataattt

SEQ ID NO: 235 - DNA, 23 bp, synthetic construct
  source 1..23: mol_type = other DNA
tgagtcttttgcctccttgttta

SEQ ID NO: 236 - DNA, 27 bp, synthetic construct
  source 1..27: mol_type = other DNA
tggaaagtaaggagtcaaattatctct

SEQ ID NO: 237 - DNA, 23 bp, synthetic construct
  source 1..23: mol_type = other DNA
tgtagaaacacaactgctctttg

SEQ ID NO: 238 - DNA, 21 bp, synthetic construct
  source 1..21: mol_type = other DNA
agatcaggaacaagacaagga

SEQ ID NO: 239 - DNA, 20 bp, synthetic construct
  source 1..20: mol_type = other DNA
tgtgaggcgaaaatcccatt

SEQ ID NO: 240 - DNA, 20 bp, synthetic construct
  source 1..20: mol_type = other DNA
tgtgcccggcctacttattt

SEQ ID NO: 241 - DNA, 18 bp, synthetic construct
  source 1..18: mol_type = other DNA
ctgagaaggcggtggagg

SEQ ID NO: 242 - DNA, 20 bp, synthetic construct
  source 1..20: mol_type = other DNA
ctgagtgaggcagtgaggaa

SEQ ID NO: 243 - DNA, 23 bp, synthetic construct
  source 1..23: mol_type = other DNA
acaggggtcatcattgttaaaga

SEQ ID NO: 244 - DNA, 20 bp, synthetic construct
  source 1..20: mol_type = other DNA
ccagtgcctctctcctcaat

SEQ ID NO: 245 - DNA, 23 bp, synthetic construct
  source 1..23: mol_type = other DNA
ggggagatatattgtagcttctc

SEQ ID NO: 246 - DNA, 20 bp, synthetic construct
  source 1..20: mol_type = other DNA
tgtgcagtaatgaccaggtc

SEQ ID NO: 247 - DNA, 21 bp, synthetic construct
  source 1..21: mol_type = other DNA
ggcaaatgagaaacctaggcc

SEQ ID NO: 248 - DNA, 20 bp, synthetic construct
  source 1..20: mol_type = other DNA
aagtgggagctctgtagtca

SEQ ID NO: 249 - DNA, 23 bp, synthetic construct
  source 1..23: mol_type = other DNA
tcatcttgtgttgtttcccttca

SEQ ID NO: 250 - DNA, 22 bp, synthetic construct
  source 1..22: mol_type = other DNA
acttctgaagacctggctaaag

SEQ ID NO: 251 - DNA, 20 bp, synthetic construct
  source 1..20: mol_type = other DNA
gcccttttgcagtcagtttt

SEQ ID NO: 252 - DNA, 24 bp, synthetic construct
  source 1..24: mol_type = other DNA
gaaatgatccacttgtccataaat

SEQ ID NO: 253 - DNA, 27 bp, synthetic construct
  source 1..27: mol_type = other DNA
cacattgaacttctgattgatattatc

SEQ ID NO: 254 - DNA, 21 bp, synthetic construct
  source 1..21: mol_type = other DNA
cagagggagctgcttataaaa

SEQ ID NO: 255 - DNA, 23 bp, synthetic construct
  source 1..23: mol_type = other DNA
cagaaggtctctaagaaccaaac

SEQ ID NO: 256 - DNA, 27 bp, synthetic construct
  source 1..27: mol_type = other DNA
atgtatttaatacctatcttgttctca

SEQ ID NO: 257 - DNA, 22 bp, synthetic construct
  source 1..22: mol_type = other DNA
ctctcttagggccaatctcagt

SEQ ID NO: 258 - DNA, 20 bp, synthetic construct
  source 1..20: mol_type = other DNA
agagctttgaggccaggaaa

SEQ ID NO: 259 - DNA, 27 bp, synthetic construct
  source 1..27: mol_type = other DNA
agaaacaagaacttttacaatgatgct

SEQ ID NO: 260 - DNA, 19 bp, synthetic construct
  source 1..19: mol_type = other DNA
ctgacggctgacacttcac

SEQ ID NO: 261 - DNA, 26 bp, synthetic construct
  source 1..26: mol_type = other DNA
ttaatagtaataagatctgagcccca

SEQ ID NO: 262 - DNA, 26 bp, synthetic construct
  source 1..26: mol_type = other DNA
gtttctaaagacaggtcatcagagat

SEQ ID NO: 263 - DNA, 23 bp, synthetic construct
  source 1..23: mol_type = other DNA
catcgtagggataacagggtaat

SEQ ID NO: 264 - DNA, 23 bp, synthetic construct
  source 1..23: mol_type = other DNA
attaccctgttatccctacgatg

SEQ ID NO: 265 - DNA, 21 bp, synthetic construct
  source 1..21: mol_type = other DNA
catcgtaagcaaaccttagag

SEQ ID NO: 266 - DNA, 20 bp, synthetic construct
  source 1..20: mol_type = other DNA
gatcgaatcttctagccctt

SEQ ID NO: 267 - DNA, 23 bp, synthetic construct
  source 1..23: mol_type = other DNA
ggcaaataggaatggcaagaggg

SEQ ID NO: 268 - DNA, 21 bp, synthetic construct
  source 1..21: mol_type = other DNA
caaacagctgaccttgtgctt

SEQ ID NO: 269 - DNA, 22 bp, synthetic construct
  source 1..22: mol_type = other DNA
gagtggctccaacttccttgta

SEQ ID NO: 270 - DNA, 20 bp, synthetic construct
  source 1..20: mol_type = other DNA
gagatcgagaggtacggctg

SEQ ID NO: 271 - DNA, 20 bp, synthetic construct
  source 1..20: mol_type = other DNA
ctccaactgcccttctgtcc

SEQ ID NO: 272 - DNA, 19 bp, synthetic construct
  source 1..19: mol_type = other DNA
gtcatccctgagctgaacg

SEQ ID NO: 273 - DNA, 19 bp, synthetic construct
  source 1..19: mol_type = other DNA
gtcaaaggtggaggagtgg

SEQ ID NO: 274 - DNA, 23 bp, synthetic construct
  source 1..23: mol_type = other DNA
aattgctgatgaacccaataacc

SEQ ID NO: 275 - DNA, 23 bp, synthetic construct
  source 1..23: mol_type = other DNA
aattgctgatgaacccaaggtta

SEQ ID NO: 276 - DNA, 18 bp, synthetic construct
  source 1..18: mol_type = other DNA
gcaaaaccacgaaaccaa

SEQ ID NO: 277 - DNA, 17 bp, synthetic construct
  source 1..17: mol_type = other DNA
tgtggaatgtgtgcgag

SEQ ID NO: 278 - DNA, 21 bp, synthetic construct
  source 1..21: mol_type = other DNA
atcataatcagccataccaca

SEQ ID NO: 279 - DNA, 20 bp, synthetic construct
  source 1..20: mol_type = other DNA
catttagcaatcaacagcat

SEQ ID NO: 280 - DNA, 23 bp, synthetic construct
  source 1..23: mol_type = other DNA
tgtcaccaatcctgtccctagtg

SEQ ID NO: 281 - DNA, 23 bp, synthetic construct
  source 1..23: mol_type = other DNA
tgtcaccaatcctgtccccacta

SEQ ID NO: 282 - DNA, 20 bp, synthetic construct
  source 1..20: mol_type = other DNA
gtcacctctcactcctttca

SEQ ID NO: 283 - DNA, 19 bp, synthetic construct
  source 1..19: mol_type = other DNA
ccacaacgaggactacacc

SEQ ID NO: 284 - DNA, 23 bp, synthetic construct
  source 1..23: mol_type = other DNA
aagatcagccagactgagggtaa

SEQ ID NO: 285 - DNA, 23 bp, synthetic construct
  source 1..23: mol_type = other DNA
actctcttcgtggctttctgttt

SEQ ID NO: 286 - DNA, 23 bp, synthetic construct
  source 1..23: mol_type = other DNA
ttctccctcctcccctgcaagag

SEQ ID NO: 287 - DNA, 19 bp, synthetic construct
  source 1..19: mol_type = other DNA
cataattccaccacctctg

SEQ ID NO: 288 - DNA, 20 bp, synthetic construct
  source 1..20: mol_type = other DNA
gttacacagactagtgttca

SEQ ID NO: 289 - DNA, 23 bp, synthetic construct
  source 1..23: mol_type = other DNA
aagatcagccagactgagttacc

SEQ ID NO: 290 - DNA, 23 bp, synthetic construct
  source 1..23: mol_type = other DNA
ttctccctcctcccctgcctctt

SEQ ID NO: 291 - DNA, 19 bp, synthetic construct
  source 1..19: mol_type = other DNA
ctcggtcaccttcagcttg

SEQ ID NO: 292 - DNA, 19 bp, synthetic construct
  source 1..19: mol_type = other DNA
ctggacatcacctcccaca

SEQ ID NO: 293 - DNA, 23 bp, synthetic construct
  source 1..23: mol_type = other DNA
tactctacaactgaaagacagga

SEQ ID NO: 294 - DNA, 23 bp, synthetic construct
  source 1..23: mol_type = other DNA
agtgtcccatatccgcaatttta

SEQ ID NO: 295 - DNA, 23 bp, synthetic construct
  source 1..23: mol_type = other DNA
agaactgcttctaagatgccagc

SEQ ID NO: 296 - DNA, 23 bp, synthetic construct
  source 1..23: mol_type = other DNA
tgtcaccaaatttgtacctctaa

SEQ ID NO: 297 - DNA, 19 bp, synthetic construct
  source 1..19: mol_type = other DNA
ctctcttcaacggtgacac

SEQ ID NO: 298 - DNA, 22 bp, synthetic construct
  source 1..22: mol_type = other DNA
gatctgactttggactttattc

SEQ ID NO: 299 - DNA, 22 bp, synthetic construct
  source 1..22: mol_type = other DNA
actctacaactgaaagatcctg

SEQ ID NO: 300 - DNA, 23 bp, synthetic construct
  source 1..23: mol_type = other DNA
tgtcaccaaatttgtaccttaga